(19) United States
(12) Patent Application Publication
(10) Pub. No.: US 2024/0401001 A1
(43) Pub. Date: Dec. 5, 2024

(54) OPTIMIZED BIOSYNTHESIS PATHWAY FOR CANNABINOID BIOSYNTHESIS

(71) Applicant: Cellibre, Inc., San Diego, CA (US)

(72) Inventors: Spiros Kambourakis, San Diego, CA (US); Russell Scott Komor, San Diego, CA (US); Nicholas Donald Keul, San Diego, CA (US); Nicky Christopher Caiazza, Encinitas, CA (US); Jun Urano, Irvine, CA (US); Diep Minh Ngoc Nguyen, San Diego, CA (US)

(21) Appl. No.: 18/701,596

(22) PCT Filed: Oct. 17, 2022
(86) PCT No.: PCT/US2022/046924

§ 371 Date: Apr. 15, 2024

Related U.S. Application Data
(60) Provisional application No. 63/256,388, filed on Oct. 15, 2021.

(51) Int. Cl. C12N 9/02; C12N 9/10; C12N 9/12; C12N 9/20; C12N 15/52; C12P 7/42; C12P 17/06

(52) U.S. Cl. CPC C12N 9/0004; C12N 9/1085; C12N 9/1241; C12N 9/20; C12N 15/52; C12P 7/42; C12P 17/06; C12Y 121/03003; C12Y 205/01058; C12Y 207/07002; C12Y 301/01003; C07K 2319/00

(57) ABSTRACT

Provided herein are cells, enzymes, and methods for improved cannabinoid production.

### RELATED APPLICATIONS

This application claims the benefit of U.S. Provisional Application No. 63/256,388, filed Oct. 15, 2021, the entire teachings of which are incorporated herein by reference.

### BACKGROUND OF THE INVENTION

The Cannabaceae family of plants produces numerous different cannabinoids in variable relative quantities over a 7-10 week flowering period. Many of these cannabinoids have been and are currently being explored as therapeutics in chordates (e.g., mammals), and as a result, they are largely approved for medical and/or recreational use in the United States (Abrams DI Eur J Int Med 2018, 49, 7-11). Specifically, the most sought after (phyto)cannabinoids are: tetrahydrocannabinolic acid (THCA), cannabidiolic acid (CBDA), and cannabichromenic acid (CBCA). These phytocannabinoids and their associated chemical analogs are biosynthesized in various quantities from the same pre-cursor: cannabigerolic acid (CBGA). As a result, achieving high titers in the biosynthesis of THC(A), CBD(A) and CBC(A) either in the plant or in a recombinant host organism requires: (i) increasing the flux and availability of geranyl diphosphate (GPP) and olivetolic acid (OA) (ii) increasing the activity of CBGA synthase and (iii) increasing the activity and selectivity of THCA/CBDA/CBCA synthases. However, expression of the terminal plant synthases (THCAS, CBDAS, CBCAS) in recombinant organisms (yeast, E. coli etc.) has not been very successful, and only small amounts of product have been synthesized in whole cell biotransformations using these enzymes even after extensive engineering of the organism and the protein (Zirpel B et al., J. Biotechnol 2018, 40-47).

There remains a need for improving/modifying both the terminal synthases and the host organism, e.g., Yarrowia, for optimal expression and activity of enzymes by discovering novel sequences and/or improving enzyme activity.

### SUMMARY OF THE INVENTION

Some aspects of the present disclosure are directed to a cell expressing an exogenous terminal cannabinoid synthase and one or more chaperones. In some embodiments, the exogenous terminal cannabinoid synthase is selected from a berberine bridge enzyme (BBE)-like family enzyme selected from BBE1.6, BBE1.20, BBE1.21, BBE1.22, BBE2.1, BBE2.6, BBE2.7, BBE2.8, BBE2.16, BBE2.18, BBE2.19, BBE2.20, BBE2.21, BBE2.22, BBE3.1, BBE2.14, BBE25.1, BBE25.4, and BBE25.5 having an amino acid sequence corresponding to SEQ ID NO: 118, 19, 119, 120, 20, 25, 26, 27, 121, 122, 123, 124, 125, 126, 34, 33, 59, 62 or 63, respectively, or a functional fragment or derivative thereof having at least 70% sequence identity to one of these sequences.

In some embodiments, the exogenous terminal cannabinoid synthase has at least one amino acid modification (e.g., insertion, deletion, or substitution) as compared to wild type exogenous terminal cannabinoid synthase. In some embodiments, the exogenous terminal cannabinoid synthase has improved solubility, stability, turnover, selectivity, Km, and/or Kcat as compared to a wild type terminal cannabinoid synthase. In some embodiments, the exogenous terminal cannabinoid synthase is preferentially expressed in a location selected from the cytoplasm, ER, golgi, liposome, vacuole, plasma or outer cell membrane, peroxisome, oleosome, and the extracellular environment. In some embodiments, preferential expression involves a synthetic, heterologous or native signal peptide, retention sequence, leader peptide, or sorting sequence. In some embodiments, the exogenous terminal cannabinoid synthase is expressed with a signal peptide selected from SP3, SP4, SP7, SP8, or SP11.

In some embodiments, the exogenous terminal cannabinoid synthase is fused to a CBGA synthase, a secreted protein, a membrane protein, or a membrane localization sequence. In some embodiments, the cannabinoid synthase is fused to Lip2 (SEQ ID NO: 100), CWP1 (SEQ ID NO: 103), a 1,3-beta glucanosyltransferase (for example Uniprot Q6C8C9 or Q6CFU7), or a functional fragment of any of the above (e.g., having membrane localization or secretion activity and/or an N-terminal function fragment).

In some embodiments, the cell also over-expresses one or more chaperones selected from HAC1 (e.g., YALI0B12716p), HAC1s (e.g., SEQ ID NO: 105), FADS1 (e.g., YALI0D25564p), FADS1a (e.g., SEQ ID NO: 104), KAR2 (e.g., YALI0E13706p), FMN1 (e.g., YALI0B01826p), CNE1 (e.g., YALI0B13156p), ERO1 (e.g., YALI0D09603p), PDI1 (e.g., YALI0E03036p), IRE (e.g., YALI0A14839p), YAP1 (e.g., YALI0B03762p), HYR1 (e.g., YALI0E02310p), CsCHAP1 (e.g., XP_030509412.1 or SEGIDXX), CsCHAP2 (e.g., KAF4389684.1 or SEQ ID NO: 86), CsCHAP3 (e.g., KAF4346992.1 or SEQ ID NO: 87), CsDNAJ (e.g., XP_030510352.1), ClpB1 (e.g., XP_030489210.1), HSP90 (e.g., SRP155904_DN9237), or a functional fragment or derivative thereof. In some embodiments, the cell overexpresses HAC1 (YALI0B12716p) and/or CNE1 (YALI0B13156p) or a functional fragment or derivative thereof. In some embodiments, the chaperones are expressed with a signal protein selected from SP3, SP7, SP8, SP12 and with or without the HDEL motif. The latter is an ER retention sequence and can be added at the C-terminus sequence of the chaperones.

In some embodiments, the cell overexpresses Flavin adenine dinucleotide (FAD) chaperone or enzymes involved in the FAD biosynthesis. In some embodiments, the cell expresses an exogenous FAD synthetase or FMN synthetase or over-expresses a native FAD synthetase or FMN synthetase. In some embodiments, the exogenous FAD synthetase is Uniprot ID Q6C7T3 or FADS1 (YALI0D25564p) or FADS1a (SEQ ID NO: 104). In some embodiments, the FMN synthetase is Uniprot ID Q6CG11.

In some embodiments, the expression of one or more proteases selected from YALI0B05654p/AXP1, XPR2 (P09230), YALI0E33363p/AXP1-like, YALI0E28875p/XPR2-like, YALI0F27071p/PEP4, YALI0A06435p/PRB1A, YALI0B16500p/PRB1B, YALI0E34331p, YALI0E29403p, YALI0E28875p, YALI0E26851p, YALI0E21868p, YALI0E13552p, YALI0E13233p, YALI0E05423p, YALI0E04829p, YALI0E02024p, YALI0F26411p, YALI0F2¹⁶¹⁵p, YALI0F20592p, YALI0F19734p, YALI0F17974p, YALI0F16005p, YALI0F13585p, YALI0F11033p, YALI0F10769p, YALI0F07359p, YALI0F05940p, YALI0F01859p, YALI0F01540p, YALI0F00396p, YALI0F00176p, YALI0B20834p, YALI0B19228p, YALI0B17072p, YALI0B14641p, YALI0B13310p, YALI0B11594p, YALI0B10934p, YALI0B05522p, YALI0C10648p, YALI0C10494p, YALI0C09438p, YALI0C08283p, YALI0C05280p, YALI0C02519p, YALI0C00165p, YALI0D04807p, YALI0D07920p, YALI0D10967p, YALI0D13046p, YALI0D15642p, YALI0D16335p, YALI0D18832p, YALI0D19910p, YALI0D22957p, YALI0D23309p, YALI0C21604p, YALI0B04158p, YALI0B02574p, YALI0B01386p, YALI0A13277p, YALI0A10615p, YALI0E14388p2, YALI0B03718p, YALI0B16500p, YALI0D110835p, YALI0F09163p, YALI0E22374p, YALI0C00803g, YALI0D02024p, YALI0F11803g, YALI0C20273p, YALI0B14641g, YALI0F11803g, YALI0C20273g and YALI0C10923p is inhibited or inactivated in the cell. In some preferred embodiments, the expression of YALI0F09163p, and/or homologs and/or orthologs thereof, are inhibited or inactivated in the cell.

In some embodiments, the cell has been modified to inhibit or inactivate ROT2 glucosidase (YALI0B06600p). In some embodiments, the cell is capable of producing CBGA with either hexanoic acid or olivetolic acid supplementation or the cell is capable of producing CBGVA with butanoic or divarinic acid supplementation. In some embodiments, the cell is capable of producing CBDA/THCA/CBCA with OA supplementation and/or CBDVA/THCVA/CBCVA with DVA supplementation. In some embodiments, the cell is capable of producing CBDA/THCA/CBCA with hexanoic acid supplementation and/or CBDVA/THCVA/CBCVA with butyric acid supplementation.

In some embodiments, the cell has been engineered to enhance expression of the exogenous terminal cannabinoid synthase, wherein the engineering comprises one or more of: (1) improved import of the exogenous terminal cannabinoid synthase into a secretory pathway, (2) a modulated unfolded protein response, (3) a modulated disulfide bond formation activity, (4) a modulated FAD biosynthesis activity, (5) a modulated level of FAD covalent attachment to enzymes, (6) modulated or modified N-linked glycosylation, vesicle transport, protein degradation, lipid degradation, carbohydrate degradation, or heat shock proteins, (7) modulated reactive oxygen species pathway activity, and (8) modulated cellular protein sorting. As used herein, modulated means “increased or decreased.”

In some embodiments, the cell also expresses a prenyl transferase and produces CBGA or CBGVA by prenylating OA or DVA with GPP.

Some aspects of the present disclosure are directed to a method of producing CBDA, THCA, CBCA, CBDVA, THCVA, CBCVA, or analogs thereof, comprising contacting a cell disclosed herein with a carbon source and, optionally, hexanoic or butyric acid and suitable conditions to produce CBDA, THCA, CBCA, CBDVA, THCVA, CBCVA, or analogs thereof.

All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows biosynthesis pathways for CBGA and all major cannabinoids that are derived from it, including THCA, THCVA, CBCA, CBCVA, CBDA, and CBDVA.

FIG. 2 shows a list of cannabinoids that can be synthesized using CBGA synthase(s) described herein and in combination with a CBDA, CBCA, THCA, or other synthase.

### DETAILED DESCRIPTION OF THE INVENTION

### Some Definitions

“Identity” or “homology” refers to the extent to which the sequence of two or more nucleic acids or polypeptides is the same. In some embodiments, percent identity or homology between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity or homology, fractions are to be rounded to the nearest whole number. Percent identity or homology can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL ncbi.nlm.nih.gov for these programs. In a specific embodiment, percent identity or homology is calculated using BLAST2 with default parameters as provided by the NCBI.

The term “homolog” is intended to mean a nucleic acid sequence which possesses close sequence identity to the nucleic acid sequence of a recited gene and wherein both nucleic acid sequences are determined to be derived from the same ancestral gene, such as through speciation, either through phylogenetic analysis or through statistical analysis of the alignment between the sequences. When making the determination that two nucleic acids sequences are homologues through statistical analysis of the alignment between the sequences, tools which are widely known and available online, such as BLAST, may be utilized to make this determination. For purposes of this definition, alignments in BLAST given an expected value (E-value) of lower than 1×10-2, will be considered sufficient for determining that both nucleic acids derived from the same ancestral gene. The term “homolog” may also similarly be used to identify two amino acid sequences which possess close sequence homology and/or function and which are similarly determined to be encoded by and derived from the same ancestral gene. An “ortholog” is defined similarly as “homolog”, with the difference being the nucleic acid sequence which possesses close sequence identity to the nucleic acid sequence of a recited gene are both determined to be derived from the same ancestral gene through speciation.

The term “exogenous” is intended to mean that the referenced molecule or the referenced activity is introduced into the cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. When used in reference to a biosynthetic activity, the term refers to an activity that is introduced into the host. The source can be, for example, a homologous or heterologous encoding nucleic acid that expresses the referenced activity following introduction into the cell. Therefore, the term “endogenous” refers to a referenced molecule or activity that is present in the cell. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

The terms “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two-standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

### Cells Expressing Terminal Cannabinoid Synthases

Some aspects of the present disclosure are directed to a cell expressing an exogenous terminal cannabinoid synthase and one or more chaperones.

The cell is not limited and may be any suitable cell. In some embodiments, the cell is a bacteria, an algae, a yeast, or a plant cell. In some embodiments, the yeast is an oleaginous yeast (e.g., a Yarrowia lipolytica strain). In some embodiments, the bacteria is Escherichia coli.

Suitable cells may include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha (now known as Pichia angusta), Kluyveromyces sp., Kluyveromyces lactis, Kluyveromyces marxianus, Schizosaccharomyces pompe, Dekkera bruxellensis, Arxula adeninivorans, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, Yarrowia lipolytica and the like. In some embodiments, the cell is a protease-deficient strain of Saccharomyces cerevisiae. In some embodiments, the cell is a eukaryotic cell other than a plant cell. In some embodiments, the cell is a plant cell. In some embodiments, the cell is a plant cell, where the plant cell is one that does not normally produce a cannabinoid, a cannabinoid derivative or analogue, a cannabinoid precursor, or a cannabinoid precursor derivative or analogue. In some embodiments, the cell is Saccharomyces cerevisiae. In some embodiments, the cell disclosed herein is cultured in vitro.

In some embodiments, the cell is a prokaryotic cell. Suitable prokaryotic cells may include, but are not limited to, any of a variety of laboratory strains of Escherichia coli, Lactobacillus sp., Salmonella sp., Shigella sp., and the like. See, e.g., Carrier et al, (1992) J. Immunol. 148:1176-1181; U.S. Pat. No. 6,447,784; and Sizemore et al. (1995) Science 270:299-302. Examples of Salmonella strains which can be employed may include, but are not limited to, Salmonella typhi and S. typhimurium. Suitable Shigella strains may include, but are not limited to, Shigella flexneri, Shigella sonnei, and Shigella disenteriae. Typically, the laboratory strain is one that is non-pathogenic. Non-limiting examples of other suitable bacteria may include, but are not limited to, Bacillus subtilis, Pseudomonas putida, Pseudomonas aeruginosa, Pseudomonas mevalonii, Rhodobacter sphaeroides, Rhodobacter capsulatus, Rhodospirillum rubrum, Rhodococcus sp., and the like.

The terminal cannabinoid synthase is not limited and may be any suitable terminal cannabinoid synthase or functional fragment or derivative thereof. As used herein, a terminal cannabinoid synthase is a flavin adenine dinucleotide (FAD)-dependent berberine bridge enzyme that catalyzes the oxidative cyclization of the monoterpene moiety in CBGA.

In some embodiments, the exogenous terminal cannabinoid synthase is selected from a berberine bridge enzyme (BBE)-like family enzyme selected from BBE1.6, BBE1.20, BBE1.21, BBE1.22, BBE2.1, BBE2.6, BBE2.7, BBE2.8, BBE2.16, BBE2.18, BBE2.19, BBE2.20, BBE2.21, BBE2.22, BBE3.1, BBE2.14, BBE25.1, BBE25.4, and BBE25.5 having an amino acid sequence corresponding to SEQ ID NO: 118, 19, 119, 120, 20, 25-27, 121-126, 34, 33, 59, 62 or 63, respectively, or a functional fragment or derivative thereof having at least 70% sequence identity. In some embodiments, the exogenous terminal cannabinoid synthase or functional fragment or derivative thereof has an amino acid sequence with at least 75%, 80%, 85%, 90%, 95%, 99%, 99.5%, or 99.9% identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 106, 107, 108, 109, 110, 111, 112, 113, 114, 115, 116, 117, 118, 119, 120, 121, 122, 123, 124, 125, or 126. In some embodiments, the exogenous terminal cannabinoid synthase or functional fragment or derivative thereof has an amino acid sequence with at least 85%, 90%, 95%, 99%, or 99.9% identity to SEQ ID NO: 19, 20, 25, 26, 27, 32, 33, 59, 62, 63, 118, 119, 120, 121, 122, 123, 124, 125, or 126.

In some embodiments, the exogenous terminal cannabinoid synthase has at least one amino acid modifications (e.g., insertion, deletion, or substitution) as compared to wild type exogenous terminal cannabinoid synthase. Amino acid modifications may be amino acid substitutions, amino acid deletions and/or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g. charge, hydrophobicity and size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like.

In some embodiments, the exogenous terminal cannabinoid synthase or functional fragment or derivative thereof has improved solubility, stability, turnover, selectivity, Km, or Kcat as compared to a wild type terminal cannabinoid synthase. In some embodiments, the solubility of the exogenous terminal cannabinoid synthase or functional fragment or derivative thereof is at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or 10-fold greater than the solubility of a wild-type terminal cannabinoid synthase. In some embodiments, the stability of the exogenous terminal cannabinoid synthase or functional fragment or derivative thereof is at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or 10-fold greater than the stability of a wild-type terminal cannabinoid synthase. In some embodiments, the turnover of the exogenous terminal cannabinoid synthase or functional fragment or derivative thereof is at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or 10-fold less than the turnover of a wild-type terminal cannabinoid synthase. In some embodiments, the selectivity of the exogenous terminal cannabinoid synthase or functional fragment or derivative thereof is at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or 10-fold greater than the selectivity of a wild-type terminal cannabinoid synthase. In some embodiments, the Km of the exogenous terminal cannabinoid synthase or functional fragment or derivative thereof is at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or 10-fold less than the Km of a wild-type terminal cannabinoid synthase. In some embodiments, the K_{cat} of the exogenous terminal cannabinoid synthase or functional fragment or derivative thereof is at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, or 10-fold greater than the K_{cat} of a wild-type terminal cannabinoid synthase.

In some embodiments, the exogenous terminal cannabinoid synthase is preferentially expressed in a location selected from the cytoplasm, ER, Golgi, liposome, vacuole, plasma or outer cell membrane, peroxisome, oleosome, and the extracellular environment. In some embodiments, preferential expression involves a synthetic, heterologous or native signal peptide, retention sequence, leader peptide, or sorting sequence. In some embodiments, the exogenous terminal cannabinoid synthase is expressed with a signal peptide selected from SP3 (SEQ ID NO: 92), SP4 (SEQ ID NO: 92), SP7 (SEQ ID NO: 93), SP8 (SEQ ID NO: 95), or SP11 (SEQ ID NO: 96). For example, the exogenous terminal cannabinoid may be SP3-BBE1.6, SP3-BBE1.20, SP3-BBE1.21, SP3-BBE1.22, SP3-BBE2.1, SP3-BBE2.6, SP3-BBE2.7, SP3-BBE2.8, SP3-BBE2.16, SP3-BBE2.18, SP3-BBE2.19, SP3-BBE2.20, SP3-BBE2.21, SP3-BBE2.22, SP3-BBE3.1, SP3-BBE2.14, SP3-BBE25.1, SP3-BBE25.4, and SP3-BBE25.5, SP4-BBE1.6, SP4-BBE1.20, SP4-BBE1.21, SP4-BBE1.22, SP4-BBE2.1, SP4BBE2.6, SP4-BBE2.7, SP4-BBE2.8, SP4-BBE2.16, SP4-BBE2.18, SP4-BBE2.19, SP4-BBE2.20, SP4-BBE2.21, SP4-BBE2.22, SP4-BBE3.1, SP4-BBE2.14, SP4-BBE25.1, SP4-BBE25.4, and SP4-BBE25.5, SP7-BBE1.6, SP7-BBE1.20, SP7-BBE1.21, SP7-BBE1.22, SP7-BBE2.1, SP7-BBE2.6, SP7-BBE2.7, SP7-BBE2.8, SP7-BBE2.16, SP7-BBE2.18, SP7-BBE2.19, SP7-BBE2.20, SP7-BBE2.21, SP7-BBE2.22, SP7-BBE3.1, SP7-BBE2.14, SP7-BBE25.1, SP7-BBE25.4, and SP7-BBE25.5, SP8-BBE1.6, SP8-BBE1.20, SP8-BBE1.21, SP8-BBE1.22, SP8-BBE2.1, SP8-BBE2.6, SP8-BBE2.7, SP8-BBE2.8, SP8-BBE2.16, SP8-BBE2.18, SP8-BBE2.19, SP8-BBE2.20, SP8-BBE2.21, SP8-BBE2.22, SP8-BBE3.1, SP8-BBE2.14, SP8-BBE25.1, SP8-BBE25.4, and SP8-BBE25.5, SP11-BBE1.6, SP11-BBE1.20, SP11-BBE1.21, SP11-BBE1.22, SP11-BBE2.1, SP11-BBE2.6, SP11-BBE2.7, SP11-BBE2.8, SP11-BBE2.16, SP11-BBE2.18, SP11-BBE2.19, SP11-BBE2.20, SP11-BBE2.21, SP11-BBE2.22, SP11-BBE3.1, SP11-BBE2.14, SP11-BBE25.1, SP11-BBE25.4, and SP11-BBE25.5.

In some embodiments, the exogenous terminal cannabinoid synthase is fused to a CBGA synthase, a secreted protein, or a membrane localization sequence. In some embodiments, the cannabinoid synthase is fused to Lip2 (lipase 2, SEQ ID NO: 100), or CWP1 (cell wall protein 1, SEQ ID NO: 103) or a 1,3-beta glucanosyltransferase (Uniprot Q6C8C9 or Q6CFU7). In some embodiments, the exogenous terminal cannabinoid synthase is fused to a polyhistidine tag on the n-terminus or c-terminus of the enzyme. In some other embodiments, the exogenous terminal cannabinoid synthase is not fused to a polyhistidine tag. For example, while SEQ ID NO: 19, 20, 25-27, 121-126, 34, 33, 59, 62 and 63 corresponding to the amino acid sequences of BBE1.20, BBE2.1, BBE2.6, BBE2.7, BBE2.8, BBE2.16, BBE2.18, BBE2.19, BBE2.20, BBE2.21, BBE2.22, BBE3.1, BBE2.14, BBE25.1, BBE25.4, and BBE25.5, respectively contain c-terminal his-tags, these same enzymes without polyhis-tags are also envisioned. The polyhistidine tag may comprise two or more continuous histidine residues, two to eight continuous histidine residues, or two to six continuous histidine residues.

The term “chaperone” refers to a protein that assists in the folding of a protein or assembly of a complex (e.g., a protein-containing complex) but typically does not otherwise contribute to the final structure or function of the product. In some embodiments, the cell also over-expresses one or more chaperones selected from HAC1 (YALI0B12716p), HAC1s (SEQ ID NO: 105), FADS1 (YALI0D25564p), FADS1a (SEQ ID NO: 104), KAR2 (YALI0E13706p), FMN1 (YALI0B01826p), CNE1 (YALI0B13156p), ERO1 (YALI0D09603p), PDI1 (YALI0E03036p), IRE (YALI0A14839p), YAP1 (YALI0B03762p), HYR1 (YALI0E02310p), CsCHAP1 (XP_030509412.1 or SEQ ID NO: 85), CsCHAP2 (KAF4389684.1 or SEQ ID NO: 86), CsCHAP3 (KAF4346992.1 or SEQ ID NO: 87), CsDNAJ (XP_030510352.1), ClpB1 (XP_030489210.1), HSP90 (SRP155904_DN9237), or a functional fragment or derivative thereof. In some embodiments, the cell over-expresses HAC1 (YALI0B12716p) and/or CNE1 (YALI0B¹³¹⁵⁶p) or a functional fragment or derivative thereof. In some embodiments, the chaperones are expressed with a signal protein selected from SP3 (SEQ ID NO: 92), SP7 (SEQ ID NO: 94), SP8 (SEQ ID NO: 95), or SP-KAR2 (SEQ ID NO: 97) and with or without the ER retention HDEL motif.

In some embodiments, the cell expresses or overexpresses one or more chaperones or homologs thereof involved in covalent attachment of FAD to terminal cannabinoid synthases. In some embodiments, the chaperones or homologs thereof are selected from CsCHAP1, CsCHAP2 CsCHAP3, CsDNAJ1, CsDNAJ2, CsCLB1.1, CsCLB1.2, CsCLB1.3, CsHSP70_1, CsHSP70_2, CsHSP70_3, CsHSP70_4, CsHSP70_5, and FADS. In some embodiments, the chaperones or homologs thereof are selected from the chaperones provided in TABLE 1 herein.

In some embodiments, the cell expresses or overexpresses one or more enzymes involved in FAD biosynthesis. In some embodiments, the cell expresses an exogenous FAD synthetase or FMN synthetase or over-expresses a native FAD synthetase or FMN synthetase. In some embodiments, the exogenous FAD synthetase is Uniprot ID Q6C7T3 or FADS1 (YALI0D25564p) or FADS1a (SEQ ID NO: 104). In some embodiments, the FMN synthetase is Uniprot ID Q6CG11. In some embodiments, the one or more enzymes involved in FAD biosynthesis is a FAD synthetase or FMN biosynthesis enzyme provided in TABLE 1 herein.

In some embodiments, the expression in the cell is inhibited or inactivated of one or more proteases selected from YALI0B05654p/AXP1, XPR2 (P09230), YALI0E33363p/AXP1-like, YALI0E28875p/XPR2-like, YALI0F27071p/PEP4, YALI0A06435p/PRB1A, YALI0B16500p/PRB1B, YALI0E34331p, YALI0E29403p, YALI0E28875p, YALI0E26851p, YALI0E21868p, YALI0E13552p, YALI0E13233p, YALI0E05423p, YALI0E04829p, YALI0E02024p, YALI0F26411p, YALI0F2¹⁶¹⁵p, YALI0F20592p, YALI0F19734p, YALI0F17974p, YALI0F16005p, YALI0F13585p, YALI0F11033p, YALI0F10769p, YALI0F07359p, YALI0F05940p, YALI0F01859p, YALI0F01540p, YALI0F00396p, YALI0F00176p, YALI0B20834p, YALI0B19228p, YALI0B17072p, YALI0B14641p, YALI0B13310p, YALI0B11594p, YALI0B10934p, YALI0B05522p, YALI0C10648p, YALI0C10494p, YALI0C09438p, YALI0C08283p, YALI0C05280p, YALI0C02519p, YALI0C00165p, YALI0D04807p, YALI0D07920p, YALI0D10967p, YALI0D13046p, YALI0D15642p, YALI0D16335p, YALI0D18832p, YALI0D19910p, YALI0D22957p, YALI0D23309p, YALI0C21604p, YALI0B04158p, YALI0B02574p, YALI0B01386p, YALI0A13277p, YALI0A10615p, YALI0E14388p2, YALI0B03718p, YALI0B16500p, YALI0D10835p, YALI0F09163p, YALI0E22374p, YALI0C00803g, YALI0D02024p, YALI0F11803g, YALI0C20273p, YALI0B14641g, YALI0F11803g, YALI0C20273g, YALI0C10923p, and homologs and orthologs thereof. In some embodiments, the expression of YALI0F09163p, and/or homologs and/or orthologs thereof are inhibited or inactivated in the cell. some embodiments, the expression of the one or more proteases is inhibited by at least about 10%, 15%, 20%, 25%, 30%, 35%, 40%, 45%, 50%, 55%, 60%, 65%, 70%, 75%, 80%, 85%, 90%, 95%, 99%, or more as compared to a reference level.

In some embodiments, the cell has been modified to inactivate or reduce activity/expression of ROT2 glucosidase (YALI0B06600p). In some embodiments, the expression or activity of the ROT2 glucosidase is reduced by at least about 10%, 15%, 20%, 25%, 30%, 35%, 40%, 45%, 50%, 55%, 60%, 65%, 70%, 75%, 80%, 85%, 90%, 95%, 99%, or more as compared to a reference level.

In some embodiments, the cell is capable of producing CBGA with either hexanoic acid or olivetolic acid supplementation. In some embodiments, the cell is capable of producing CBGVA with butanoic or divarinic acid (DVA) supplementation. In some embodiments, the cell is capable of producing CBDA, THCA, and/or CBCA with OA supplementation. In some embodiments, the cell is capable of producing CBDVA, THCVA, and/or CBCVA with DVA supplementation. In some embodiments, the cell is capable of producing CBDA, THCA, and/or CBCA with hexanoic acid supplementation. In some embodiments, the cell is capable of producing CBDVA, THCVA, and/or CBCVA with butyric acid supplementation.

In some embodiments, the cell has been engineered to enhance expression of the exogenous terminal cannabinoid synthase, wherein the engineering comprises one or more of: (1) improved import of the exogenous terminal cannabinoid synthase into a secretory pathway, (2) a modulated unfolded protein response, (3) a modulated disulfide bond formation activity, (4) a modulated FAD biosynthesis activity, (5) a modulated level of FAD covalent attachment to enzymes, (6) modulated or modified N-linked glycosylation, vesicle transport, protein degradation, lipid degradation, carbohydrate degradation, or heat shock proteins, (7) modulated reactive oxygen species pathway activity, and (8) modulated cellular protein sorting.

In some embodiments, the cell also expresses a prenyl transferase and is capable of producing CBGA or CBGVA by prenylating OA or DVA with GPP. In some embodiments, the prenyl transferase is a prenyl transferase provided in WO 2021/178976 published Sep. 10, 2021, herein incorporated by reference.

Some aspects of the present disclosure are directed to production of one or more cannabinoids with a cell disclosed herein. Cannabinoids, cannabinoid derivatives and cannabinoid analogues as recited herein are not limited. In some embodiments, cannabinoids may include, but are not limited to, cannabichromene (CBC) type (e.g. cannabichromenic acid), cannabigerol (CBG) type (e.g. cannabigerolic acid), cannabidiol (CBD) type (e.g. cannabidiolic acid), Δ⁹-trans-tetrahydrocannabinol (Δ⁹-THC) type (e.g. Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol (CBT) type, cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄(CBD-C₄), cannabidivarinic acid (CBDVA), cannabidivarin (CBDV), cannabidiorcol (CBD-C₁), Δ⁹-tetrahydrocannabinolic acid A (THCA-A), Δ⁹-tetrahydrocannabinolic acid B (THCA-B), Δ⁹-tetrahydrocannabinol (THC), Δ⁹-tetrahydrocannabinolic acid-C₄(THCA-C₄), Δ⁹-tetrahydrocannabinol-C₄(THC-C₄), Δ⁹-tetrahydrocannabivarinic acid (THCVA), Δ⁹-tetrahydrocannabivarin (THCV), Δ⁹-tetrahydrocannabiorcolic acid (THCA-C₁), Δ⁹-tetrahydrocannabiorcol (THC-C₁), Δ⁷-cis-iso-tetrahydrocannabivarin, Δ⁸-tetrahydrocannabinolic acid (Δ⁸-THCA), Δ⁸-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C₄, (CBN-C₄), cannabivarin (CBV), cannabinol-C₂ (CNB-C₂), cannabiorcol (CBN-C₁), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC).

An expression vector or vectors can be constructed to include exogenous nucleotide sequences coding for the recombinant polypeptides described herein operably linked to expression control sequences functional in the cell. Expression vectors applicable include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

In some embodiments, the cell described herein comprises one or more additional metabolic pathway transgene(s). In some embodiments, the cell comprises an olivetolic acid pathway. In some embodiments, the olivetolic acid pathway comprises a polyketide cyclase. In some embodiments, an exogenous nucleotide codes for the polyketide cyclase. In some embodiments, the olivetolic acid pathway comprises polyketide synthase/olivetol synthase (condensation of hexanoyl coenzyme A (CoA) and 3× malonyl CoAs). In some embodiments, the cell comprises a geranyl pyrophosphate (GPP) pathway. In some embodiments, the GPP pathway comprises geranyl pyrophosphate synthase. In some embodiments, an exogenous nucleotide codes for the geranyl pyrophosphate synthase. In some embodiments, the cell comprises a farnesyl pyrophosphate (FPP) pathway. In some embodiments, the FPP pathway comprises a farnesyl pyrophosphate synthase. In some embodiments, the farnesyl pyrophosphate synthase is a mutant form. In some embodiments, the mutant farnesyl pyrophosphate synthase is described in (Jian G-Z, et al Metabolic Engineering, 2017, 41, 57, incorporated herein). In some embodiments, an exogenous nucleotide codes for the farnesyl pyrophosphate synthase. In some embodiments, the cell comprises a divarinic acid (DVA) pathway. In some embodiments, the DVA pathway comprises divarinic acid synthase. In some embodiments, an exogenous nucleotide codes for the divarinic acid synthase. In some embodiments, the cell comprises a mevalonate pathway. In some embodiments, the cell expresses HMG-CoA reductase. In some embodiments, an endogenous mevalonate pathway of the cell has been manipulated to reduce or increase production of mevalonate, isopentyl pyrophosphate (IPP) or dimethylallyl pyrophosphate (DMAP), geranyl pyrophosphate (GPP) or farnesyl pyrophosphate (FPP). In some embodiments, the cell comprises a polyketide cyclase that produces OA, DVA, and/or derivatives thereof. In some embodiments, the cell comprises a polyketide synthase that produces a tetraketide substrate of the polyketide cyclase. In some embodiments, the cell comprises a polytetide synthase that can directly form OA and derivatives from acetyl-CoA or hexanoyl-CoA and malonyl-CoA. In some embodiments, the cell has a modified native GPP/FPP synthase that preferentially produces GPP as compared to the native GPP/FPP synthase. Examples of modified native GPP/FPP synthases that preferentially produce GPP as compared to the native GPP/FPP synthase, and cells which express them, are described in commonly owned U.S. Provisional Application 63/256,398, which is hereby incorporated by reference in its entirety.

Some aspects of the present disclosure are directed to a method of producing CBDA, THCA, CBCA, CBDVA, THCVA, CBCVA, or analogs thereof, comprising contacting a cell disclosed herein with a carbon source and, optionally, hexanoic or butyric acid, under suitable conditions to produce CBDA, THCA, CBCA, CBDVA, THCVA, CBCVA, or analogs thereof.

Depending on the cell, the appropriate culture medium may be used. For example, descriptions of various culture media may be found in “Manual of Methods for General Bacteriology” of the American Society for Bacteriology (Washington D.C., USA, 1981). As used here, “medium” as it relates to the growth source refers to the starting medium be it in a solid or liquid form. “Cultured medium”, on the other hand and as used here refers to medium (e.g. liquid medium) containing microbes that have been fermentatively grown and can include other cellular biomass. The medium generally includes one or more carbon sources, nitrogen sources, inorganic salts, vitamins and/or trace elements.

Exemplary carbon sources include sugar carbons such as sucrose, glucose, galactose, fructose, mannose, isomaltose, xylose, panose, maltose, arabinose, cellobiose and 3-, 4-, or 5-oligomers thereof. Other carbon sources include alcohol carbon sources such as methanol, ethanol, glycerol. Other carbon sources include acid and esters such as acetate, formate, fatty acids having four to twenty-two carbon atoms or fatty acid esters thereof. Other carbon sources can include renewal feedstocks and biomass. Exemplary renewal feedstocks include cellulosic biomass, hemicellulosic biomass and lignin feedstocks. Mixed carbon sources can also be used, such as a fatty acid and a sugar as described herein.

The culture conditions can include, for example, liquid culture procedures as well as fermentation and other large-scale culture procedures. Useful yields of the products can be obtained under aerobic culture conditions. An exemplary growth condition for achieving one or more cannabinoid products includes aerobic culture or fermentation conditions. In certain embodiments, the microbial organism can be sustained, cultured or fermented under aerobic conditions.

Substantially aerobic conditions include, for example, a culture, batch fermentation or continuous fermentation such that the dissolved oxygen concentration in the medium remains between 5% and 100% of saturation. The percent of dissolved oxygen can be maintained by, for example, sparging air, pure oxygen or a mixture of air and oxygen.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art.

In some embodiments, the methods further comprise a step of purifying or isolating the cannabinoids, derivatives or analogues thereof from the culture. Methods of isolation are not limited and may be any suitable method known in the art. Purification methods include, for example, extraction procedures as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration or centrifugal partition chromatography (CPC).

In some embodiments, the cells are grown in stirred tank fermenters with feed supplementation (sugars with or without organic acids) where the dissolved oxygen, temperature, and pH are controlled according to the optimal growth and production process. In some embodiments, aqueous non-miscible organic solvents are supplemented to dissolve added organic acids or extract the cannabinoid products as they are being synthesized. In some embodiments, these solvents may include, but are not limited to, isopropyl myristate (IPM), diisobutyl adipate, decane, dodecane, hexadecane or anther organic solvent with log P>5. The later number (log P) is defined as the log of a compound's partition between water and octanol and is a standard parameter of a compound's hydrophobicity (the larger the log P the less soluble in water). Depending on the fermentation process, the products can be isolated and purified using different methods.

If no organic cosolvent is used and the targeted cannabinoid(s) is being secreted to the culture supernatant, different methods can be applied. In one embodiment, an aqueous miscible organic solvent (ethanol, acetonitrile, etc.) is added to dissolve the products. In some embodiments, a simple filtration, ultrafiltration or centrifugation can remove the cells and the aqueous media evaporated to dryness or to a small volume from which the cannabinoid product will precipitate or crystalize. Alternatively, the cell supernatant can be extracted with an aqueous immiscible organic solvent (ethyl acetate, heptane, butyl-acetate, propyl-acetate, methyl isobutyl ketone etc.) to extract the cannabinoids. Evaporation of the organic solvent and a possible recrystallization will produce pure cannabinoid. If the cannabinoid products are not secreted to the media and are trapped inside the cell, different methods for their extraction and purification can be utilized. In some embodiments, cells are disrupted using mechanical methods or by suspension in appropriate lysis buffers from which the cannabinoids can be extracted with an organic aqueous immiscible solvent (ethyl acetate, hexane, decane, methylene chloride, etc.). In other embodiments, cells may be suspended in an organic solvent (ethanol, methanol, methylene chloride, etc.) that extracts the cannabinoids from the cells.

In some embodiments, an organic solvent is required during growth that is separated at the end of the fermentation. Back extraction with basic aqueous solvent or a different organic solvent with low boiling point and high polarity (ethanol, acetonitrile, etc.) will remove the cannabinoids. Isolation can then involve a simple pH shift if water is used, or an evaporation if organic solvents are used. In both cases, a recrystallization step may be required at the end to improve purity of the product.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, or cell, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Introduction

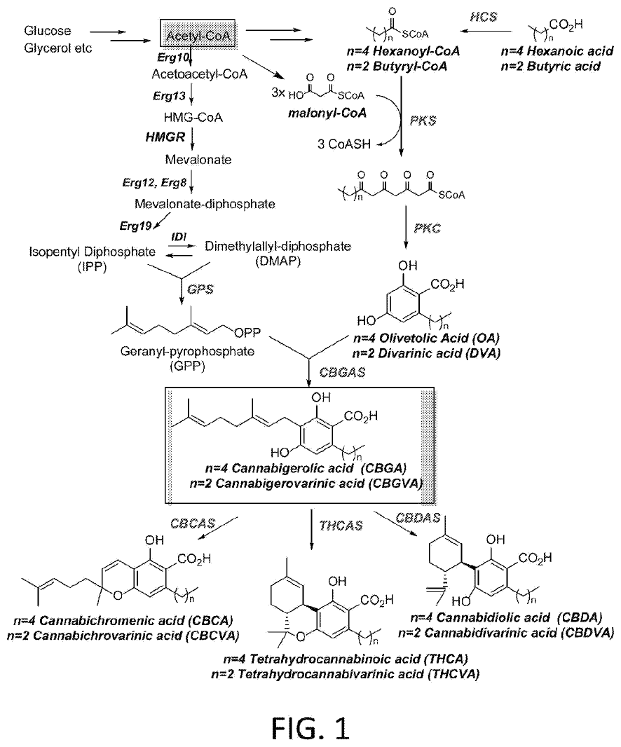

The terminal synthases (CBDAS, THCAS, and CBCAS) convert CBGA and its analogs into the final products CBDA, THCA, and CBCA and analogs (FIG. 1). These synthases from Cannabis plants are highly homologous (>=70% seq. identity), contain bi-covalently bound FAD (6-S-cysteinyl, 8alpha-N1-histidyl FAD linkage), and belong in the berberine bridge enzyme (BBE)-like family as determined when the crystal structure of THCAS was solved (Shoyama Y, et al J. Mol Biol 2012, 423, 96-105; [PDB: 3VTE]). In addition to Cannabis, BBE-like enzymes from other sources, including other eukaryotes or prokaryotes are described herein.

So far recombinant expression of these enzymes in yeast (Saccharomyces or Pichia) has been problematic and required significant modification/upregulation of certain chaperones of the ER/secretion and FAD biosynthesis pathways (Zirpel B, et al J. Biotechnol 2018, 40-47). Extensive mutagenesis of CBDA and THCA synthase genes showed only marginal improvements of activity and corresponding titers of cannabinoids when expressed in yeast (WO2020236789).

Available information therefore suggests that expression of the terminal synthases is problematic in microbes. Thus, Applicants note that expression may require accessory proteins and conditions present in the Cannabis plant such as (1) folding and stabilizing chaperones and (2) adequate/increased amounts of intracellular FAD (Table 1).

Of known FAD containing enzymes, roughly 10% have this cofactor covalently attached to a single amino acid of the protein. Far fewer enzymes have two covalent attachments, as is the case of the synthases from Cannabis (Starbird, C A et al eLS John Wiley & Sons, Ltd 2015 DOI: 10.1002/9780470015902.a0026073). Due to this particularity, Cannabis terminal synthases may require specific chaperones present in the Cannabis plant. These chaperones may play a role in stabilizing the protein's conformation, allowing FAD attachment during or after protein folding. They may also play an active role in catalyzing the covalent FAD attachment. Chaperones that are involved in the covalent attachment of FAD have been identified in the flavination of succinate dehydrogenase. These are small enzymes (˜10 KDa) that are present in all species (bacteria, yeast, mammals) and have been shown to promote the covalent attachment of the 8a methyl of FAD into an active site Histidine (McNeil M B, Fineran P C Biochemistry 2013, 52(43), 7628-7640).

To identify similar sequences or related chaperones that may help in the FAD attachment to the terminal synthases in Cannabis, the proteome and transcriptome of different Cannabis cultivars were searched with a focus on proteins expressed in trichomes. This approach identified several chaperones that will be expressed together with the terminal synthases.

These enzymes include homologs to chaperones that aid FAD attachment to succinate dehydrogenase (CsCHAP1, CsCHAP2 CsCHAP3). Other relevant chaperones from Cannabis include CsDNAJ1, CsDNAJ2, CsCLB1.1, CsCLB1.2, CsCLB1.3, CsHSP70_1, CsHSP70_2, CsHSP70_3, CsHSP70_4 and CsHSP70_5 and FADS. The equivalent enzymes from Yarrowia will also be over-expressed (Table 1).

Functional expression of the terminal synthases may require, or be improved, through engineering the secretion pathway of the host organism. There are numerous proteins and enzymes in the secretion pathway(s) that can be targets for enzyme engineering, altering gene expression (up regulation or down regulation), functional inactivation, and/or heterologous gene expression. These target enzymes may be involved in, but not limited to, import of CBDAS into the secretory pathway, the unfolded protein response, disulfide bond formation, FAD biosynthesis and covalent attachment to enzymes, N-linked glycosylation/modification, vesicle transport, protein degradation, lipid degradation, carbohydrate degradation, protein folding chaperones, heat shock proteins, the reactive oxygen species pathway (ROS signaling upregulation/downregulation), cellular protein sorting, etc. A list of these enzymes is shown in Table 1.

Additionally, the terminal synthases may be targeted to different locations in the host cell including the cytoplasm, ER, golgi, liposome, vacuole, plasmid membrane, peroxisome, or the extracellular environment. Targeting can be achieved by adding a wide range of signal peptides, retention sequences, or sorting sequences that may be native to the host, heterologous to the host, and/or synthetic and/or engineered sequences.

TABLE 1
Enzymes that will be targeted for expression

| Enzyme (Uniprot) | Organism | Modification | Function |
|---|---|---|---|
| YALI0B12716p/HAC1 | Yarrowia | Over- | Unfolded |
| | | expression | protein |
| | | | response |
| HAC1s/SEQ ID NO: | Yarrowia | Over- | Unfolded |
| 105 | | expression | protein |
| | | | response |
| YALI0D25564p/FADS1 | Yarrowia | Over- | FAD |
| | | expression | synthetase |
| FADS1a/SEQ ID NO: | Yarrowia | Over- | FAD |
| 104 | | expression | synthetase |
| YALI0E13706p/KAR2 | Yarrowia | Over- | ER |
| | | expression | Chaperone |
| YALI0B01826p/FMN1 | Yarrowia | Over- | FMN |
| | | expression | biosynthesis |
| YALI0B13156p/CNE1 | Yarrowia | Over- | Unfolded |
| | | expression | protein |
| | | | response |
| YALI0D09603p/ERO1 | Yarrowia | Over- | ER |
| | | expression | Chaperone |
| YALI0E03036p/PDI1 | Yarrowia | Over- | Disulfide |
| | | expression | bond |
| | | | isomerase |
| YALI0A14839p/IRE | Yarrowia | Over- | Unfolded |
| | | expression | protein |
| | | | response |
| YALI0B03762p/YAP1 | Yarrowia | Over- | Oxidative |
| | | expression | stress |
| | | | response |
| YALI0E02310p/HYR1 | Yarrowia | Over- | Glutathione |
| | | expression | peroxidase |
| CsCHAP1 | Cannabis | Over- | Chaperone |
| (XP_030509412.1) | | expression | |
| CsCHAP2 | Cannabis | Over- | Chaperone |
| (KAF4389684.1) | | expression | |
| CsCHAP3 | Cannabis | Over- | Chaperone |
| | | expression | |
| FAD synthase | Cannabis | Over- | FAD |
| (XP_030493922.1) | | expression | synthase |
| DNAJ | Cannabis | Over- | Chaperone |
| (XP_030510352.1) | | expression | |
| ClpB1 | Cannabis | Over- | Chaperone |
| (XP_030489210.1) | | expression | |
| HSP90 | Cannabis | Over- | Chaperone |
| (SRP155904_DN9237) | | expression | |

*CsCHAP1 & CsCHAP2 will be expressed using 4 different signal peptides (SP3, SP7, SP8, SP-KAR2) and ± HDEL motif.

TABLE 2
Proteases that will be inactivated

| Secreted | Non-secreted |
|---|---|
| YALI0D10967p | YALI0E34331p YALI0E29403p YALI0E28875p |
| (Q6C9I4), | YALI0E26851p YALI0E21868p YALI0E13552p |
| YALI0B16500p | YALI0E13233p YALI0E05423p YALI0E04829p |
| (Q6CED6), | YALI0F26411p YALI0F21615p YALI0F20592p |
| YALI0D10835p | YALI0F19734p YALI0F17974p YALI0F16005p |
| (Q6C9J0), | YALI0F13585p YALI0F11033p YALI0F10769p |
| YALI0E33363p | YALI0F07359p YALI0F05940p YALI0F01859p |
| (Q6C3N7), | YALI0F01540p YALI0F00396p YALI0F00176p |
| YALI0F09163p | YALI0B20834p YALI0B19228p YALI0B17072p |
| (Q6C2B8), | YALI0B14641p YALI0B13310p YALI0B11594p |
| YALI0E22374p | YALI0B10934p YALI0B05522p YALI0C10648p |
| (Q6C4Z7), | YALI0C10494p YALI0C09438p YALI0C08283p |
| YALI0C00803g | YALI0C05280p YALI0C02519p YALI0C00165p |
| (Q6CDG1), | YALI0D04807p YALI0D07920p YALI0D13046p |
| YALI0D02024p | YALI0D15642p YALI0D16335p YALI0D18832p |
| (Q6CAK4), | YALI0D19910p YALI0D22957p YALI0D23309p |
| YALI0F11803g | YALI0C21604p YALI0B04158p YALI0B02574p |
| (Q6C209), | YALI0B01386p YALI0A13277p YALI0A10615p |
| YALI0C20273p | YALI0E14388p2 YALI0B03718p |
| (Q6CBB7), | |
| YALI0B14641g | |
| (Q6CEL9), | |
| YALI0F11803g | |
| (Q6C209), | |
| YALI0C20273g | |
| (Q6CBB7), | |
| YALI0C10923p | |
| (Q6CCB2) | |

In addition to upregulating or downregulating native chaperones, or expressing chaperones from yeast, the amino acid sequences of CBDA, THCA and CBCA synthases will also be modified. There are numerous isozymes of each of these enzymes in sequenced Cannabis plants and various of these enzymes will be tested for expression in the modified strains. In addition, enzymes in the BBE family with potential CBGA cyclization activity from plants, fungi and microbes were identified using different bioinformatics and AI techniques. The list of synthases that will be tested include, but are not limited to, the enzymes in the BBE family described herein (BBE1.1-BBE58; SEQ ID NOS: 1-84 and SEQ ID NOS: 106-126).

Mutagenesis of these enzymes at selected positions (at the protein surface, adding/removing glycosylation sites, add disulfide bonds, active site, etc.) will also be performed to improve both the physical (solubility, stability) and catalytic (turnover, Km, Kcat) properties of these proteins.

Different methods for increasing the activity or improve the targeting of the synthases were adopted. In one approach, the enzymes were fused with different lead sequences that target the protein in specific compartments of the cell (peroxisome, oleosome, etc), the cell membranes, or the extracellular space or media. Secondly the synthases were fused with different proteins such as CBGA synthase to increase the activity by substrate (CBGA) channeling, or were fused with proteins that can target the enzymes in different compartments such as the outer cell membranes or secrete them to the supernatant.

### Example 1: Expression of THCAS in YL and Synthesis of THCA from Hexanoic Acid or OA Feed

Plasmids pCL-SE-0696 and pCL-SE-0703 through -0706 express BBE2.1 with different signal peptides. These plasmids were linearized with DraI then transformed into strain SB-691 and multiple clones were screened for THCA production. SB-691 has been engineered such that it can produce CBGA with either hexanoic acid or olivetolic acid supplementation.

### THCA Formation from a Hexanoic Acid Feed

Patched colonies were used to inoculate 0.5 mL YPD (peptone 20 g/L, yeast extract 10 g/L, glucose 20 g/L) media in 96dw blocks which were grown at 30° C. with 1000 rpm shaking. After 48 h, 5 μL from each preculture was used to inoculate 0.5 mL YPD media with 100 mM MES (pH 5.5), and 2.5 mM hexanoic acid in 96 deep well plates (2 mL), which were grown at 30° C. with 1000 rpm shaking. Cultures were supplemented with an additional 2% glucose and 5 mM hexanoic acid three times by adding 20 μL of 50% glucose with 12 mM hexanoic acid at 24, 48, and 72 h. After an additional 24 h (96 h total), cultures were quenched with 0.5 mL ethanol with 0.1% formic acid and 0.1 mg/mL pentyl-benzoic acid and submitted for LC analysis. Table 3 lists the highest concentration of THCA produced among the different transformants screened of each strain.

TABLE 3
THCA formation from hexanoic acid feed. Products
in μM accumulated in the in vivo assay.

| | Upstream pathway | BBE | |
|---|---|---|---|
| Strain | enzymes introduced (1) | Expressed | THCA |
| SB-691 | HCS2, PKS1, PKC1.1, | | 0 |
| | HMGR, ERG20ww, | | |
| | ERG20ww-MPT4 | | |
| SB-0824 | HCS2, PKS1, PKC1.1, | SP3.BBE2.1 | 192 |
| | HMGR, ERG20ww, | | |
| | ERG20ww-MPT4 | | |
| SB-0825 | HCS2, PKS1, PKC1.1, | SP4.BBE2.1 | 224 |
| | HMGR, ERG20ww, | | |
| | ERG20ww-MPT4 | | |
| SB-0826 | HCS2, PKS1, PKC1.1, | SP7.BBE2.1 | 180 |
| | HMGR, ERG20ww, | | |
| | ERG20ww-MPT4 | | |
| SB-0827 | HCS2, PKS1, PKC1.1, | SP8.BBE2.1 | 199 |
| | HMGR, ERG20ww, | | |
| | ERG20ww-MPT4 | | |
| SB-1045 | HCS2, PKS1, PKC1.1, | SP11.BBE2.1 | 97 |
| | HMGR, ERG20ww, | | |
| | ERG20ww-MPT4 | | |

(1) Detailed description of these enzymes and constructs are described in commonly owned U.S. Provisional patent Application No. 63/188,645 filed May 14, 2021 or commonly owned U.S. Provisional patent Application No. 63/188,648 filed May 14, 2021, both incorporated herein by reference in their entireties.

THCA and THCVA Formation from an OA and DVA Feed, Respectively.

Patched colonies of strains harboring the entire cannabinoid biosynthetic pathway including the terminal synthases described herein that produce the final cannabinoid(s) (e.g., THCA and THCVA) were used to inoculate cultures containing 0.5 mL YPD media (2% glucose) a sterile 96 deep-well (DW) plate. The inoculated cultures in the 96 DW plate were then sealed with a breathable, sterile seal and placed into a high-speed shaker set to 30° C. set to a shake speed of 1000 rpm. The pre-cultures were grown for 48 hrs under the same conditions and 20 μL from each well was used as an inoculum into another plate containing 0.5 mL of YPD (2%)+100 mM MES pH=5.5+250 mg/L Thiamine+0.1 M Betaine glycine+5 μL of protease inhibitor cocktail and either olivetolic acid (OA) or divarinic acid (DVA) at a final concentration 3 mM. The plate was then placed in the high-speed shaker, which was set at 30° C. and a shake speed of 1000 rpm. The reactions with OA were terminated at t=48 hrs via quenching with the following quench buffer: 0.5 mL ethanol with 0.1% formic acid and 0.1 mg/mL pentyl-benzoic acid and then subsequently submitted for LC/MS analyses. The reactions with DVA were quenched at t=96 hrs. Clonal variation from random genomic integration was assessed for each of the SPX.BBEX.X in the relevant SB's. The following results represent the top clones from an OA & DVA feed. Table 4 lists the highest concentration of either THCA or THCVA produced among the different transformants screened of each strain with either an OA or a DVA feed, respectively.

TABLE 4
THCA formation from an OA and/or DVA feed.
Products in μM accumulated in the in vivo assay.
OA and DVA Feed Experiments ^{a}

| | | [THCA], | [THCVA], |
|---|---|---|---|
| | Enzyme | μM | μM |
| Strain | Expressed | Produced ^{b} | Produced ^{c} |
| SB-0824 | SP3.BBE2.1 | 391.7 ± 3.8 | 193.6 |
| SB-0825 | SP4.BBE2.1 | 249.6 ± 2.7 | |
| SB-0826 | SP7.BBE2.1 | 186.8 ± 10.8 | |
| SB-0827 | SP8.BBE2.1 | 159.9 ± 11.7 | |

^{a} Top randomly integrated clones with the enzyme(s) expressed are represented.
^{b} The reactions with OA were quenched at t = 60 hrs
^{c} The reaction with DVA was quenched at t = 96 hrs
Error bars represent the standard deviation from individual patches from the top clones in the assay

### Example 2: Resting Cell Assay with OA or CBGA Feed and Varying pH

A colony from a plate patched with strain SB824 was grown in a shaker flask (40 mL) containing YPD. After 48 h of growth at 300 C, 0.5 mL of cell culture was added in 96 well plate. The cells were pelleted by centrifugation (4,000 rpm for 5 min) and the supernatant was decanted. The cell pellets were then resuspended in fresh YPD media containing 6% glucose and different buffering systems: 100 mM phthalate (Phth) at pH 4.5 or 5.5 or MES at pH 5.5 or 6.5. The cells at these varying pHs were then mixed with either 3 mM of OA or 3 mM of CBGA. Cells were inoculated in a high speed shaker at 30° C. for 3 days. The THCA and CBCA that was produced is shown in Table 5.

TABLE 5
THCA and CBCA formation from strain SB824
at different pH and OA or CBGA feeds

| | | Day 2 | | Day 3 | |
|---|---|---|---|---|---|
| | | THCA | | THCA | |
| Feed | pH | (mM) | CBCA | (mM) | CBCA |
| OA | 4.5-Phth | 0 | 0 | 63 | 0 |
| OA | 5.5-Phth | 193 | 0 | 376 | 9 |
| OA | 5.5-MES | 115 | 0 | 192 | 3 |
| OA | 6.5-MES | 65 | 12 | 140 | 21 |
| CBGA | 4.5-Phth | 62 | 0 | 131 | 0 |
| CBGA | 5.5-Phth | 134 | 2 | 324 | 7 |
| CBGA | 5.5-MES | 77 | 0 | 200 | 3 |
| CBGA | 6.5-MES | 89 | 8 | 163 | 13 |

When cells are fed OA, they quickly accumulated CBGA (results not shown) since this strain also contains CBGA synthase. As seen in Table 5 the pH of the media had a big effect in both THCA titer as well as the formation of CBCA byproduct, with pH=5.5 being the best. These results agree with literature reports of purified THAS where activity is reduced in pH above 6 and was accompanied by formation of CBCA. The results also show that THCAS is either secreted on the supernatant or is trapped in the outer cell membrane or periplasmic space and as a result its activity is influenced by the extracellular pH; if the enzyme was intracellular both its activity and selectivity would be unaffected at varying pHs.

### Example 3: Expression of THCAS in YL and Synthesis of THCVA from Butyric Acid Feed

Patched colonies were used to inoculate 0.5 mL YPD media in 96 DW blocks, which were grown in a high-speed shaker at 30° C. with the shake speed set to 1000 rpm. After 48 h, 20 μL from each preculture was used to inoculate another 96 DW plate pre-loaded with 0.5 mL of YPD media (2% glucose) with 100 mM MES pH 5.5+2.5 mM butyric acid, which were then grown at 30° C. with the shake speed set to 1000 rpm.

Cultures were supplemented with an additional 2% glucose and 5 mM butyric acid three times (after 24 h, 48 h, 72 h) by adding 20 μL into each well from a stock solution that contained: 50% glucose and 125 mM butyric acid. After 96 h of incubation the cultures were quenched with 0.5 mL ethanol with 0.1% formic acid and 0.1 mg/mL pentyl-benzoic acid and submitted for LC/MS analyses. Table 6 lists the highest concentration of THCVA produced among the different transformants screened of each strain with this butyric acid feed regimen.

TABLE 6
THCA formation from a butyric acid feed (C4-FFA).
Products in μM accumulated in the in vivo assay.
Butyric Acid (C4 FFA) Feed Assays

| | | | [THCVA], |
|---|---|---|---|
| Clone | | Enzyme | μM |
| # | Strain | Expressed | Produced ª |
| 1 | SB-0824 | SP3.BBE2.1 | 158 |
| 2 | SB-0824 | SP3.BBE2.1 | 122 |
| 3 | SB-0824 | SP3.BBE2.1 | 86 |
| 4 | SB-0824 | SP3.BBE2.1 | 64 |

^{a} Total THCVA produced after 96 hrs, with iterative 5 mM butyric acid feeds along with 2% glucose every 24 hrs for three days and an initial starting butyric acid concentration of 2.5 mM (17.5 mM butyric acid total).

### Example 4: Expression of THCAS in YL and Synthesis of THCVA from Butyric Acid or DVA Feed

In this example, THCAS was fused with a naturally secreted protein in Yarrowia, Lipase 2 (Lip2; SEQ ID NO: 100) or a protein that is attached in the outer membrane of the cell, cell wall protein CWP1 (SEQ ID NO: 103). The constructs were cloned in a plasmid (Table 7) and were tested for THCA formation. Plasmids pCL-SE-0772 and -0797 were linearized with AsiSI and PsilI. Plasmid pCL-SE-0801 was linearized with DraI. The linearized plasmids were transformed into strain SB-889 that can convert supplemental OA to CBGA as described in Example 1. Multiple clones per transformation were pre-cultured for 24 h in 500 μl YPD incubated in 96 deep well plates shaking at >900 RPM at 30° C. 2 μl of the preculture was used to inoculate 500 μl YPD supplemented with 100 mM MES pH 5.5 and 2 mM OA in YPD incubated in 96 deep well plates shaking at >900 RPM at 30° C. After 72 hours cultures were quenched and evaluated THCA. The results are shown in the tables below:

TABLE 7
THCA formation from OA feed using fused and unfused THCAS.
Products in μM accumulated in the in vivo assay.

| | Upstream pathway | Enzyme | |
|---|---|---|---|
| Strain | enzymes introduced | Expressed | THCA |
| SB-889(1) | HCS2, PKS1, | — | 0 |
| | PKC1.1, HMGR, | | |
| | ERG20ww, | | |
| | ERG20ww-PKC1.1- | | |
| | MPT4, ACS1, | | |
| | ACC1 | | |
| SB-0889 + | | THCAS | 37.2 +/− 5.2 |
| pCL-SE-0772 | | | |
| SB-0889 + | | Lip2-THCAS | 22.4 +/− 2.0 |
| pCL-SE-0797 | | | |
| SB-0889 + | | THCAS-CWP1 | 24.0 +/− 1.9 |
| pCL-SE-0801 | | | |

(1)This strain is described in more detail in commonly owned U.S. Provisional

Application No. 63/256,398, which is incorporated by reference in its entirety.

Results clearly show that although THCA titers were not increased, both fusions produced active THCA synthase. Further improvement of expression of these constructs by optimizing host cell as described herein, THCAS sequence and linker sequence will further improve THCA titers.

### Example 5: Modifications of Yarrrowia: Knock Out of Proteases

To examine one of the proteases of interest, YALI0F09163p, the gene encoding this protease was disrupted in an A28 CBGA producing strain, SB-1691, to generate SB-2702. The construction of SB-1691, and other A28 strains are described in more detail in commonly owned U.S. Provisional Application No. 63/256,398, which is incorporated herein by reference in its entirety. A plasmid, pCL-SE-0849, that expresses THCAS using the SP4 signal peptide was transformed into each strain and THCA production examined as using a hexanoic acid feed assay. Patched colonies were used to inoculate 0.5 mL YDCM001 (YNB+nitrogen 6.71 g/L, glucose 20 g/L, casamino acids 10 g/L, 100 mM MES (pH6.5)) media in 96dw blocks which were grown at 30° C. with 1000 rpm shaking. After 24 h, 2 μL from each preculture was used to inoculate 0.5 mL YDCM001 media. After another 24 h, 15 μL of 100 mM hexanoic acid was added and the cultures incubated for an additional 24 h at 30° C. with 1000 rpm shaking. Cultures were then quenched with 0.5 mL ethanol with 0.1 mg/mL pentyl-benzoic acid and submitted for LC analysis. Table 8 lists the concentration of THCA produced among the different transformants. As is evident, the strain (SB-2702) where YALI0F09163 was disrupted was able to produce more THCA than the control (SB-1961)

TABLE 8
THCA formation from hexanoic acid feed in strain with YALI0F09163
disrupted. Products in μM accumulated in the in vivo assay.

| | Upstream | | | |
|---|---|---|---|---|
| | pathway enzymes | | Enzyme | |
| Strain | introduced | disruptions | Expressed | THCA |
| SB-1691 + | HCS2, PKS1.1, | Δerg20(1) | SP4- | 13.8 ± 0.3 |
| pCL- | PKC1, ERG20ww, | | THCAS | |
| SE-0849 | ERG20ww- | | | |
| | PKC1.1-MPT4.1 | | | |
| SB-2072 + | HCS2, PKS1.1, | Δerg20, | SP4- | 145.5 ± 2.7 |
| pCL- | PKC1, ERG20ww, | ΔYALI0F09163 | THCAS | |
| SE-0849 | ERG20ww- | | | |
| | PKC1.1-MPT4.1 | | | |

(1)The construction of this strain is described in detail in commonly owned U.S.

Provisional Application 63/256,398, which is hereby incorporated by reference in its entirety.

Additional proteases of interest (Table 2) are inactivated in SB-1691, a strain that produces CBGA from hexanoic acid. These modified strains are transformed with a construct expressing THCAS using the SP4 signal peptide (pCL-SE-0849). As a control, SB-1691 is also transformed with the same constructs. THCA production is examined as above.

### Example 6: Modifications of Yarrowia: Expression of Chaperones

Strain, SB1008, expressing HCS2, PKS1, PKC1.1, HMGR, ERG20ww, ERG20ww-PKC1.1-MPT4, and THCAS using SP4 signal peptide is transformed with various vectors expressing chaperones (Table 9). Transformants were examined for THCA production as described in Example 4, with the modification that 1 g/L Hygromycin was added to the media. Results are shown in FIG. 8A. Of the initial list of chaperones, IRE overexpression results in no growth while HAC1s resulted in slow growth. To examine HAC1s, this gene was expressed with a weaker promoter, which allowed for growth consistent with the parent strain. The HAC1s transformants were examined for THCA production as above, but the media used was adjusted to pH of 6.0. The results are shown in FIG. 8B. Of all the chaperones tested, overexpression of CNE1, FADS and HAC1s provided the most robust improvement in THCA production.

TABLE 9
THCA formation from OA feed in THCA producing
strains overexpressing chaperones and other
accessory proteins and regulators.
Products in μM accumulated in the in vivo assay.

| Plasmid | Chaperone | THCA |
|---|---|---|
| empty vector | — | 17.4 ± 1.1 |
| pCL-SE-0713 | Yl.HAC1 | 41.6 ± 27 |
| pCL-SE-0715 | Yl.FADS1 | 83.9 ± 46.4 |
| pCL-SE-0716 | Yl.KAR2 | 29.9 ± 7.6 |
| pCL-SE-0717 | Yl.FMN1 | 21.2 ± 11.7 |
| pCL-SE-0718 | Yl.CNE1 | 168.1 ± 99.8 |
| pCL-SE-0719 | Yl.ERO1 | 10.8 ± 4.7 |
| pCL-SE-0720 | Yl.PDI1 | 8.1 ± 6.5 |
| pCL-SE-0722 | Yl.YAP1 | 18.5 ± 4.9 |
| pCL-SE-0723 | Yl.HYR1 | 24.5 ± 0.9 |
| pCL-SE-0724 | SP3-CsCHAP1.Yl1 | 22.9 ± 7.7 |
| pCL-SE-0725 | SP7-CsCHAP1.Yl1 | 25.2 ± 10.4 |
| pCL-SE-0726 | SP8-CsCHAP1.Yl1 | 24.1 ± 6.7 |
| pCL-SE-0727 | SP.KAR2-CsCHAP1.Yl1 | 32.2 ± 14.5 |
| pCL-SE-0728 | SP3-CsCHAP1.Yl1-HDEL | 18.3 ± 6.2 |
| pCL-SE-0729 | SP7-CsCHAP1.Yl1-HDEL | 23 ± 11.9 |
| pCL-SE-0730 | SP8-CsCHAP1.Yl1-HDEL | 35 ± 19.2 |
| pCL-SE-0731 | SP.KAR2-CsCHAP1.Yl1- | 22.3 ± 3.5 |
| | HDEL | |
| pCL-SE-0732 | SP3-CsCHAP2.Yl1 | 20.9 ± 11.1 |
| pCL-SE-0733 | SP7-CsCHAP2.Yl1 | 15.5 ± 7 |
| pCL-SE-0734 | SP8-CsCHAP2.Yl1 | 25.2 ± 5.8 |
| pCL-SE-0735 | SP.KAR2-CsCHAP2.Yl1 | 25.2 ± 8.1 |
| pCL-SE-0736 | SP3-CsCHAP2.Yl1-HDEL | 28.5 ± 19.5 |
| pCL-SE-0737 | SP7-CsCHAP2.Yl1-HDEL | 18 ± 1.4 |
| pCL-SE-0738 | SP8-CsCHAP2.Yl1-HDEL | 21.9 ± 4.7 |
| pCL-SE-0739 | SP.KAR2-CsCHAP2.Yl1- | 30 ± 17.2 |
| | HDEL | |

TABLE 10
THCA formation from OA
feed in THCA producing strains
overexpressing chaperones.
Products in μM
accumulated in the in vivo assay.

| Plasmid | Chaperone | THCA |
|---|---|---|
| — | — | 229.9 ± 6.1 |
| pCL- | Yl.HAC1- | 611.9 ± 44.5 |
| SE- | spliced | |
| 0860 | (weaker | |
| | promoter) | |

### Example 7: Modifications of Yarrowia: Modification of Glycosylation

The ROT2 glucosidase is inactivated in SB-691. This modified strain is transformed with a construct expressing THCAS using the SP3 signal peptide (pCL-SE-0703) or SP4 signal peptide (pCL-SE-0704). As a control, SB-691 is also transformed with the same constructs. THCA production is examined as described in Example 5.

### Example 8: Expression of THCAS/CBDAS Homologs from Plants, Fungi and Bacteria

The approach is taken to identify new enzymes for each step relies on three general methods. The first involves identifying sequence homologs to known enzymes with the desired activity. The second method relies on literature searches for enzymes that perform similar reactions using the same substrates or enzymes that perform the same reaction with similar substrates. The third method utilizes artificial intelligence algorithms to identify potential enzymes based on predicted activities. These methods identified many candidate sequences that were then manually curated and the selected sequences were cloned and characterized. In addition to natural sequences, several mutants were created using rational and random mutagenesis techniques. The mature sequences of all enzymes as shown BBE1.1-BBE58 were fused to a secretion sequence from SEQ ID NOS: 92-97. Synthetic genes optimized for expression in Yarrowia were made and their expression and activity towards CBGA cyclization was evaluated as described in Example 1.

Strain SB-1522-4.2, which is an A28 CBG(V)A producing strain expressing HCS2, PKS1.1, PKC1.0, ERG20ww, ERG20ww-MPT4, ERG20.A28 and CNE1 and carries a disruption of ERG20, was transformed with plasmids expressing BBE variants with SP4 signal peptide. Four colonies from each transformation were used to inoculate 0.5 mL YDCM (YNB+Nitrogen 6.71 g/L, casamino acids 10 g/L, glucose 60 g/L, 100 mM MES pH 6.5) media with 1 mg/mL hygromycin in 96dw blocks, which were grown at 30° C. with 1000 rpm shaking. After 48 h, 2 μL from each preculture was used to inoculate 0.5 mL YDCM media with 1 mg/mL hygromycin in duplicate 96dw blocks. Blocks were grown at 30° C. with 1000 rpm shaking. After 24 h, cultures were supplemented with 1 mM CBGA or CBGVA by adding 5 μL of 100 mM CBGA or CBGVA in 50% ethanol. After an additional 24 h (48 h total), cultures were quenched with 0.5 mL ethanol with 0.2 mg/mL pentyl-benzoic acid and submitted for LC analysis. For a more detailed description of A28 strains, see commonly owned U.S. Provisional Application 63/256,398, which is hereby incorporated by reference in its entirety.

TABLE 11
Cannabinoid formation from
CBGA using BBE
variants with SP4 signal
peptide. Products in μM
accumulated in the in vivo assay.
Error bars represent
the standard deviation
from individual replicates.

| BBE Expressed | CBDA | THCA | CBCA |
|---|---|---|---|
| SP4.BBE1.6 | 19 ± 1 | | |
| SP4.BBE1.20 | 30 ± 2 | | |
| SP4.BBE1.21 | 44 ± 4 | | |
| SP4.BBE1.22 | 31 ± 3 | | 8 ± 1 |
| SP4.BBE2.1 | | 75 ± 5 | |
| SP4.BBE2.6 | | 92 ± 11 | |
| SP4.BBE2.7 | | 102 ± 2 | |
| SP4.BBE2.8 | | 102 ± 13 | |
| SP4.BBE2.16 | | 108 ± 9 | |
| SP4.BBE2.18 | | 93 ± 9 | |
| SP4.BBE2.19 | | 68 ± 7 | |
| SP4.BBE2.20 | | 121 ± 17 | |
| SP4.BBE2.21 | | 80 ± 14 | |
| SP4.BBE2.22 | | 100 ± 2 | |
| SP4.BBE3.1 | | | 0 ± 0 |
| SP4.BBE2.14 | | | 4 ± 0 |
| SP4.BBE25.1 | | | 28 ± 8 |
| SP4.BBE25.4 | | | 11 ± 3 |
| SP4.BBE25.5 | | | 56 ± 18 |

TABLE 12
Cannabinoid formation from CBGVA using BBE
variants with SP4 signal peptide. Products in μM
accumulated in the in vivo assay. Error bars represent
the standard deviation from individual replicates.

| BBE Expressed | CBDVA | THCVA | CBCVA |
|---|---|---|---|
| SP4.BBE1.6 | 157 ± 38 | 6 ± 4 | 25 ± 3 |
| SP4.BBE1.20 | 209 ± 45 | 17 ± 3 | 26 ± 7 |
| SP4.BBE1.21 | 245 ± 20 | 18 ± 2 | 41 ± 5 |
| SP4.BBE1.22 | 106 ± 7 | 11 ± 1 | 29 ± 2 |
| SP4.BBE2.1 | | 244 ± 17 | |
| SP4.BBE2.6 | | 400 ± 55 | |
| SP4.BBE2.7 | | 298 ± 52 | |
| SP4.BBE2.8 | | 556 ± 94 | |
| SP4.BBE2.16 | | 398 ± 52 | |
| SP4.BBE2.18 | | 390 ± 59 | |
| SP4.BBE2.19 | | 270 ± 50 | |
| SP4.BBE2.20 | | 375 ± 47 | |
| SP4.BBE2.21 | | ND | |
| SP4.BBE2.22 | | 331 ± 35 | |
| SP4.BBE3.1 | | | 0 ± 0 |
| SP4.BBE2.14 | | | 23 ± 4 |
| SP4.BBE25.1 | | | 252 ± 45 |
| SP4.BBE25.4 | | | 99 ± 23 |
| SP4.BBE25.5 | | | 543 ± 64 |

ND: the cultures expressing SP4.BBE2.21 did not receive CBGVA.

Results clearly show that mutations in BBE1.20, BBE1.21, and BBE1.22 improve CBDA and CBDVA (except for BBE1.22) production compared to the WT enzyme, BBE1.6. Similarly, mutations in BBE 2.6, BBE2.7, BBE2.8, BBE2.16, BBE2.18, BBE2.19, BBE2.20, BBE2.21, and BBE2.22 improve THCA (except for BBE2.19) and THCVA (except for BBE2.21 which was not tested with CBGVA) production compared to the WT enzyme, BBE2.1. Mutations in BBE2.14 change the enzyme's product profile compared to the WT enzyme, BBE2.1 and completely convert the enzyme from a THCA- and THCVA-producing enzyme into a CBCA- and CBCVA-producing enzyme. BBE25.1, BBE25.4, and BBE25.5 are natural sequences that have not been characterized but are annotated as cannabidiolic acid synthase-like. However, the results in tables 11-12 clearly show that these enzymes produce CBCA and CBCVA, whereas the WT CBCAS enzyme, BBE3.1, produces no product in our system.

### Methods and Techniques

### Analytical Methods

All samples after quenching with equal volume of EtOH were centrifuged and were analyzed by HPLC-MS
Column: 2.1×50 mm COSMOCORE PBr (Nacalai USA, Inc.)
Buffers:
Buffer A: Water, 0.1% formic acid
Buffer B: Acetonitrile, 0.1% formic acid
Flow rate: 0.45 mL/min
Column Temperature: 50 Celsius
Injection vol.: 2 uL
Gradient Method:

| Time | Buffer B % |
|---|---|
| 0 | 20 |
| 1.5 | 50 |
| 7.5 | 77 |
| 7.6 | 20 |
| 9.5 | 20 |

Retention times: OA 2.49 min, OL 2.38 min, CBGVA 4.71, CBDA 5.2 min, CBGA 5.60 min, THCVA 4.71 min, THCA 7.1 min, CBCA 7.31 min, and F-CBGA 7.52 min.

All compounds were verified by comparing with authentic standards and/or analyzing by LC-MS

Process Development for Making Cannabinoids through Fermentation

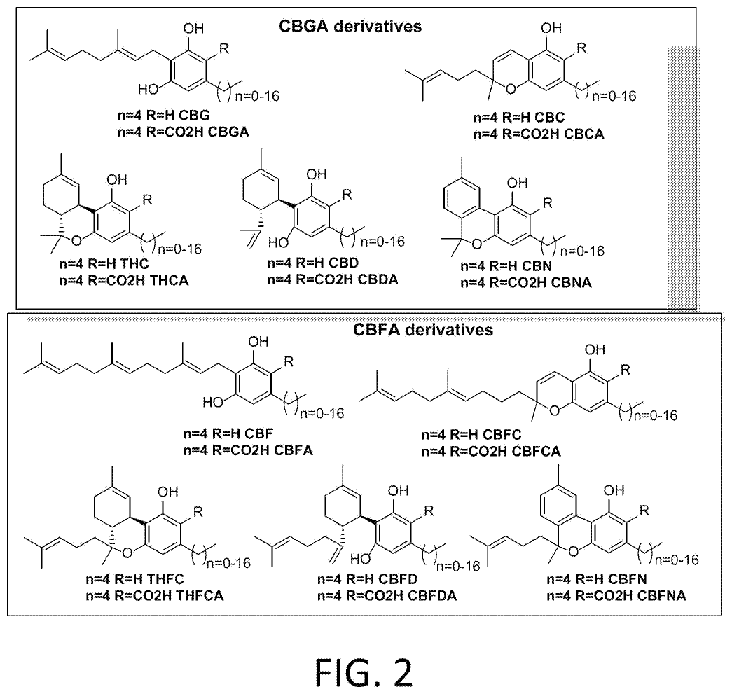

The above CBGA synthases can be used in cell free reactions (in vitro) or whole cell biotransformations to produce cannabinoids as described in FIG. 2. Recombinant cells of yeast, bacteria, fungi, algae, or plant will express the terminal synthase (THCAS, CBDAS, CBCAS, etc.) and be contacted in appropriate fermentation/reaction conditions with CBGA, FCBGA or other CBGA to produce the final cannabinoid. These organisms will also contain genes encoding (a) proteins capable of converting different acyl-CoAs (Acetyl, butyryl, Hexanoyl, etc) into the corresponding OA derivatives (orselinic acid, DVA, OA etc) and (b) an appropriate prenyl transferase capable of prenylating them to the CBGA derivatives shown in FIG. 2.

Genes for OA synthesis may include one or more of the following: acyl-CoA ligase/synthase, polyketide/tetraketide synthases and polyketide cyclases, and prenyl transferase. The later can be membrane bound such as PT4 (Uniprot AOA455ZJC3), soluble such as nphB (Q4R2T2) or other enzymes with prenylation activity including enzyme fusions and mutants as described in WO 2021/178976, published Sep. 10, 2021, incorporated herein by reference. Genes that increase mevalonate or MEP pathway flux towards GPP or FPP formation will also be overexpressed in the previous organism. Increasing the intracellular concentration of GPP, mutant farnesyl pyrophosphate synthases may be used as have been described in yeast (Jian G-Z, et al Metabolic Engineering, 2017, 41, 57) or GPP specific synthases can be introduced (Schmidt A, Gershenzon J. Phytochemistry, 2008, 69, 49). Other enzymes in the mevalonate pathway (for example HMG-CoA reductase) may need to be manipulated (truncated or mutated) or be overexpressed. The formation of cannabinoid products can occur when the organism is grown with simple carbon sources, such as glucose, sucrose, glycerol, or another simple or complex sugar mixture. External organic acids with carbon chains varying from 4 to more than 12 (in straight or branched chains) can also be supplemented during growth.

For scaling the production of these molecules, cells will then be grown in stirred tank fermenters with feed supplementation (sugars with or without organic acids) where the dissolved oxygen, temperature, and pH will be controlled according to the optimal growth and production process. Addition of aqueous non-miscible organic solvents to dissolve added organic acids or extract the cannabinoid products as they are being synthesized may also be used. These solvents may include, but are not limited to, isopropyl myristate (IPM), diisobutyl adipate, decane, dodecane, hexadecane or anther organic solvent with log P>5.

Depending on the fermentation process, the products can be isolated and purified using different methods. If no organic cosolvent is used the cannabinoid will be insoluble in the fermentation broth and precipitates together with the cells after centrifugation. In such case, the cell paste (wet, heat dried, lyophilized or spray dried) is used for isolation. Methods commonly used in the isolation and decarboxylation of cannabinoids can be applied to this material. These methods usually consist of two an extraction with supercritical CO2 or using an organic solvent most commonly ethanol. After extraction, the ethanol mixture can be “winterized” or be incubated at −40 to −50 C to precipitate oils and waxes followed by the evaporation of ethanol to produce a cannabinoid containing solid or oil. Final purification steps will then include fractional distillation, crystallization, centrifugal partition chromatography, or a combination of these methods.

An organic solvent (such as IPM, dodecane, etc) can be used during fermentation to continuous extract the cannabinoid products and eliminate any possible toxicity. At the end of the fermentation the mixture will be centrifuged and the organic solvent will be separated. The cannabinoid acids will then be extracted to the aqueous phase using alkaline water basic aqueous solvent will extract the cannabinoids to the aqueous phase. Acidification of the aqueous solution will precipitate the cannabinoids that can be isolated by filtration. Further isolation may involve back extraction of the acidified aqueous solution with low boiling point organic solvent (e.g., ethyl acetate, hexane, etc.) evaporation of the organic solvent will produce solid cannabinoid that can be further purified by fractional distillation or recrystallization.

In some embodiments, Cannabinoids in this application are defined as products that are produced from reacting olivetolic acid and its analogs with GPP or FPP as shown in FIG. 2. Cannabinoids are also defined as the cyclization products of the previous CBGA analogs to produce CBDA, THCA and CBCA analogs in addition to producing other novel cyclization products. Some examples of these analogs are shown in FIG. 1.

SEQUENCE LISTING

>BBE1.1
NPRENFLKCFSQYIPNNATNLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHRH (SEQ ID NO: 1).
>BBE1.2
NPRENFLKCFSQYIPNNATNLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHHH (SEQ ID NO: 2).
>BBE1.3
NPRENFLKCFSQYIPNNATNLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVGVGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHRH (SEQ ID NO: 3).
>BBE1.4
NPRENFLKCFSQYIPNNATNLKLVYTQNNPLYMSVLNSTIQNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVGVGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHRH (SEQ ID NO: 4).
>BBE1.5
NPRENFLKCFSKHIPNNVANPKLVYTQHDQLYMSILNSTIQNLRFISDT
TPKPLVIVTPSNNSHIQATILCSKKVGLQIRTRSGGHDAEGMSYISQVPFVVVDLRNM
RSIKIDVHSQTAWVEAGATLGEVYYWINEKNENLSFPGGYCPTVGVGGHFSGGGYG
ALMRNYGLAADNIIDAHLVNVHGKVLDRKSMGEDLFWALRGGGAESFGIIVAWKIR
LVAVPKSTMFSVKKIMEIHELVKLVNKWQNIAYKYDKDLLLMTHFITRNITDNQGKN
KTAIHTYFSSVFLGGVDSLVDLMNKSFPELGIKKTDCRQLSWIDTIIFYSGVVNYDTD
NFNKEILLDRSAGQNGAFKIKLDYVKKPIPESVFVQILEKLYEEDIGAGMYALYPYGG
IMDEISESAIPFPHRAGILYELWYICSWEKQEDNEKHLNWIRNIYNFMTPYVSKNPRL
AYLNYRDLDIGINDPKNPNNYTQARIWGEKYFGKNFNRLVKVKTKVDPNNFFRNEQ
SIPPLPPHRH (SEQ ID NO: 5).
>BBE1.7
NPRENFLKCFSQYIPNNVANLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHRH (SEQ ID NO: 6).
>BBE1.8
NPRENFLKCFSQYIPNNATNLKLVYTQNNPLYMSVLNSTIHNLRFTSD
TTPKPLVIVTPSNVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHRH (SEQ ID NO: 7).
>BBE1.9
NPRENFLKCFSQYIPNNATNLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFTTPYVSKNPRL
AYLNYRDLDIGINDPKNPNNYTQARIWGEKYFGKNFDRLVKVKTLVDPNNFFRNEQ
SIPPLPRHRH (SEQ ID NO: 8).
>BBE1.10
NPRENFLKCFSQYIPNNVANLKLVYTQNNPLYMSVLNSTIHNLRFTSD
TTPKPLVIVTPSNVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHRH (SEQ ID NO: 9).
>BBE1.11
NPRENFLKCFSQYIPNNVANLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFTTPYVSKNPRL
AYLNYRDLDIGINDPKNPNNYTQARIWGEKYFGKNFDRLVKVKTLVDPNNFFRNEQ
SIPPLPRHRH (SEQ ID NO: 10).
>BBE1.12
NPRENFLKCFSQYIPNNATNLKLVYTQNNPLYMSVLNSTIHNLRFTSD
TTPKPLVIVTPSNVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFTTPYVSKNPRL
AYLNYRDLDIGINDPKNPNNYTQARIWGEKYFGKNFDRLVKVKTLVDPNNFFRNEQ
SIPPLPRHRH (SEQ ID NO: 11).
>BBE1.13
NPRENFLKCFSQYIPNNVANLKLVYTQNNPLYMSVLNSTIHNLRFTSD
TTPKPLVIVTPSNVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFTTPYVSKNPRL
AYLNYRDLDIGINDPKNPNNYTQARIWGEKYFGKNFDRLVKVKTLVDPNNFFRNEQ
SIPPLPRHRH (SEQ ID NO: 12).
>BBE1.14
NPRENFLKCFSQYIPNNATNLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDCKNPNNYTQARIWGEKYFGKNFDRLVKVKTLVDPNNFFCNE
QSIPPLPRHRH (SEQ ID NO: 13).
>BBE1.15
NPRENFLKCFSQYIPNNATNLKLVYTQNNPLYMSVLNSTIHNLRFTSD
TTPKPLVIVTPSHVSHIQGTILCSKKVGLQIRTRSGGHDAEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHRH (SEQ ID NO: 14).
>BBE1.16
NPRENFLKCFSQYIPNNATNLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYVLYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHRH (SEQ ID NO: 15).
>BBE1.17
NPRENFLKCFSQYIPNNATNLKLVYTQNNPLYMSVLNSTIHNLRFSSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNESLSLAAGYCPTVCAGGHFGGGGY
GPLMRSYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSQNPR
LAYLNYRDLDIGINDPKNPNNYTQARIWGEKYFGKNFDRLVKVKTLVDPNNFFRNE
QSIPPLPRHRH (SEQ ID NO: 16).
>BBE1.18
NPRENFLKCFSQYIPNNATNLKLVYTQNNPLYMSVLNSTIHNLRFSSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNESLSLAAGYCPTVCAGGHFGGGGY
GPLMRSYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSARQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSQNPR
LAYLNYRDLDIGINDPKNPNNYTQARIWGEKYFGKNFDRLVKVKTLVDPNNFFRNE
QSIPPLPRHRH (SEQ ID NO: 17).
>BBE1.19
NPRENFLKCFSQYIPNNATNLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNSR
LAYLNYRDLDIGINDPKNPNNYTQARIWGEKYFGKNFDRLVKVKTLVDPNNFFRNE
QSIPPLPRHRH (SEQ ID NO: 18).
>BBE1.20
NPRENFLKCFSQYIPNNATNLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITKNITDNQGK
NKTAIHTYFSSVFLGGVDSLVDLMNKSFPELGIKKTDCRQLSWIDTIIFYSGVVNFDTD
NFNKEILLDRSAGQNGAFKIKLDYVKKPIPESVFVQILEKLYEEDIGAGMYALYPYGG
IMEEISESAIPFPHRAGILYELWYICSWEKQEDNEKHLNWIRNIYNFMTPYVSKNPRLA
YLNYRDLDIGINDPKNPNNYTQARIWGEKYFGKNFDRLVKVKTLVDPNNFFRNEQSI
PPLPPHHH (SEQ ID NO: 19).
>BBE2.1
NPRENFLKCFSKHIPNNVANPKLVYTQHDQLYMSILNSTIQNLRFISDT
TPKPLVIVTPSNNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFTTPYVSQN
PRLAYLNYRDLDLGKTNHASPNNYTQARIWGEKYFGKNFNRLVKVKTKVDPNNFFR
NEQSIPPLPPHHH (SEQ ID NO: 20).
>BBE2.2
NPRENFLKCFSKHIPNNATNPKLVYTQHDQLYMSILNSTIQNLRFISDT
TPKPLVIVTPSNNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNENT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFTTPYVSQN
PRLAYLNYRDLDLGKTNHASPNNYTQARIWGEKYFGKNFNRLVKVKTKVDPNNFFR
NEQSIPPLPPHHH (SEQ ID NO: 21).
>BBE2.3
NPRENFLKCFSKHIPNNVANPKLVYTQHDQLYMSILNSTIQNLRFISDT
TPKPLVIVTPSQNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFTTPYVSQN
PRLAYLNYRDLDLGKTNHASPNNYTQARIWGEKYFGKNFNRLVKVKTKVDPNNFFR
NEQSIPPLPPHHH (SEQ ID NO: 22).
>BBE2.4
NPRENFLKCFSKHIPNNVANPKLVYTQHDQLYMSILNSTIQNLRFISDT
TPKPLVIVTPSNNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFMTPYVSQN
PRLAYLNYRDLDLGKTNHASPNNYTQARIWGEKYFGKNFNRLVKVKTKVDPNNFFR
NEQSIPPLPPHHH (SEQ ID NO: 23).
>BBE2.5
NPRENFLKCFSKHIPNNATNPKLVYTQHDQLYMSILNSTIQNLRFISDT
TPKPLVIVTPSQNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFTTPYVSQN
PRLAYLNYRDLDLGKTNHASPNNYTQARIWGEKYFGKNFNRLVKVKTKVDPNNFFR
NEQSIPPLPPHHH (SEQ ID NO: 24).
>BBE2.6
NPRENFLKCFSKHIPNNATNPKLVYTQHDQLYMSILNSTIQNLRFISDT
TPKPLVIVTPSNNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNENT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFMTPYVSQN
PRLAYLNYRDLDLGKTNHASPNNYTQARIWGEKYFGKNFNRLVKVKTKVDPNNFFR
NEQSIPPLPPHHH (SEQ ID NO: 25).
>BBE2.7
NPRENFLKCFSKHIPNNVANPKLVYTQHDQLYMSILNSTIQNLRFISDT
TPKPLVIVTPSQNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFMTPYVSQN
PRLAYLNYRDLDLGKTNHASPNNYTQARIWGEKYFGKNFNRLVKVKTKVDPNNFFR
NEQSIPPLPPHHH (SEQ ID NO: 26).
>BBE2.8
NPRENFLKCFSKHIPNNATNPKLVYTQHDQLYMSILNSTIQNLRFISDT
TPKPLVIVTPSQNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFMTPYVSQN
PRLAYLNYRDLDLGKTNHASPNNYTQARIWGEKYFGKNFNRLVKVKTKVDPNNFFR
NEQSIPPLPPHHH (SEQ ID NO: 27).
>BBE2.9
NPRENFLKCFSKHIPNNVANPKLVYTQHDQLYMSILNSTIQNLRFISDT
TPKPLVIVTPSNNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFTTPYVSQN
PRLAYLNYRDLDLGKTNHASPNQYTQARIWGEKYFGKNFNRLVKVKTKVDPNNFFR
NEQSIPPLPPHHH (SEQ ID NO: 28).
>BBE2.10
NPRENFLKCFSKHIPNNVANPKLVYTQHDQLYMSILNSTIQNLRFISDT
TPKPLVIVTPSQNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFTTPYVSQN
PRLAYLNYRDLDLGKTNHASPNQYTQARIWGEKYFGKNFNRLVKVKTKVDPNNFFR
NEQSIPPLPPHHH (SEQ ID NO: 29).
>BBE2.11
NPRENFLKCFSKHIPNNVANPKLVYTQHDQLYMSILNSTIHNLRFISDT
TPKPLVIVTPSNNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNENT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFTTPYVSQN
PRLAYLNYRDLDLGKTNHASPNNYTQARIWGEKYFGKNFNRLVKVKTKVDPNNFFR
NEQSIPPLPPHHH (SEQ ID NO: 30).
>BBE2.12
NPRENFLKCFSKHIPNNVANPKLVYTQHDQLYMSILNSTIQNLRFISDT
TPKPLVIVTPSNNSHIQATILCSKKVGLQIRTRSGGHDAEGMSYISQLPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVEAGMYVLYPY
GGIMEEISESAIPFPHRAGIMYELWYTASWEKQEDNEKHINWVRSVYNFTTPYVSQN
PRLAYLNYRDLDLGKTNHASPNNYTQARIWGEKYFGKNFNRLVKVKTKVDPNNFFR
NEQSIPPLPPHHH (SEQ ID NO: 31).
>BBE2.13
NPRENFLKCFSKHIPNNVANPKLVYTQHDQLYMSILNSTIQNLRFISDT
TPKPLVIVTPSNNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGQNGAFKIKLDYVKKPIPETAMVKILEKLYEEDVGAGMYVLYPY
GGIMEEISESAIPFPHRAGIMYELWYTASWEKQEDNEKHINWVRSVYNFTTPYVSQN
PRLAYLNYRDLDLGKTNHASPNNYTQARIWGEKYFGKNFNRLVKVKTKVDPNNFFR
NEQSIPPLPPHHH (SEQ ID NO: 32).
>BBE2.14
NPRENFLKCFSKHIPNNVANPKLVYTQHDQLYMSILNSTIQNLRFISDT
TPKPLVIVTPSNNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLTTHFITKNITDNHGK
NKTTVHGYFSSIFHGGVDSLVDLMNKSFPELGIKKTDCKEFSWIDTTIFYSGVVNENT
ANFKKEILLDRSAGKKTAFSIKLDYVKKPIPETAMVKILEKLYEEDVGAGMYVLYPY
GGIMEEISESAIPFPHRAGIMYELWYTATWEKQEDNEKHINWVRSVYNFTTPYVSQN
PRLAYLNYRDLDLGKTNHASPNNYTQARIWGEKYFGKNFNRLVKVKTKVDPNNFFR
NEQSIPPLPPHHH (SEQ ID NO: 33).
>BBE3.1
NPQENFLKCFSEYIPNNPANPKFIYTQHDQLYMSVLNSTIQNLRFTSDT
TPKPLVIVTPSNVSHIQASILCSKKVGLQIRTRSGGHDAEGLSYISQVPFAIVDLRNMH
TVKVDIHSQTAWVEAGATLGEVYYWINEMNENFSFPGGYCPTVGVGGHFSGGGYG
ALMRNYGLAADNIIDAHLVNVDGKVLDRKSMGEDLFWAIRGGGGENFGIIAACKIK
LVVVPSKATIFSVKKNMEIHGLVKLFNKWQNIAYKYDKDLMLTTHFRTRNITDNHG
KNKTTVHGYFSSIFLGGVDSLVDLMNKSFPELGIKKTDCKELSWIDTTIFYSGVVNYN
TANFKKEILLDRSAGKKTAFSIKLDYVKKLIPETAMVKILEKLYEEEVGVGMYVLYP
YGGIMDEISESAIPFPHRAGIMYELWYTATWEKQEDNEKHINWVRSVYNFTTPYVSQ
NPRLAYLNYRDLDLGKTNPESPNNYTQARIWGEKYFGKNFNRLVKVKTKADPNNFF
RNEQSIPPLPPRHH (SEQ ID NO: 34).
>BBE3.2
NPQENFLKCFSEYIPNNPANPKFIYTQHDQLYMSVLNSTIQNLRFTSDT
TPKPLVIVTPSNVSHIQASILCSKKVGLQIRTRSGGHDAEGMSYISQVPFVVVDLRNM
HSIKIDVHSQTAWVEAGATLGEVYYWINEKNENFSFPGGYCPTVGVGGHFSGGGYG
ALMRNYGLAADNIIDAHLVNVDGKVLDRKSMGEDLFWAIRGGGGENFGIIAAWKIK
LVVVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGVGMYVLYPY
GGIMEEISESAIPFPHRAGIMYELWYTASWEKQEDNEKHINWVRSVYNFTTPYVSQN
PRLAYLNYRDLDLGKTNPESPNNYTQARIWGEKYFGKNFNRLVKVKTKADPNNFFR
NEQSIPPLPPRHH (SEQ ID NO: 35).
>BBE3.3
NPQENFLKCFSEYIPNNPANPKFIYTQHDQLYMSVLNSTIQNLRFTSDT
TPKPLVIVTPSNVSHIQASILCSKKVGLQIRTRSGGHDAEGLSYISQVPFAIVDLRNMH
TVKVDIHSQTAWVEAGATLGEVYYWINEMNENFSFPGGYCPTVGVGGHFSGGGYG
ALMRNYGLAADNIIDAHLVNVDGKVLDRKSMGEDLFWAIRGGGGENFGIIAAWKIK
LVVVPSKATIFSVKKNMEIHGLVKLFNKWQNIAYKYDKDLMLTTHFRTRNITDNHG
KNKTTVHGYFSSIFLGGVDSLVDLMNKSFPELGIKKTDSKELSWIDTTIFYSGVVNYN
TANFKKEILLDGSAGKKTAFSIKLDYVKKLIPETAMVKILEKLYEEEVGVGMYVLYP
YGGIMDEISESAIPFPHRVGIMYELWYTATWEKQEDNEKHINWVRSVYNFTTPYVSQ
NPRLAYLNYRDLDLGKTNPESPNNYTQARIWGEKYFGKNFNRLVKVKTKADPNNFF
RNEQSIPPLPPHHH (SEQ ID NO: 36).
>BBE3.4
NPQENFLKCFSEYIPNNPANPKFIYTQHDQLYMSVLNSTIQNLRFTSDT
TPKPLVIVTPSNVSHIQASILCSKKVGLQIRTRSGGHDAEGLSYISQVPFAIVDLRNMH
TVKVDIHSQTAWVEAGATLGEVYYWINEMNENFSFPGGYCPTVGVGGHFSGGGYG
ALMRNYGLAADNIIDAHLVNVDGKVLDRKSMGEDLFWAIRGGGGENFGIIAAWKIK
LVVVPSKATIFSVKKNMEIHGLVKLFNKWQNIAYKYDKDLMLTTHFRTRNITDNHG
KNKTTVHGYFSSIFLGGVDSLVDLMNKSFPELGIKKTDCKELSWIDTTIFYSGVVNYN
TANFKKEILLDRSAGKKTAFSIKLDYVKKLIPETAMVKILEKLYEEEVGVGMYVLYP
YDGIMDEISESAIPFPHRAGIMYELWYTATWEKQEDNEKHINWVRSVYNFTTPCVSQ
NPRLAYLNYRDLDLGKSNPESPNNYTQARIWGEKYFGKNFNRLVKVKTKADPNNFF
RNEQSIPPLPPRHH (SEQ ID NO: 37).
>BBE4.1
HENFLQCLFLHFPNSNSISKVIYTPYNSSYESIWLSEIRMDRFKPPAPIT
KPLAIVKPLEDSHIQTSIYCSKRHDVQIRIRSGGHDYEGLSYISYEAPFIIVDLENFQSITI
DVENSTAWVQAGATIGQLYYTIAQKSPTLGFSAGICPTVGVGGHFSGGGYGMMSRN
YGISADNIIDARLIDVNGRILDRKSMGEDLFWAIRGGGSASFGVIIAWHIKLLQVPETV
TVFTVNRTLEQNATKIVHRWQSVSAKIDEKLALRLYLRRANSSQDGKETGQASFVTL
YLGGVDTLLPLMEQSFPELGLARSDCIEMSWIDSILYFASLSGEPLDVLLNRTPSPYGL
YYKGKLDYVTEPISETGLESIWKRYLEYPEEMNELELSPYGGRLSEISKSATPFPNRAG
TLYSIHYAVYWAEAGKSALNRHISGIRRFYSFMTPYVTKNPRSTYVNCRDLDLGVNN
KGNTTSYEQASIWGLKYYGINFKRLVHVKTKVDPGNFFRNEQSIPPL (SEQ ID NO:
38).
>BBE5.1
NLASHNFLKCFHSQTNLSVDSNSSSSSNVIFTTNGSSYTSVLQSSAQNL
RFATASTPKPQLIVRPLHESHIQAAVICSRKNSINIRIRSGGHDYEGLSYVSNIPFVIIDL
ANLRSIKIDVENKTAWVESGATLGELYHAIAEESNTYGFPAGSCPTMGVGGHLGGGG
FGTIFRKYGLAADNVIDARIVDVNGRIFDRKSTGEELFWAIRGGGGASFGIIFAWKINL
VPVPSIVTVFDIGKTLEPAGATKLLQKWQLVADKLSDTLFLDVVIAGNSNNTIAASFR
ALFLGVSEELLSTMEESFPELGVERKDCTEMSWIQSVLYFAGFSTRESYDVLLNRTNP
YKSSFKAKSDYVKEPISEKGLQGLWKILGEEESGYMIWTPYGGRMSEISEFEIPFPHRS
GNLYLIQYMVTWEAEEETEEHVNWITRLYEYITPFVSKNPRSAYLNYRDLDLGINMN
ENESYEQASVWGLKYFNSNFKRLAQIKAEVDPSDFFWYEQSIPLFLS (SEQ ID NO:
39).
>BBE6.1
DSSYQTFLQCLKTKSPPQSPEISSILYDPTNSSFQTVLQSYVRNRRFNR
STTPKPLLIVTPKTETHVSYAVLCAQAINIQLKIRSGGHDYDGISYVSDQPFIILDMENL
RSISIDLPTQTAWVQAGAQLGELYYQISTKSKTLGFPAGVCPTVGVGGHLSGGGYGN
MLRKYGLSVDHVTDARIVDVKGRILDRKAMGEDLFWAIRGGGGASFGVVLAYKIGL
VPVPETVTVFRVERTLAQNATEVVYRWQYVADKIDNGLFIRLLLQPVTVNKTRTIRA
SFIGEFLGDCVKLMEVVGNGFPELGFKKEDCMEMSWIGSVLYWANFDNDTKPEALL
SRTPDSVNFGKRKSDYVQNPISKDGLEWIWKKMIEVGKTGFVFNPYGGRMSEIPAGE
TPFPHRAGNIFKMQYSVNWNEEGVDADKDYINQIRRLYSYMTPFVSKSPRGAYLNY
RDLDIGTSANGKNSYEEGKVYGEKYFMGNYARLVKVKTMVDPGNFFRNEQSIPPFLS
(SEQ ID NO: 40).
>BBE7.1
RIHDYQYPKTSFLQCLSHHSSSNSSNNDLTKVVYTTTNSSYFSVLNFTI
INPRESSPSTPKPLFIITPLYESQVQAAVICARKHGVQIRVRSGGHDYEGLSYVSEIPFIV
IDMINFRSITVDVERRTGWVQTGATLGELYYEINKKSKTLAFPGGACPTIGVGGHITG
GGYGAIFRKYGLAADNVIDAQLVDVEGRVLDRETMGEELFWAIRGGGGASFGVILA
WKVLLVRVPETVTVFSINRNLEHNLTKLVHRWQYIAHKLPKKLLLAVRFQTVNSTK
KGIEMKKLQATFISVFLGRVDGLLYLMEKRFPELGLVREDCTQMSWIQSALFVAGLP
SEQSPEILLDRTPQSRLSFKAKSDYVKEPIPEKGLEGIWERLYEEEVGSGIVIMSPYGG
KMSEIPESELPFGHRGGNMYKIQYLIYWEEEENSGIVVEKHISWIRRLYNYMTPFVSK
NPRSAYINYRDLDIGRNSNKGTATYAQASIWGVKYFGQNNFNRLVHVKTMVDPTNF
FRNEQSIPPLP (SEQ ID NO: 41).
>BBE8.1
NPHNNFLQCFSKHISNNNTSLAKLIHTPNDFSYISLLNSTIQNLRFTSPT
PKPLVIITPSNTSHVQACVLCSKKYGLQIRTRSGGHDFEGVSYVSKVPFVILDMRNLRS
ITIDVDNKTAWVESGATLGELYYRIAEKNENLSFPGGYCHSVGVGGHFSGGGYGAL
MRKYGLAADNVIDAHLVNADGEFVDRKSMGEDLFWAIRGGGGASFGIVLAWKIRL
VPVPSKVTILSVSKNLPINETVKIYNKWQNIAHKFDQDLLMVVRFLTVNSTDEHGKN
MTTIQATFFSIFLGRVDKFLSLMQTNFPELGVVRKDCFETSWIEMIFFFNEFSSEDKLE
VLLDPTNVVKGYFKGKLDYVRKPISEIVMVKLLEKLYEEDVGLAYIQMYPYGGKMS
EIPESAIPFPHRAGVMYKILYWSQWEKEEESERHMNWVRSVYNYMTPYVSKNPRAS
YINYRDLDLGTNNEKGPISYEQASIWGKKYFNKNFKKLVQVKTKVDPTNFFRNEQSI
PPSFPRLL (SEQ ID NO: 42).
>BBE9.1
NPHDNFLQCFSKHINNNNNKSIVKLIHTPNDPSYISVLNSTIQNLRFAS
PSTPKPLVIITPSNTSHVQACVLCSKKYGLQIRTRSGGHDFEGASYVSKVPFVILDMRN
LRSITVDVDTKTAWVEAGATIGELYYRIAEKNGNLSFPAGYCRTVGVGGHFSGGGY
GALLRKYGLAADNIIDAHLVNADGEFLDRKSMGEDLFWAIRGGGGASFGIILAWKIR
LVAVPSKVTMFSVSKNLEMNETVKIYNKWQNTAYKFDKDLLLFVSFMTINSTDSQG
KYKTTIQASFSSIFLGRVESLLILMQKKFPELGIERKDCLEKSWIETVVYFDGFSSGDTP
ESLLNTTFQQNVFFKVKLDYVKKPVPEVVMVKLLEKLYEEDVGVGFLMMYPYGGK
MDEISESAIPFPHRAGFMYKILYLSAWEKEGESEKHMNWVRSAYNFMSPYVSQNPRA
TYLNYRDLDLGTNNEKGPISYSQASVWGKKYFGMNFKRLVNVKTKVDPSNFFRNEQ
SIPPLLSRRL (SEQ ID NO: 43).
>BBE10.1
SCLCSTAAQACSILASTLPAEAFVAPNSTGYQKVQTSYWSETAGDLR
PACITRPGSTEQVASIVKVLSTCGSDVRFAIKSGGHGSWPGWSSTEGGVLISLDLIDGV
QNVADKGYAVVGSGARWVDVYKSLEPQGVTVIGGRFASIGVGGLIVGGGISYFTGL
HGMACDNVLNYEVVLADGTIANVNRTSKPDLFRALKGGGNQFGIVTRFSLKTYKQT
TQVWAGILTFSIDKASQVFNATQAFIDRYDPLSHMYLSMGGGSNPQLSFINAYVFYD
ASSPPAGEANPFAAFYAAKPIADTTSTQNYSALIASNNNGNTPQQRWLIGGHTFPNLV
APYGADLYLKQWQAFRDMANATAAGDQALLESMALVPVPARAMRAGENVTGVKN
VLGLEPDAGDAVWIDYTLAWLEAKDDGKMFEFVERISKAGDKLATSFAPGVASTNA
MQGAVTAKEAGFYRTNPRYMNYALLDQPVQSSYGKENEQFLQRVQKRYDPDGLW
RKNGGFLWG (SEQ ID NO: 44).
>BBE11.1
KPISQNGANDGLRALTKRTVSPDGSCGGSNTYTCPTGQCCSQYGYCG
TTTAYCGTGCQSGFGTCSGSTPPTSTSTSTPSSATPTSTTLQDCLGAKNVPVSFTNSAG
FSALAQPYNLRLAYTPAVIVLPTTTQHIIDAVLCAGKSNVKVQAKGGGHSYASFSSG
GQNGAMVVDLESFQQITVGAGGVAQIGGGVRLGDMALGIFNQSQRALPHGTCPGVG
VGGHASHGGFGLDSRLWGLTLDTIVGLDVVLANGSFIHATSTAYPDIYYALRGAADS
FGIITTFYLQTLPAPASVVNWQYSIPGMFASAATSTAVFAHIQTFAQNASVVDRKLGL
GIYLDGSGFSVSGTYIGDLATFNSKIAPELLRGLPTPSSSSVKAVDWIQSLTLLANGQP
LQQPTSRTSYTPHDDFFAKSLVVPSSAPLTTAALTSYFTYIIQNGVNAPNPWYSIINLY
GGPDSQINVPSPSSSAYSDRSALWVLQHYGYTGNTGSPYPPASLTFVNGLNTAITSAM
PSTTFGGYLNYVDPSLTAAQAHTLYYGTTTYAKLLSIKNVVDPGKVFSNPQSIGN
(SEQ ID NO: 45).
>BBE12.1
SKINPINPFFENDIGCEKVKYPSEILAPIPYLKECLTTIDGLVVYPDDPY
YYHSIKVMNCRNSYFPVVVIYVTTISDIQKSIDCANTLNISVTARSGGHSYEEYGNGG
KSGAMVIDVQELNQIDINFETKTAVIGTGNRLGTIYYKLYKDGFLLPAGYCLNVGIGG
HATGGGYGMVGRKYGMASDNIVSAEMVTANGTFLSPINSKHHSDLFFAIRGAGNAG
YGIITSLTFKIYPIWPIVTTINIAYFSDQIELAFNILAKVAKNLSENLNAYIFLIPKINNNS
NTLLINATYLGSEEEARNAAKELIELSNPAYIKFKEMPWWKAVKAQSPPFNRQPFKV
TSYTIPPQGFSREAIRFLVNFINNMECMSGVLFDLYASDALSRISVNSSAFIHRNILFVM
ELETMLDGHSKNVQNKCVKKHLQFSQEFQAKYTSYFSYQDYIDKDLPDWLTRYYGS
HVPRLIETKKKYDPDNLFNWKQSIPTEL (SEQ ID NO: 46).
>BBE13.1
SPFYGPPDALTSCLATASVPYAAKNSSQWIQDTKPYNLRLAYVPEAV
AIPTTIKHIQDAVKCARQHGVRVSAKSGGHSYGSFGFGGEDGHLVVVMDAMDKVTL
NKDMSCTIEAGARLGHVANELFNNAYRRALPHGTCPGVGITGHALHGGYGMSSRTY
GLTLDRLVAATVVMADGSIKHSSVWDTPNFHWALRGAGSSFGIVAELDFMTFAAPE
VLTSFSIDLDWTEEEAVEGILAFQEFGVNAPKELNMQIFMGPRGQTIQGLYHGSLEGL
NTALRPLLGEINAQVSKTKTMNWIESVGHFSDGQSIVQRRPYDRHSTFYTTSLMTHA
LTRHQVESLANALFTNARDPSARKSWYLLLDLFSGPNSAISEKAPSDTAFPHRDKLLL
YQFSDGGTNGVYPPEGFDLLRRFRESVTSSMVDGEWGMYANYLDTQLDGDTAARL
YYGGNLERLRVLKGEFDPDDVFWNPQGIRPA (SEQ ID NO: 47).
>BBE14.1
YVSLTEAPPTSLIGCLQSIKGNKKSYLSIPSSPTYNNDRLGYNRNFVHK
PSAIFHPASEADAVAAVVCAAAHNVSIAPRSGGHSYEGYSGGGKTASLVIDLNLFLQF
AIDHDTGIATVGAGNRLGTLCTKLWKAGEYLIPAGSCPGVGIGGHALGGGLGIAGRK
YGMLSDNIVGLTMVAANGNILEVNARSRPDLFWALRGAGGGSFGLVTEFRIQAYKA
PARVTTMEVKYPWSVHQNVINAFGIWSKSVVDDFTGMLYIGQGKVRVLGTFLGSQE
EAQTALKSLFTLTGLPQQTTFNESTWYQAAARWAEMEGANLEDPYTEHALAFRFRS
LLYRQPISDSELAIISKHINNPPNNPDGQVTISVSLEIWGGKIDYPSSASAFDNHRGILYS
IRYGVEWNVSSPDSDSICTECMEWNDHFAKELQAAYSSGQVLEAYQNYIERDIPNGL
QAYYGDNLSRLIEIKKSVDPGNIFVFPQAIPLS (SEQ ID NO: 48).
>BBE15.1
QYTSLREQLASNGIRAVFADDVEYGNATRGYNMRYTPSPIAVTFPTN
TSQVSLIVKAGAKEGMRVVAKSGGHSYIANSLGSDSDPPALVVDLGAFKNITYDGET
QTAVIEPGNRLGEVALALNEYGRAIPHGRCAWVGFGGHSGFGGWGFSSRMWGLTL
DNVLSATVVLANGTVVTASEDENTELFWAIRGSSSSFGIVTALTMRTHPVPPSGTFFE
YIFDLDIATASKAFSDFQKYALSPSLPNTFGGELFVLKGEEKGRVYIVFLGSFYGDTER
YAQVVTPFLDTLPPWRNDSIVLNGTWIDVLAAAAVGNLTVQGEMPRDTFYVKSLMT
PQDVLLTEEAMHGMMRYLGEEGFDSDAFWHVEIEIYGGVQSAINSVPLNTTAFGHR
DTLFTFQPYVSTADFLPPWDDAIFGFVDGIMSSITDNMPSDWEYGAYANYIDPRLPD
WQHRYYGANYERLQKLKGEVDGDDVFKFITSIEVPEEGKR (SEQ ID NO: 49).
>BBE16.1
RNDVTETDIPAFAAATITADDPRYPSLVLGTNHRFPGRPDYVRVVTTT
AQVAAAVTEAVAAGRRISVRSGGHGFEDLATNGVEVLLDMSEMTEVRYDEERRAF
AIEAGATLGHVYRVLFKGWGVTIPAGECPEVGVGGHIVGGGYGPLSRSLGAVVDYL
YAVEVVVVGEDGVARAVVATREDGDPHRDLWWAHTGGGGGTFGVVTRYWMRAP
GADSGSDDPAELLPKAPAAWRSGMAVWSWETMTEESFAALQANFGAWFERNSDPD
SPYAALAGFFHGTHRSGFGLTVGAHMDDATADAERLMTSFFEEITAGVDARPLFVD
QQVRPWLYMSSYPGWGDPGDPFTRRIKIKAAYLRKGYSATQTAALYRHLTAGEASP
VQLILIGYGGRVNAVAPDATAAAQRDSVLKAAYMAVWGAEADDETNIRRVREMYR
EVYAETGGVPVPNDANDGSYVNYADTDLADPEWNTSGVPWHTLYFKDNYPRLQRV
KKRYDPRNVFRHALSVGLPG (SEQ ID NO: 50).
>BBE17.1
ADEASCPPAPGPAMVGRGDPRYRSLASRGYNRRFVGGPEHVWVVGT
TAHVVRAVQQAVDSGRRITVRSGGHGFENFVADPAVEVVVDMSAMTAVSYDPDRR
AFAVEAGALLGEVYRRLYLGWGVTIPAGWCADVGIGGHVLGGGFGPLSRLMGLSV
DHLYAVEVVVVDRAGKARRVVATREPSDPNRDLWWAHTGGGGGSFGIVTRYWFRT
PGADGDDPSALLPAPPSGVLSFSVTWDWQKLDRTSFARLVRNHCAWAERHSAPHSP
YLALYSELALTRRPSGTVLMIGQVAADSGAERMLDEHLAAINRGVPVQPVRTVRNH
SWLAAALAGSPGDPGPVYRLKVKSGYLRRRFTDRQIDTLHHHLTRSDYDHPGGSVSL
YTHGGKVNTVAGDATATSHRDASIKMFYVNGWEDPRDDARHIAWLRELYRDLYAD
TGGVPAAADGAFINYPDTDLADPAWNTGAPWHTLYFGENYRRLQRIKAKWDPRDV
FHHALSVRPALRRG (SEQ ID NO: 51).
>BBE18.1
RPPYATGDSPVPAAEVLPGDPRYPALVTGFNQRFTGTPAAVVVVTTQ
DQCVAAVRAAVAAGRRVTVRAGGHCYEGFVTDNPGGVVLDVSGMRAVTRTPDGGI
RLESGCTNWDVYELLYKSHGVTLPAGSCYSVGLGGHVVGGGYGLMSREHGLTVDH
LSALDVVVVDADRSVRVVTARRGDPLTGDLFWAHTGGGGGTFGVVVAYTFRDLPA
PPPTVQLATTTWQWSALDAASFATLLRNYGGWLAANSGPDSPGRRLFGLLKLSHVS
AGTVTLLTQASGEDPALLDGFLGALDDGMPAAALPTTTRRTMPWLQATQTLNGSGP
NQRGKYKSAYMRRPFPDRHVAALHAALTDASYRNPQALVQVDTYGGRINAVASGD
TAVSQRSSIMKLQYQTYWTDPAEDELHLGWIRRCYASVYADTGGEPARDDVTDGCY
VNYPDVDLRDWPTLYFGPNYRRLQEVKGRWDPTDQFHHAQSVRPPGR (SEQ ID NO:
52).
>BBE19.1
HSHEDFLQCLSHHFSNSTTFAKLIYTPNDPSYISVLNSTIRNPRESSLST
PKPLLIITPSNSSHVQASVYCSKKHGLQIRTRSGGHDYEGLSYVSEVPFVIIDLRNLNSV
IVDVHEKTAWVEAGATIGEVYYRIAEKSGNLSFPAGVCHTVGVGGHFSGGGYGFLM
RKYGLAADHIIDAQLVNAEGKFLDRESMGEDLFWAIRGGGAASFGIVLAWKIRLVPV
PSTVTISIVDRNLEQNETLKLVNRWQSVADKFDEDLLITVGFQTVNSTDREGNNKTII
QATFVSLFLGKVDGLLSLMEKSFPELGLKRGDCIELSWIEVVHYFAGFPLRGELEDLL
NRTPQEGLSPFKTKLDYVTKPISEKVLETVLQRLYEEEVGKGLIQFFPYGGKMSEIPES
EIPFPHRAGNLYKIFYRIEWDEGERDVKAVERHLDWVRSVYNYMTPYVSKNPRGAY
LNYRDLDLGTNNDKGPTSYAQASIWGKKYFGSNFKRLVHVKTKVDPKNFFRNEQSIP
PVPSPGVNKNNNYTP (SEQ ID NO: 53).
>BBE20.1
HSHEDFLQCLSHNFSNSTTLAKLIYTPNNPSYISILNSTIQNPRITSPSSL
KPLLIITPSNASHVQASVYCSRKHGLQIRTRSGGHDLEGLSYVSEVPFVIIDLRNLISIN
VDVHEKTAWVEAGATIGEVYYRIAEKSGNLGFPAGVCHTVGVGGHFSGGGYGNLM
RKYGLAADNIIDAQLVNADGELLDRESMGEDLFWAIRGGGAASFGIVLAWKIHLVPV
PSTVTMFTVDRNLEQNETLKIVNRWQYIADKFDEDLLIFVRFLTVNSTDQEGNNKTTI
QASFFSLFLGGVDNLLSLMAKRFPELGLKREDCTEMSWIESVHYSAGLPSGKLEGLL
NRTPLEVPILPFKVKLDYVKKPIPEKVLETMLDRLYEEEVGKGLIQLFPYGGKMSEIPE
SEIPFSHRAGNLYKILYYVKWDEGENDSKAVERHLNWVRSVYNYMTPYVSKNPRGA
YLNYRDLDLGTNNDKGPISYAQASIWGKKYFGSNFKRLVHVKTKVDPKNFFRNEQSI
PPLPLPGAKKNNNNGP (SEQ ID NO: 54).
>BBE21.1
LSDTTPEKFYQCICKNSDFSDPFSKAFFTPNTDSFNTVLNSTAQNLRCL
MPTVPKPVLIFTPMAESHVQAAVICAKQLNLQLRVRSGGHDYEGLSYISVMDSPFVIV
DLSKLREIDVNIEENSAWAQAGATVGEVYYRISEKSKTHGFPAGLCTSLGIGGHITGG
AYGSMMRKFGLGADNVIDARIVNANGTILDRLSMGEDLFWAIRGGGGASFGIILSWK
LKLVTVPSVVTVFTVPKTLEDGATKILYKWQQVADKIDDDLFMRVVINVVDKKDKK
GERTVQTAYNSLFLGNVDRLLQIMNESFPELGLTQKDCTEMSWIESILYIAGFPTKTPP
EILLQGKPTFKNYFKAKSDFIKEPIPETGLDGIWKRLLEEDSPLMIWNPYGGMMANIS
ESETPFPHRKGVIFKIQYLTLWTEPDQELATKHVDWIRKLYSYMTSYASMFPREAYV
NYRDLDLGMNKNGNSNSSFVQATVWGNKYFNNNFNRLVEIKTKVDPENFFKFEQSI
PTLPLTKKRKGKKINH (SEQ ID NO: 55).
>BBE22.1
AVTKPGIGEFLGCLRSWPSPESPITDDIFTADNTTTFLSSYLAYTKNTR
FESPNYQTLLAIVTAKNLSHIQATVVCAKSNGVQIRIRSGGHDYEGLSYISSVPFVILD
LFNLRSITVDVPSKQAWVEAGATLGELYTKISDASETLAFPAGVCSTVGAGGHISGGG
YGNLMRKYGLTVDNVVDALIVDVNGKLLNRSTMGEDLFWAIRGGGGGSFGVILSW
KINLVDVPEILTVFRVNKTLEQGGIDVLYKWQLVSSKLLDGLFLRALSQVVNRTITVE
FYAQFLGRADELVAIVNQSLPELGLKRQDCLEMSWLNTTLFWEDLPVGTPTSVLLDR
PSKPEKFFKSKSDYVKKPIPKEGIKKLWEALLEFNNNNIVYMQWNPYGGVMDTVPA
AATPFPHRKGNLFEVQYYTSWLDANATMGSLDMMKKLYEVAEPYVSSNPREAFLN
YRDIDIGSNPSNETNVEEAEIYGSKYFVGNLKRLMEVKAKYDPENFFKNEQSIPPARV
KQ (SEQ ID NO: 56).
>BBE23.1
AVESFLQCFTSDIKNNSSKIIITKSSSAYFSAVQSSIQNLRFVTSATSRPE
AIITPLHDSHVQAAVICSKKKRILIRIRNGGHDYEGQSYVARAPFVIIDLFNLRSIDVDIE
NESAWVESGATLGELYYRIAEKGKAYGFPAGSCPTVGVGGHISGGGFGPLFRKYGLA
ADNVLDAKIVDVNGRILDRKSMGEELFWAIRGGGGSSFGVILAWKLRLVPVPPSVTV
FKISRTTEQGATKLLSKWQNIADKLHEDLFLHSVIGVGNKAGTNGDKTIIIEFSSLFLG
PVEKLLPLMQDNFPELSVDRSDCTEMSWIESVLYFASISRNESEALLSRTQQSKSFFKA
KSDYVNEPISEAGLEGLWQTLIEVKAYLILTPYGGKMSEISDSETPFPHRSGNLFKILY
MVTWDDYRENEIGESLMSKLYDYMAPYVSKSPRAAYLNYKDLDLGRNNDVHTSYA
QASIWGLKYFKNNFRRLVHVKTLVDPGNFFRDEQSIPSVRSFRKK (SEQ ID NO: 57).
>BBE24.1
EHCLPGDGTGCWPSQAEVKALVDSLSSEAEVYLPTGPNADMYMSYA
TDTPGNTLKPSSPGLIVFPMNEGDVRKVVTFAAKFRIRLVGKCSGHDQLQRSNGYGV
IQLVTTKGFRNISYNETDHTITFGAGNAHKDVYAFLADRKRFAVGGTWAYVCPAGC
LSNGCYGFFTKEFGSGADNVVGLRFMLFNGTTLNLDAAGPHRDLFWAILGGGASSY
GVLLGITAKTYEAPDQFIIMKVFLSIWPTGLTKDQAPSVFTRFLTNLTWYQALPPHIS
WQLDNNPAEQRVLLQISYTGRTTVDVATEELSGLVAEWTEAGYFQEHGVEGYSDVA
DFAVKNSGAGPPIRRVSQSIYVDANQDALDKVAALWETLYSDAYMSPVGSDPFDGA
FNFWAQYRYGPGSYAGKAPHVARGMRTAMMEIAHENVWMRQADDSFRAAISNDV
KELMEPLGNTSYQNHCSTWAEYNVYVDYKTRFYQNWEGLTAVKKKYDPCNLYVV
QYGPGWDLPKATCGLSTTSTKRPPPKRPPPRRHA (SEQ ID NO: 58).
>BBE25.1
NPQENFLKCFSQYIPTNVTNAKLVYTQHDQFYMSILNSTIQNLRFTSD
TTPKPLVIITPLNVSHIQGTILCSKKVGLQIRTRSGGHDAEGMSYISQVPFVIVDLRNM
HSVKIDVHSQTAWVEAGATLGEVYYWINENNENLSFPAGYCPTVGAGGHFSGGGYG
ALMRNYGLAADNIIDAHLVNVDGKVLDRKSMGEDLFWAIRGGGGENFGIIAAWKIR
LVAVPSMSTIFSVKKNMEIHELVKLVNKWQNIAYMYEKELLLFTHFITRNITDNQGK
NKTTIHSYFSSIFHGGVDSLVDLMNKSFPELGIKKTDCKQLSWIDTIIFYSGVVNYNTT
YFKKEILLDRSGGRKAAFSIKLDYVKKPIPETAMVTILEKLYEEDVGVGMFVFYPYGG
IMDEISESAIPFPHRAGIMYEIWYIASWEKQEDNEKHINWIRNVYNFTTPYVSQNPRM
AYLNYRDLDLGKTNFESPNNYTQARIWGEKYFGKNFNRLVKVKTKVDPDNFFRNEQ
SIPPLPLRHH (SEQ ID NO: 59).
>BBE25.2
NPQENFLKCLSQYIPTNVTNAKLVYTQHDQFYMSILNSTVQNLRFTS
DTTPKPLVITTPLNVSHIQGTILCSKKVGLQIRTRSGGHDAEGMSYISQVPFVIVDLRN
MHSVKIDVHSQTAWVESGATLGEVYYWINENNENLSFPAGYCPTVGTGGHFSGGGY
GALMRNYGLAADNIIDAHLVNVDGKVLDRKSMGEDLFWAIRGGGGENFGIIAAWKI
RLVAVPSMSTIFSVKKNMEIHELVKLVNKWQNIAYMYEKELLLFTHFITRNITDNQG
KNKTTIHSYFSSIFHGGVDSLVDLMNKSFPELGIKKTDCKQLSWIDTIIFYSGVVNYNT
TNFKKEILLDRSGGRKAAFSIKLDYVKKPIPETAMVTILEKLYEEDVGVGMFVFYPYG
GIMDEISESAIPFPHRAGITYEIWYIASWEKQEDNEKHINWIRNVYNFTTPYVSQNPRM
AYLNYRDLDLGKTNFESPNNYTQARIWGEKYFGKNFNRLVKVKTKVDPDNFFRNEQ
SIPPLPLRHH (SEQ ID NO: 60).
>BBE25.3
NPQENFLKCLSQYIPTNVTNAKLVYTQHDQFYMSILNSTVQNLRFTS
DTTPKPLVITTPLNVSHIQGTILCSKKVGLQIRTRSGGHDAEGMSYISQVPFVIVDLRN
MHSVKIDVHSQTAWVESGATLGEVYYWINENNENLSFPAGYCPTVGTGGHFSGGGY
GALMRNYGLAADNDAHLVNVDGKVLDRKSMGEDLFWAIRGGGGENFGIIAAWKIR
LVAVPSMSTIFSVKKNMEIHELVKLVNKWQNIAYMYEKELLLFTHFITRNITDNQGK
NKTTIHSYFSSIFHGGVDSLVDLMNKSFPELGIKKTDCKQLSWIDTIIFYSGWNYNTTN
FKKEILLDRSGGRKAAFSIKLDYVKKPIPETAMVTILEKLYEEDVGVGMFVFYPYGGI
MDEISESAIPFPHRAGITYEIWYIASWEKQEDNEKHINWIRNVYNFTTPYVSQNPRMA
YLNYRDLDLGKTNFESPNNYTQARIWGEKYFGKNFNRLVKVKTKVDPDNFFRNEQS
IPPLPLRHH (SEQ ID NO: 61).
>BBE25.4
NPQENFLKCFSQYIPTNVTNAKLVYTQHDQFYMSILNSTIQNLRFTSE
TTPKPLVIITPLNVSHIQGTILCSKKVGLQIRTRSGGHDAEGMSYISEVPFVIVDLRNMH
SVKIDVHSQTAWVEAGATLGEVYYWINENNENLSFPAGYCPTVGAGGHFSGGGYGA
LMRNYGLAADNIIDAHLVNVDGKVLDRKSMGEDLFWAIRGGGGENFGIIAAWKIRL
VAVPSMSTIFSVKKNMEIHELVKLVNKWQNIAYMYEKELLLFTHFITRNITDNQGKN
KTTIHSYFSSIFHGGVDSLVDLMNKSFAELGIKKTDCKQLSWIDTIIFYSGVVNYNTTN
FKKEILLDRSGGRKAAFSIKLDYVKKPIPETAMVTILEKLYEEDVGVGMFVFYPYGGI
MDEISESAIPFPHRAGIMYEIWYIASWEKQEDNEKHINWIRNVYNFTTPYVSQNPRMA
YLNYRDLDLGKTNFESPNNYTQARIWGEKYFGKNFNRLVKVKTKVDPDNFFRNEQS
IPPLPLRHH (SEQ ID NO: 62).
>BBE25.5
NPQENFLKCFSQYIPTNVTNAKLVYTQHDQFYMSILNSTIQNLRFTSE
TTPKPLVIITPLNVSHIQGTILCSKKVGLQIRTRSGGHDAEGMSYISQVPFVIVDLRNM
HSVKIDVHSQTAWVEAGATLGEVYYWINENNENLSFPAGYCPTVGAGGHFSGGGYG
ALMRNYGLAADNIIDAHLVNVDGKVLDRKSMGEDLFWAIRGGGGENFGIIAAWKIR
LVAVPSMSTIFSVKKNMEIHELVKLVNKWQNIAYMYEKELLLFTHFITRNITDNQGK
NKTTIHSYFSSIFHGGVDSLVDLMNKSFPELGIKKTDCKQLSWIDTIIFYSGVVNYNTT
NFKKEILLDRSGGRKAAFSIKLDYVKKPIPETAMVTILEKLYEEDVGVGMFVFYPYGG
IMDEISESAIPFPHRAGIMYEIWYIASWEKQEDNEKHINWIRNVYNFTTPYVSQNPRM
AYLNYRDLDLGKTNFESPNNYTQARIWGEKYFGKNFNRLVKVKTKVDPDNFFRNEQ
SIPPLPLRHH (SEQ ID NO: 63).
>BBE26.1
GFPAANLSSVLSSPLLGLSPATEIFYPSDADWTNETQRWTLHDSPTYL
AAVKPATERDAQKLVAFAAKSNISFLATGGAHGFTTSYGTLQNGIMLDLSNFRNVSV
DAAANTMRIGAAVRFGDVYEPLYNAGKEITTGTSSCVGMLGATLGGGLGRYNGIHG
MILDALISVRLIIATGEIITASTTENADLFWAVRGAGFNYGIVIEATYRITDLTSQYVIN
ADFAFPMNASTALLTYLKSFETDMPEKLSLITYVTYVAAYGGLSFTFNAVYAGPKDE
LDQLLSPLLRDATPIRENITVIPWKDLTYTAFFAAEPSMAACTKGLYRNVYGGSVKA
YDISSFNTFFTEMQDFYLEYPDAQSSVFFIEHFPKAKALSVPDNATAYPYRDITAHLL
WNFGYPAGDLEKPVNAFGVKARADLQASSGFLPQQIYVNYAHGDEAPEILYSARNL
PRLRNLKEVWDPENVFRFNEPVV (SEQ ID NO: 64).
>BBE27.1
LSPSFEHVVDRGSNIFSELDAQLSPNASIILPADPLFQTAGARWQAYSR
PTYSAVVEVAVEEDVQKTVRFANKYSIPFLAVDGAHGFISTLGRMQNGIAISFSKMK
GITVSRNGKFADLNPGLTNGDIVRTLWAQNKYTVTGNCMCTGIMGIMLGGGHGYLQ
GLYGLAADQILEARVVLADGSCVTASPESNPDLFWALRGAGHNFGIVTGLKYKVYE
SLSLWTEIQMIFKGDKVEEFFEVANNLTQEENHPAKLIHWSNHIRRPDIDPDNAILNTY
FIYQGTPTELEKYVAPFRALGPVGETRTTAISYPDLFPLNRANETNLETCGKGLYRHL
FPVGLKRYNPAAQRKIYNLFNNLTATYPSIAFTSTFVMEGYSTQAVTSVPDESTAFPF
REDNILQSPVWGYTDPSLSAEIIAVSKEMRRILVDASGSNKLHAYVNYAFGDETLPEM
YGHEPWRVKKLKQLKKKYDPNGRFNFYAPII (SEQ ID NO: 65).
>BBE28.1
LHPDPGNIRSPAAVDNDGLPISLQSCLGATGAEVVYPTDAEYANLTLP
DNSNYHPHPQAIVIPKSTQEVAAIVRCVSAEQGRVTLSPRGGGHGYAAYGFSGQVVI
DSSQMTDIVLNEPAQEVTVQMGQTIGILALTIGRAGYALPHGTCPGVGIAGHSLGGG
WGYTSRQWGWLVDHLVSLELVDVAGRIHTLGPTTNTSDSDPDLWWALRGAGSNNF
GIVTSFTYRMERAPTAITNYNIDFATHSDCVQALLTLQDLGSQPDGLPTSLGGELVITG
GYTPPNAYCSFTGQYFGDPSSYNTTIQRFLAPLAQQSIQPQSTTSASFTNWVDALTDL
MGPLDDPSIPQPYYAQSLLDPGTPNYTPTTASSILTAVQPAGPAAHISFDLNGPSSATN
QPPQNDVGPMSFFHRKSLFLAQIYAADFPGFDDPGRKEALDTIWAISDAVRSADPEG
KWHAYQNYIDPDLDNWGEMYYGANLERLKGVKERVDPNHVFDFPQGLGRA (SEQ
ID NO: 66).
>BBE29.1
TRTPLNSCSPTDKRSNTSTETHASFRDCLLSELGRLNSFSSKTVLPGDI
SYPLSTLTYNLAIQHNSSAIVYVSTAKDVSKAIQCASNFRVPVVSRSGGHSYASFSSSP
NGLIIDVTNLKDFSFETNGVGKVGELVTFGAGLRLGDLDMALQPHERAIPHGVCPYV
GVAGHATCGGFGTASRMWGLFSDHVVEMEVVIANGTILKASEKINPDLFFVRIPPSY
VHAMRGAGPSFGIITSLTLRTRQAPRNVILFEINYRFPNPDVAAATLNHFQSWGLKSA
PSQLNIRWSVKLNHGPNSPKGVDLDWKLQGTFLGHMARFNRTINKLTKDFRVKGEL
VKLERLDWVESIWALGGDRTLSSEGINPSTYNASFYAKSFIVRDVSPLNYYEWKTFTE
KLYGIASGIQKSKRLDWFLEIDLIGGRYQGQETGVRGLHSTTTSFGPRDALLLFQMGG
NGPKGAMLYHGGLMESIRVNSEEIYSSLGGGQKELTGENCYVDSEFSADRAHREYFG
QKNTAILKNLKKVWDPSRVFNHPHSF (SEQ ID NO: 67).
>BBE30.1
QTGKTSPNVFEPDNFDINAALYNLGVDVSAIPALSSLQSRSTESACSA
ACSALGFLYGPSRAFAQNTTAYANATGSYWSVQQEEVRPYCIFKPSVNTDVSILVLLS
RYTGCPFAIRSGGHAAFAGASSIQGGITVSLKDMSAITLNEDRSVVSVGPGNVWVQV
YAALEPYGLAAIGGRVSSIGVGGLTTGGGISYYSNLYGWACDNVESFEVITASGAIVT
ASAHSYPDLYWALRGGGNNFGIVTKFNLYTIPSPEMRGGTRAFAEDQFSKVISAFISV
VNDAPNDGNAQHWVAFIHTQGQNLAAVELTYVKNVTEPAIFAPYRAIPAIEDTTASK
TLVEYCDTVQEVNPDGLREMYWTLTVRLDEAFANWVTGYFYSVLPQVWSIPGINPG
LVNQGITIPMLKNMTRNGGNALGLDASEGPLHLLLMSIWWENADDDETIVAWAKNF
WDTVSAKAKSDGVFHEYVYMNYASQYQDVVAGYGAANKAKLQSIAAKYDPKRVY
QRLQPGYFKLNGAPASDAS (SEQ ID NO: 68).
>BBE31.1
APGSNFKALQQCVNGALAGGGNVEKRVQSPANATWDDAYIGAIIKP
ETPAMIAFAESFTEVGALMGCANTTGYQAVPRSGRHHFEGWSALNGSLVIDVSSLDY
IKVSSDLSNATIGAGANLGQIYTELSVVNKTFLGGICPTVALGGYLGVGGYSLQHRAL
GIAVDQVLSFKAILASGELVTVSPTSYPDLWFAARGGGQYAFIVEATVKILTLPRSAM
VVGFYNNSDTRYEVARKWLDWAPTAPKELTTQLNVYSNRTHLIGWYLGGSPEQLRT
ALNSSGLLDVADGVVSIDGNCSTANSRMYWQDPSTRCTDDASAYKAFLKVYNTESI
NLTPIQPAFRLDNTPALPSEPVAEPWPRFGVISKTYFTQKSRPITNDTLKELINSSASLG
EEAGFWGEWTSFGIANPTTTSAFPWLKEAQALLRIEMNSPKNVTQYNQNRAWLLNF
EKFFRPRVGNASYAGYVDADISINPLTAFYGNNTCRLISIKQKYDPNNFFRNPFSVPTQ
VPKGISC (SEQ ID NO: 69).
>BBE32.1
QACNNHQLTDAILQEFSQTLSGDLVLPTDGLYQWARLIHHTNFDGIY
PVGIVFCETPEDVSKAILFARQFGLHVTARGGGHSYEGYSVTGGLLIDVSRMNSVSVN
PAAMTAVVGAGAVLIDVYHGLYYPHKLSIPGGSCPSVGIAGYLLGGGVGLQSRTYG
VGCDRVLEIGVVLASGEYVVASPTNHSDLYWAYRGGGGGNFGVVTHFKMQCHPVD
RLSYAIITWDWEAAAPAFNAWQNWLKGLGEDYRNFSIFKFLVNAGGDGGLGPKVNL
IVQFDSQSNTGTGELETLMAPLLNTAHEHIVSQTIMNGDFFEVTMEIMAGCAWPKTD
LDTEFLRCHTVGNPAFPMASLPRDTYKAKSTFFADVISEKGIETCIKAIEDRFNSNLPD
NSSQFTCALQFDSEGGIMGDVPKDATAFMHRDCLMHCQYLAYWPAEGDAWFDDNP
DYSYCDVSNGSMQWISDAFNVLWPFGNGHAYQNYIDKEQPNWLYAYYGENVERLR
TVKAKYDPDNIWKFEQSIPPA (SEQ ID NO: 70).
>BBE33.1
CSGTAPPAAAPQAPPSGPPGPSVPSGPPDWAALRSRLSGELLLPDQEG
FTYQGFNPVWNQRTPAAVARCASVDDVRACVEVARSRVPIAARGGGHSYAGYSTPE
NGLQVDLRRLATVEVLPGDQVRVGAGAQLGEVAQVLAASGRCLPTGTCPTVGVAG
LTLGGGIGVLARKYGLTCDHLVSATAVTADGRVVTASAEQEPDLFWALRGGGGGNF
AIVTELVFATVPAPQVTVFSLQYNAGVADVLGAWQDWIRQAPNELWSNVVVRGGP
EPTCRVGGCFVGPVGGLTSLLSALPAPASRFVQPQSYPEATQYFAGTGIQHESFVASS
RIVRSPLTDPARAVALMNGQSAESYLIFDGLGGAVSDVAANATAFPHRGALACAQIY
FKTPPAGATETAVAMGEVRDRLGELVGDSSYVNYIDPQLPRWAEAYYGDNLARLRD
VARKYDPDRVFAFAQSIPR (SEQ ID NO: 71).
>BBE34.1
CSPVLPNEIPAGVRWEDLRRSLSGALVVRGDAAMDESGRAFNPLFDV
NHPGAVAFCASDQDVARCVEFATSARLPIAARGGGHSYAAYCIPNDGLVVDLARMA
AVSVTGTRAVVGAGARLIDVYAGIAGAGRMLAGGSCPTVGIAGLTLGGGVGVLTRR
FGLTCDQLISARVVTADGKIRVVSADTESDLFWAIRGVGGGNFCIATELAFETAASTD
LTVFTLDYAAGEMAPIVHRWLTFMAGAPDELWTTLHAIGGAIPQCRIVGCVAQGVN
SQDVIESLRGGIGVRAADSFIAEMTFLDAMKFMGGCTTLTVAQCHPSWTGPGSGQLK
REAFVASSRMVPHPDVDTARIETLLAGKPGLTFIFDSLGGAVRRISPDATAFPHRQAA
ACIQIYHGVGADPAVAHERVSQARDGLGDICGPAAYVNYIDPGMPDWATAYYGDN
LPRLRGIAAAYDPKGIFRFAQAVRP (SEQ ID NO: 72).
>BBE35.1
GGSAGSKIASSSVVEVPPAPTVVSKLSAEQWQGLSEQLNGELILPTNV
VDYSQERLVFHTRFDHIYPQAVAKCRTTDDVIAVLSFVKQHDLHIVPRSGRHGYAGY
STTEGVVLDVSQLNSISIDDGYATIGAGARLVNIYDQLTNQSVCIPAGTCLSVGIAGLT
LGGGFSVIGRAHGLTCDVLESAQIVTAQGELLTCNNNENSDLFWALRGGGGGNFGIV
TSFVFRTHKTQDINVFEASFSFNDFEQLMSKWQVLAQSWPDEMWAQCLPNWINGSV
RVTVRAFCLNQADEARSYWEQFIAAVHTSPLSNNESTESYRDIMLGNCVNNIPSCHLS
VQSAGGRMQRSAFGASSDFFEGIIPPSGIKTLKGFIEKSINEGKYGMIIFNLLGGAIKHT
EVTETAYPHRRANISAEYYAPINISSTNEQIDETQKWQNQFREIMAPWSTGGAYVNY
MDPLILDWQQAYYAGNYYQLQKIKSKYDPEEMFNFTQSVELA (SEQ ID NO: 73).
>BBE36.1
CATSRPAPQPEAAPLCRCAPGQPCWPTRDDWQKFGASLRGKLEQPQS
PFAPCRSDAAGEACATAIRNSTNPFYLQDQPGGTESTGWLGAWTAASSAYAVVAKD
ASDVVAAVKFARRSRLRLVIKGTGHDYLGRSNAPDSLLVWTHQMRKVDVRDAFIPQ
GCGAAEPAPAVTLEAGTRWLEAYQEVTVKHGRYVQGGGCTTVGAAGGFMQGGGF
GSWSKKYGIAAASMLEAEVVTADGRVLVANGCQNQDLFWALRGGGGGTFGVVTR
VTLRTHPLPDYFGAIDGSIVAKTDAAFKDLLEHFLLFYREELSNEHWGEQVKVRRSN
SLDLSLVFEGMSAKQAGEVWRPFREWVEANRDTLTMKAELVEMPGNRMWDAAYL
KNIPGAIETDPRPDQPPDRFWWWAGDGGQVSAYWYAYQSRWVPLDLFEPANASGF
AATLFDASRHWSVALHENKGQAGASAEARERGREISMNPAVFKAAALIIIAAAANAY
PGVPGHEPDMTEGESAKARVGAAMKVIRAATPGAGSYLNETDYFESDWQREFWGE
NYARLSEIKRKYDPDGLFFCHHCVGSEGWSEDGSCRSTR (SEQ ID NO: 74).
>BBE37.1
VSDKNLKPNCRCLPSEPCWPDQQQWDTLAKSLKGKLIKPRLPLSVCE
KNSDSKECKLVLQNIKNPFYMQSDSGRNESQGWYGAWHNQSSSYAVEAEDTQDIVK
AANFARKHNLRLVIKGAGHDYLGRSSSPDALLIWTHNMRDIEYNKHFHPQNCPKNK
EYSAVTVGAGTRWLEAYNVVTNHHHQYVQGGGCTTVGAAGGFPQGGGFGSWSKQ
YGTGAGGIIQAEVVTADGKVVIANECQNQVLFWAIKGGGGGTYGVVTNLTLRTHKL
PSHFGLISGEITTDSDKAYKKLIKEFLLFYSSKLNNEHWGEQFAFRPNDKMTIAMVTQ
GLAEKEALDIWQPFKKWLESQPDLHYKLKYIDIPPTQIWNYDFWHKNYPDMVIKNIG
PDARDGEFWWASNTAEVSAYWYTYQSWYLPEKLFDSKNIDKTVDTFYKVSQLAPV
SIQINKGLAGASKEAIELTKQTSMHPGVYDAGALAIMSYSNDKPEFDKPKMTQEIEQK
VDNIYKAMNMIMALAPDAGTYANEADYFQKNWQQVFWGSNYSKLLKIKNKYDPN
GLFYCHHCVGSEYWQQDGMCRK (SEQ ID NO: 75).
>BBE38.1
ATSQWNILRSRLQGTLVLPTDSGFAAAKQLQLGQYDAVNPQAVAYC
QNADDVRTCVLFSQYTGTPLRVRSGGHNLAGWSTGDNALIVDLSRINQATVTGSTV
QLGPGTQSIDALSQLKPYNKQVIAGTCPTVCAGGFLSGGGLGWQTRKFGIGADHVVS
ARVATSDGRITYCSPFVEPDLYWAVRGGGGGNFGIVVNFEVTPIDAPVLTAYETVWS
FDNAATTIAAWEHWTANAPRELGSSLIVVPPNGYYGYPSSNPLVRIYGGYHGVQAD
ADAQLDQLALAVGAKPLSRTSQQFGFADGMQYLYGCGDITELACHRAGSNPDAVLP
RTPNQRQSYRLIDRQLTTADATDVLNAWSAHPELPYRFLQSFALGGAANDVPSGATA
YVHRSAQSLLGYQVGFQDSTDVPAADDWAAAGAAALAPRAGGSYINFPSSVMNGD
WAADNYGDNYARLVQVKYAYDPTDLFQHPQSVGS (SEQ ID NO: 76).
>BBE39.1
RTTFRRRRPSDASWPSPSAWKQLNDAVDGNLSPVSSPLSVLKADPAG
AAKRVWQNLKNPYYIGDEPGLTQTLGLVDAWTTAPSVYAVAARNAHDIAAAVNFA
RDNDLRLVVKGGGHSYQGTSNAPDSLLVWTRHMREAAMHAAFVPQGCEGTVPAQP
AVTFGAGTMWGHAYDAVTTKGGKYVQGGGCLTVGVPGLIQSGGFGSFSKHYGLAA
SSLLEAEVVTADGRIRIANARTEPDLFWALKGGGGGSYGVVSKVTLRLHDLPEFFGA
ATFTVKADSDEAYRRLIREFVGFYHEHLFDDHWGETVHVKPDNSLVVGMSSLGLDT
GQARRIWQPFLDWVANSGRACSVEGRAVIASLPARRFWDLQWWREHWPELGFPPN
GRRVIAWFDAVLAYVLRQPLFEIDPRPGAAPNNAWWTANAGEVAAFLWAYESLWL
PASLLEKDAQQPLADALFAASRYSAIELHFNKGLAGAPSAALSGAADTATNPAVLSA
FALAIAARGQGPAYPGIAGHEPSVAEARKAAERIGRCMSQLRALVPTPAAYVSESNY
FEQGWQRSYWGSNHPRLAAIKRRYDPDGLFFVHNGVGSEQWSADGFTKL (SEQ ID
NO: 77).
>BBE40.1
FGFELPAITRTGAETTITHAEVKELAKSLTLALLTPDSHRYHENRKIW
NGMWDDKHPALIVQCMDVSDVINAVNFARTHDLLLAVRGGGHSISGKSVCHGGMV
IDLSYMRSVQIDPYRKTARVGGGALLSDLDRAAQRFGLVTPSGVVSHTGVAGLTLGG
GIGRLMRKYGLTIDNLLSVEIVTPDGRLQRASDQENADLFWAVRGGGGNFGVVTAF
EFRLHSMGTEILSCGLAYPLDQAKDVFKFYFDFSREMPDELHFGLSAAIQENGDSVGL
FFGLGYSGSLKEAERIIEPIRKFGKPSFEAIGMRNFVELQSMGDAHNEHGRTYYIKGR
HMNDFDPDMVDPFIEQFQHAPSRFTVMRIVRFGGAIERVAKDATAWPHRDAKWDFE
VGGSWPNPDLSEKYVQWGRDYWESLDSYMADSFYVNELMDEGQSHVNANYQGNY
KRLSKLKQKYDPKNLFRLNANIEPSG (SEQ ID NO: 78).
>BBE41.1
LPLAADPTGPKDCAGILPPLSALAGVPAAVAEPNWAQRAGTINDASC
LNRTPVAGVVAVRNEADVARALAYARAHKLTVTAAGMKHSMGGQAFRAGGVILD
MRGLNAIRLDANTRTVSVGSGATWHAIQNAIHPQFAVKAMQSTDIFTVGGSISVNAH
GMDHQSGAVMGSIRSLRLMLADGRIVTASRTENADLFAHVVGGYGLFGVLLSAELE
VVPNDVYRSERSLISYRDFPETFAAIEADPAIGLTYTHLSTAPGSLLQEALVYAYRKVP
ADQSLPRAPLGEVSSTKLRRLTVNLSKRNDFFKAAKWWAEKNVEHRLESCTIPRAQA
MGDGEACLVSRNDPMHDSVAYLRDALPRETDILHEYFVPRDRLVPFIDGMRALFRQ
QDVNLVNASIRAVGAEVNALSYAPKPAFSVVLYLNQPADAGGTAAMARLTSDLIDL
CDREGGRFFLPYQLHYTTAQLVRSYPELPAFLAEKRRWDPDGLFGNTWYARYAPAV
TGLQTG (SEQ ID NO: 79).
>BBE42.1
DHRIAPWDELASKLQGALVRPGAASFSELSTPWALQYASTIPQAIAQC
RTEADVQTCIQWAKANSMPIVARSGGHSYGGYSLTTGLMINVSALNTFTYEPTTGLA
TVGSGARNRTIFTQCKPLDRAIPHGRCYEVGVAGLVLGGGIGFDMRLHGLTCDALRE
TRIVLADGRMLTCNEKENADLFWACRGAGGGNFGIHTSFTFDTFPVGTITTFNLVWT
DRLADTFAAIQTMTLSAPDTLGIKVSITAQKQAGASVLSLSILGHFVGSEADLRSLIAPI
LTVQKPAEQTIQIAPYWDGQQLITEEGRPEYSHERSRFVKGQLSDEAVRQIILNLKNW
PGTSKAATWKYFLLGGQIDARSPTDMAFVHRGYSMLSSIELEWTPADDAALLAKNE
QWLTGFHNQMEAYTSSYCYQNFIDPSQEGYLQAYYGTNLPRLQAIKRKYDPENLEN
YPQSIPV (SEQ ID NO: 80).
>BBE43.1
MKWKSKKRKDAELRVAGWDALPSVDLLFSDKDAHTLRALRAQKPIP
LDQRFSVDDVKDLQKAIHGTVVLPTDLTYHRDRQMTLLAVQEFPQLIVFCEIFEDAR
RCLEFAQKYDLRVTIRSGGHSTAGFSVNSGMLIDTSRIKHVVVDPKEKRAIVGAGATI
GDLNAALKMYNLHVPSGGCGDVCIAGYMQGGGYGFTARKFGMNCDNVHEIVVML
ADRTIVVANSEKNADLFWAVRGGTGGNFGILLQVTYNLHELGDVWGFGLRWSIDSR
DGIKNAAAALAEMQANYMQTGAPPELGFMTFLAWQDETPCVVMRGMYLGPQRDG
EFAIDSLRRLGPVYTDFGVGRYADLDLKLIESPPGLPQVPDLAREEKQCGYIDKKLDA
EDWAHIIGVFLTSPNRSSLVCIEPYGGRINEIPATSNAFIHRKVDMNLFLDVFWMSEDQ
QRKDAIAFLDRFMAMMAPYFNDHSYQNYPRRTQQDFRRRYWGSSFERLLAVKLKY
DKNSFFQYAQSVSRETPETHYDLPQLPEGIETEPY (SEQ ID NO: 81).
>BBE44.1
MEHPLRVDEFLPSDAPVPAADAEIAAPPAEGVAPLPPLPNLPVTDVEF
LRPQDAHYADYLPAASKRKQLAPALRAVCKTEHAVAVMVDWVRSNGLSFAVRCG
GHSYEGLSQSSGVAIDVRGLKQIVVDKASNLVTAGSGVSLYELYSALAAQGLALQA
GSCPTVGISGHLTGGGHGLLARSHGLTCDGLLQANVVDTQARVLQANATSEPDLWW
ACRGGGGGSFGIATEFRIEVFPLKTTRVFGVSWKLSQAHAAQLFAAWQDWAPNAPS
NITSIMKVGPAGHGLITMRCIGQSVGGESELRSELRRLTAVRPPSSALSVQSLAFLDAV
KHFAGPLAYESVLMKAKSDYVLTPLASDGIEAMMAAVAPIVSGGIVLLCDSYGGRIA
DVAADATAFPRRAGTQYCIQYFSSWSRSADTAAHLADVARVYAAMRPFMPGASYV
NYCDLDLDDYASAYWGDNLARLVAVKQQYDPDDLFHHAQSVPLSVPTA (SEQ ID
NO: 82).
>BBE45.1
MSGIRRRPETDESCRTAPAATEDLPMTATPASSPPENTERLTVRTRTS
VLAQLRDRIAGDVYGPGDAGYVDARSGFSRLDVHRPAVIAVPANHFDVVEAVRFAV
EEDLRVAVQATGHGTGRPADGGLLINTSRLASVSVDPVTRTARVGAGATWRTVVDA
ATPYGLAPLMGSASGVGAIGYTLGGGFGWLGRRFGLASDHVLSFDLVTPDGTPIRVT
DTSFPHIFWALKGAGAGGLGVVTSMEISLVEVSTVYAGNLFYPAADAEEIIRRFRDW
APAQSDALTSAVAILNLPPTDDVPEPLRGRSFAVVRGCWSGDLDAGRAVVDEWRDW
RTPVVDMWDVLPFAAIDAVSMDPTEPMPVMVTTEWFDDLPDVAVDIVAARVRPAP
ASAPLIVIGEIRHAGGAIARGAEHAPNDRARTGRFLLELVAAVPDPQVALAVESTLRV
TRTELAPYVTGAAYLNFTSGTERVARADDAYSSDHRRRLDEIKAALDPSNRFCHGDI
(SEQ ID NO: 83).
>BBE46.1
MLALQNGNQFKKHPMEKITANDPRYPDLAGKRFNKRFSGKPEYICLP
TTTVQVVEAVQEAVDNNKRLVVRSGGHCLEGFVTDPAIQVVLDLSLMTNIAYDTDK
SAFMVEAGATVGEMYRRLFLGWGVVVPAGEHPGIGVGGHVVGGAFGFICREYGLA
ADYLYGVEVVTVNTSGKAQRAIATRNTNDPNRELWWAHTGGGGGNFGIVTRYWFR
TPLNTGSDPFTALPPAPAAVTTFRVAWDWKNIDKASFTHLIRYFGEWCQQNSGPDSP
YRQLFSLLFLNHRNIGTLEMKGGATGPAAKQLIEEHLAAIADPLGLSYTPQIEELPWL
RFALNPFPELFQPGFDDAKAKIKDALLRRPLDDSQIATAYKYLTMEGNMGVLGMAT
YGGKVNAIAPAITASAQRDCILDIACNTGWGDPAEEASAVAWVRHFYRELFAHTGG
VPVPNEQAGGAMINHPDTDLADPEWNKSGVPWHALYYKENYPRLQKVKNQWDPL
NIFHHALSIKAVDQ (SEQ ID NO: 84).
>CsCHAP1
MGSLRRALVSADRLLQSSAAIHRRVSIAAANSLVRAPESLCRPRSSVA
YSGVYRFYSNNYTNLQSLDIDLSNEENKKRLYNRLVYRSKQRGFLELDLVLGKWVE
EHISSMDENGIIALVHVLDLENPDLWKWITGQEQPPEALQINPVFNALRDKVMNNLN
THSSPETRAKPGQPWVRGWDDIKRARDSPITGNQ (SEQ ID NO: 85).
>CsCHAP2
MEKMIPLRSLYRSLWTRSYHMAALNHNGLRHYYAARCLPNFSSPSTS
VTSRQALVSSDCRSPLSLSMGSTRSYSEDVTHMPVVTDPDLNRAFKDLLAASWDDLP
DSLVHEVKAALSKNTDDNAGKEALTNVFRAAVASEEFGEIIMNLKMEFDDTVGMSG
ENIKPLSKEHANALQTIFSRYNTYLEAFGPDETYLRKKVETELGSKMIYLKMRCGGLS
SDWGKVSVLGTSGLAGSYVEKRA (SEQ ID NO: 86).
>CsCHAP3
MGSLRRALVSIDRLLQSSAAIHRTISIAAANSLVRAPESFCRPRSSVAY
SGGYRFYSNNYTNLQSLDIDLSNEENKKRLYNRSKQRGFLELDLVLGKWVEEHISSM
DENGIKALVHVLDLENPDLWKWITGQEQPPEALQINPVFNALRDKVMNNLNTHSSPE
TRAKPGQPWVRGWDDIKRARDSPITGNQ (SEQ ID NO: 87).
>XP_030493922.1_FAD synthase [Cannabis sativa]
MEIDKAIRESDDRWLKTKYNKAIYVIQRALALYSIEEVAFSFNGGKDS
TVLLHLLRAGYFLHKGGQNCANGGPSDFPIRTIYFESSSAFPEINSFTYDAAANYGLQ
MDIIRTDFKSGLEALLKANPIRAIFLGVRIGDPTAVGQEQFSPSSVGWPPFMRVNPILD
WSYRDVWAFLLACKVQYCSLYDQGYTSIGSIHDTVPNALLSVSNSLSSEEKFRPAYL
LSDGRLERAGRVNKFSSGSGHSPSFSNGLISADLHKNSLLTASAIAVGDEILLGIVEDQ
LGPLLCKKLHSIGWSMSQTVVVRNDVDSVAEEVERKSSTNDLVFIYGGIGPLHSDLTL
AGVAKAFGVRLAPDEEFEEYLRSVIGDYCPSDRNEMALLPEGITELLHHEKLAVPLIK
CKNIIVLTATNFMELETQWDCLIELTRSSDILAVMEPYVSRLLKTDLSDVATAKPLSK
LCLEFPDLHISCYRQTRYGPLIVNIRGRDEARIEAAVEALSKKLHPGAFSEINSS (SEQ
ID NO: 88).
>XP_030510352.1_dnaJ 49 [Cannabis sativa]
MDGNKEDALKCLRIGKEAMEAGDRNRARKFFTKAQRLDPTLSVDDL
LSAVEKESGGERSPSDADASSNGPSDSTSSKPSDQPSLRQRVPSTGSSSSPSSATGAHT
LEQIAIVREIKKKKDYYEILGVEKSCSVEDVRKAYRKLSLKVHPDKNKAPGAEEAFKS
VSKAFQCLSSEESRRKYDLVGSDEPVYERRTTRHAAHGFNGFYDADVDAEEIFRNFF
FGGMAPATQFRGFSFGTGMGHRQQQPDHNGSGGFNVRALIQILPVLLILVLNFLPSSE
PIYSLSRSYPYEYKFTTQKGVNYYVKSTKFEQDYPLDSRERSSFEDQVEREYFGILNQ
NCRLEIQRRQWGFIRETPHCDMLQRFQTQAN (SEQ ID NO: 89).
>XP_030489210.1_ClpB1 [Cannabis sativa]
MNPDKFTHKTNEAIAAAHELAMSGSHAQFTPLHLAVSLINDPSGIFSQ
AVANASGSEDAPKSVERVFSQALKKLPSQSPAPDDIPASTSLIKVIRRAQAAQKSRGD
THLAVDQLILGLLEDSQIGDLLKEAGVATARVKSEVEKLRGKDGKKVESASGDTNFQ
ALKTYGRDLVEQAGKLDPVIGRDEEIRRVVRILSRRTKNNPVLIGEPGVGKTAVVEGL
AQRIVRGDVPSNLADVRLIALDMGALVAGAKYRGEFEERLKSVLKEVEEAEGKVILF
IDEIHLVLGAGRTEGSMDAANLFKPMLARGQLRCIGATTLEEYRKYVEKDAAFERRF
QQVYVAEPSVPDTISILRGLKERYEGHHGVRIQDRALVIAAQLSSRYITGRHLPDKAID
LVDEACANVRVQLDSQPEEIDNLERKRMQLEVELHALEKEKDKASKARLVEVRKEL
DDLRDKLQPLMMKYRKEKERIDEIRRLKQKREELLIALQEAERRYDLARAADLRYG
AIQEVESAIAQLEGTTDENLMLTETVGPDQIAEVVSRWTGIPVTRLGQDDKDRLIGLA
DRLHKRVVGQDQAVAAVAEAVLRSRAGLGRPQQPTGSFLFLGPTGVGKTELAKALA
EQLFDDENLLVRIDMSEYMEQHSVSRLIGAPPGYIGHEEGGQLTEAVRRRPYSVVLFD
EVEKAHVSVENTLLQVLDDGRL (SEQ ID NO: 90).
>SRP155904_DN9237
MMNVQMGDVETFAFQAEINQLLSLIINAFYSNKEIILRELISNSSDALD
KIRFEGLTDKSKLDAQPELFIRLVPDKANKTLSIIDSGIGMTKADLVNNLGTIARSGTIE
FMEALQAGADVSMIGQFGVGFYSAYLVAEKVIVTTKHNDDEQYIWESQAGGSFTVT
RDVDGEQLGRGTKITLFLKEDQLEYLEERRIKDLVKKHSEFISYPIYLWTEKTTEKEIS
DDEDEGTKKEEEGDIEEVDEEKEKDSKKKKKVKEVSHEWQLINKQKPIWLRKPEEIT
KDEYASFYKSITNDWEDHLAVKHFSVEGQLEFKAILFVPKRAPFDLFDTRKKMNNIK
LYVRRVFIMDNCEELIPEYLGFIKGVVDSDDLPLNISREMLQQNKILKVIRKNLVKKCI
EMFNEIAENKEDYAKFYDAFSKNLKLGIHEDSQNRAKLADLLRYHSTKSGDEMTSLK
DYVTRMKEGQKDIFYITGESKKAVENSPFLERLKKRGYEVLYMVDAIDEYAVGQLK
EYDGKKLVSATKEGLKLDDETEEEKKKREEKKKSFENLCKTIKDILGDKVEKVVVSD
RIVDSPCCLVTGEYGWTANMERIMKAQALRDSSMSSYMSSKKTMEINPDNGIMEEL
RKRAEADKNDKSVKDLVLLLFETALLTSGFSLDDPNTFAARIHRMLKLGLSIDEEEVE
GDEVEMPPLEEDGNEESKMEEVD (SEQ ID NO: 91).
>SP3
MKSLLLSLLAVPATA (SEQ ID NO: 92).
>SP4
MKFSAVSIAAALASLVAA (SEQ ID NO: 93).
>SP7
MKLSTILFTACATLALALA (SEQ ID NO: 94).
>SP8
MKVLALLVTVCFSVASA (SEQ ID NO: 95).
>SP11
MIFDGTTMSIAIGLLSTLGIGAEA (SEQ ID NO: 96).
>SP12, SP-KAR2
MKFSMPSWGVVFYALLVCLLPFLSKAGVQA (SEQ ID NO: 97).
Lip2-THCAS
MKLSTILFTACATLAAALPSPITPSEAAVLQKRVYTSTETSHIDQESYN
FFEKYARLANIGYCVGPGTKIFKPFNCGLQCAHFPNVELIEEFHDPRLIFDVSGYLAVD
HASKQIYLVIRGTHSLEDVITDIRIMQAPLTNFDLAANISSTATCDDCLVHNGFIQSYN
NTYNQIGPKLDSVIEQYPDYQIAVTGHSLGGAAALLFGINLKVNGHDPLVVTLGQPIV
GNAGFANWVDKLFFGQENPDVSKVSKDRKLYRITHRGDIVPQVPFWDGYQHCSGEV
FIDWPLIHPPLSNVVMCQGQSNKQCSAGNTLLQQVNVIGNHLQYFVTEGVCGIGHHH
HHHMDSPDLENLYFQSNPRENFLKCFSKHIPNNVANPKLVYTQHDQLYMSILNSTIQ
NLRFISDTTPKPLVIVTPSNNSHIQATILCSKKVGLQIRTRSGGHDAEGMSYISQVPFVV
VDLRNMHSIKIDVHSQTAWVEAGATLGEVYYWINEKNENLSFPGGYCPTVGVGGHF
SGGGYGALMRNYGLAADNIIDAHLVNVDGKVLDRKSMGEDLFWAIRGGGGENFGII
AAWKIKLVAVPSKSTIFSVKKNMEIHGLVKLFNKWQNIAYKYDKDLVLMTHFITKNI
TDNHGKNKTTVHGYFSSIFHGGVDSLVDLMNKSFPELGIKKTDCKEFSWIDTTIFYSG
VVNFNTANFKKEILLDRSAGKKTAFSIKLDYVKKPIPETAMVKILEKLYEEDVGAGM
YVLYPYGGIMEEISESAIPFPHRAGIMYELWYTASWEKQEDNEKHINWVRSVYNFTT
PYVSQNPRLAYLNYRDLDLGKTNHASPNNYTQARIWGEKYFGKNFNRLVKVKTKV
DPNNFFRNEQSIPPLPPHHHHHH (SEQ ID NO: 98).
THCAS-CWP1
MNPRENFLKCFSKHIPNNVANPKLVYTQHDQLYMSILNSTIQNLRFIS
DTTPKPLVIVTPSNNSHIQATILCSKKVGLQIRTRSGGHDAEGMSYISQVPFVVVDLRN
MHSIKIDVHSQTAWVEAGATLGEVYYWINEKNENLSFPGGYCPTVGVGGHFSGGGY
GALMRNYGLAADNIIDAHLVNVDGKVLDRKSMGEDLFWAIRGGGGENFGIIAAWKI
KLVAVPSKSTIFSVKKNMEIHGLVKLFNKWQNIAYKYDKDLVLMTHFITKNITDNHG
KNKTTVHGYFSSIFHGGVDSLVDLMNKSFPELGIKKTDCKEFSWIDTTIFYSGVVNEN
TANFKKEILLDRSAGKKTAFSIKLDYVKKPIPETAMVKILEKLYEEDVGAGMYVLYP
YGGIMEEISESAIPFPHRAGIMYELWYTASWEKQEDNEKHINWVRSVYNFTTPYVSQ
NPRLAYLNYRDLDLGKTNHASPNNYTQARIWGEKYFGKNFNRLVKVKTKVDPNNFF
RNEQSIPPLPPHHHDDDDKMKFTAATLLLAAAASALDVVTDGSKPFGIMSLRSASAIH
LSSVGVSGDALTIGSSGAKFTIKDGVLYADDKAIDFSSGEAKVASDGKGTSGVTLEK
GYVTVPGFNWAGCPEGNGYAVDDNSKCEDDGIPFGAYAVADTSAESSAAPASSAAA
AESSAAPSSAAEAKPTAGGNTGAVVTQIGDGQIQAPPSAPPAAPEQANGAVSVGVSA
AALGVAAAALLI (SEQ ID NO: 99).
>LIP2
MKLSTILFTACATLAAALPSPITPSEAAVLQKRVYTSTETSHIDQESYN
FFEKYARLANIGYCVGPGTKIFKPFNCGLQCAHFPNVELIEEFHDPRLIFDVSGYLAVD
HASKQIYLVIRGTHSLEDVITDIRIMQAPLTNFDLAANISSTATCDDCLVHNGFIQSYN
NTYNQIGPKLDSVIEQYPDYQIAVTGHSLGGAAALLFGINLKVNGHDPLVVTLGQPIV
GNAGFANWVDKLFFGQENPDVSKVSKDRKLYRITHRGDIVPQVPFWDGYQHCSGEV
FIDWPLIHPPLSNVVMCQGQSNKQCSAGNTLLQQVNVIGNHLQYFVTEGVCGI (SEQ
ID NO: 100).
>LIP2_linker
GHHHHHHMDSPDLENLYFQS (SEQ ID NO: 101).
>EK_cleavage
DDDDK (SEQ ID NO: 102).
>CWP1
MKFTAATLLLAAAASALDVVTDGSKPFGIMSLRSASAIHLSSVGVSG
DALTIGSSGAKFTIKDGVLYADDKAIDESSGEAKVASDGKGTSGVTLEKGYVTVPGF
NWAGCPEGNGYAVDDNSKCEDDGIPFGAYAVADTSAESSAAPASSAAAAESSAAPS
SAAEAKPTAGGNTGAVVTQIGDGQIQAPPSAPPAAPEQANGAVSVGVSAAALGVAA
AALLI (SEQ ID NO: 103).
>FADS1a
MIVGPKTKFHSLYNHMFSQTCHKFSGVVDSFLASNGGALRKATQDH
VRESLRVLHEALERYDLNQISLSYNGGKDCQVMVILLLAALWRRFGEDPAVLNNLG
AFKSVYVASEKAFEEVDTFVDNSCQEYGLQQIRLSEPMKAAFEHFLSENPTVKAIIVG
IRRSDPYGQQLKPFDPTDSGWPDFMRVHPVLEWKYVNIWDFLRGTDSAYCCLYDKG
YTSLGGTDSTVPNPKLLKKGTCAEFLPAYALVKDEEERLGRFRK (SEQ ID NO: 104).
>HAC1s
MSIKREESFTPTPEDLGSPLTADSPGSPESGDKRKKDLTLPLPAGALPP
RKRAKTENEKEQRRIERIMRNRQAAHASREKKRRHLEDLEKKCSELSSENNDLHHQV
TESKKTNMHLMEQHYSLVAKLQQLSSLVNMAKSSGALAGVDVPDMSDVSMAPKLE
MPTAAPSQPMGLASAPTLFNHDNETVVPDSPIVKTEEVDSTNFLLHTESSSPPELAEST
GSGSPSSTLSCDETDYLVDRARHPAASAQDFIFIKDEPVDDELGLHGLSDDFTLFEDN
KQPAQHDFIADLAHYESSVSNLFGGLE (SEQ ID NO: 105).
BBE47
MNEADEVNEVDELSETGQTSGTKGKRPFTGRVIGPADGEFDEARRV
WNECFAARPKEIVYCADTRDVVRALREVRQRGGPFRVRSGGHSMSGLSVLDDGTVL
DVSGLDDIQVSEDASTVTVGSGAHLGDIFRALWARGVTVPAGTCPEIGIAGHVLGGG
AGILVRSRGFLSDHLVALEMVDSEGRIVVADHDSHHELLWASRGGGGGNFGIATSFT
LRTQPIGDVTLFTIAWDWDRGAEAIKAWQEWLATADGRINTLFIAYPQDQDMFAAL
GCFEGDAAELEPLIAPLVHAVEPTEKVAETMPWIEALSFVETMQGEAMKATSVRAK
GNLSFVTEPLGDRAVEEIKKALAQAPSHRAEVVLYGLGGAVAAKGRRETAFVHRDA
PVALNYHTDWDDEAEDDLNFAWIQNLRASVAAHTEGRGSYVNTIDLTVEHWLWDY
YEENLPRLMAVKKRYDPEDVFRHPQSIPVSLTEAEAAELGIPPHIAEELRAARQLR
(SEQ ID NO: 106).
BBE48
MATPSAFSGSVLTPGDDGFEAAQVTWNACYSSRPREVMVCHDAASV
AEAVRSVRERGLPFRVRSGGHSMCGLSNLDDGVIIDLGGLGGVELTPDRQTVRIGGG
ARLADVYNTLWDHRLTVPAGTCPRIGVGGHVLGGGMGVLSRSRGALVDHLTALEM
VDAEGRLLRVSEDENPDLFWACRGGGGGNFGIVTAYELRPTPIDDVTIFTVSWTWSQ
LPDAVRAWQRWLGSAESRINSFLSLFPQQQDMVVAFGVFDGPAADFRPLLAPLTAEV
APEAEVVEEVPFIQAVDTVEALQGEAAAAEQVRAQGSSAIIANPLNDEALATLQEFLT
DPPSHRAEVAVYGMGGVIGERERGDTAFVHRTGLMAFNYRTDWDSPEDDRLNLDW
VTRLRHAMAEHTTGAAYVNTIDLALENWLWAYYEENLPRLMAVKRRYDPENVFHH
PHSIPGSLTAEAARAHGVPEATLKRLHDDGLLDGPLD (SEQ ID NO: 107).
BBE49
ATSARFAGTVLTPGDPGFEDASRTWNACFASRPREVMVCHSTAAVA
DALREVRERGVPFRVRSGGHSMGGLSSVEDGVVIDLGGLDSISLSEDRKLVTVGGGT
RLGDIYRELWRFGVTVPAGTCPRIGIGGHVLGGGMGILSRSRGALVDHLAALEFVDA
EGRTYTVDENEHPDLFWACRGGGGGSYGIATSYTLRTSPVDEVTLFTFSWSWDQFAP
AVRAWQSWLASTDDRVNTFLSLFPREQDLVAAFGVYDGPAAEFEELMRPLVSAVPP
QDQVVEPMPFIQAVDTVEALQGEAVAADQVRAQGSSAIVAEPADDAAIATLQTFLTD
PPSHRAEVAVYGMGGAIGRRARTDMAFVHRTGLMAFEYRTDWDRPEDDGKNLAW
VEALRHAMAPHTTGAAYVNTIDMALENWLWAYYEENLPRLMEVKRRYDPGDVFH
HAQSIPLSLTEADIARYGIPAPVAARLRAAGLTSDQEH (SEQ ID NO: 108).
BBE50
MVTRSAHVAATVTAADPGYDEARRVWNERFDCRPRAVLYPADVAE
VTVAIRAARDEGVPFRIRSGGHNVEGFSVLDDGYVIDLSRLTGVRVSDDRARGTVVA
GTLLGDVYSGLWAAGVTIPAGVCPDIRIGGHVLGGGVGMLVRSRGMLIDSLVGLTM
VDADGEVLEADDDSHPDLMWASRGGGGGNFGVATSYTFHTRPIGDVTLVTATWDS
MNAITALDRWQHWMVDADKRVNARFSLFSPATGGGLMAALFEGTRAEWESLARPL
LAQCPPTDYGIRETAYINSVGTFSQLVPSIRAKFVPALAAGPLDAEALKILERRHRDAP
DGVKTGFYGLGGAVLGAGLAEKSAFAHRSSLFCVEYLGHWKGAENDAEHLGWLAG
IRDELRPFMTGGAYVNSPDRDLSDWLHAYYGASLPRLMDVKRRYDPEDVFRFEQSIP
VSLSPSAARAGGLTAPVIADLSARGLLT (SEQ ID NO: 109).
BBE51
MATGAAVVAGDAVADARPARWARLRAHLAGELLLPGDDGYDTAR
LPFNSLFDHHRPAGIARCVRPEDVQRCLEEARRSGIRVAARSGGHGYAAYSTPHDGL
VVDLAPMAGVEVRPDGTAVVGAGARLGDVYAALAAAGRCLPLGNCPSVGIAGFTL
GGGLGWLQRTYGLACDWLRAARVITVDSRVVTASEDREPDLFWALRGGGGGNFGV
VTSFGFATVPVPEVLTVFRVRFPAGSAADVLGGWQPWSVALPEEVSSSCVAMPDGA
VDVFGCAVGREGMVLGELAHLLDAVGVDAPIEHAEVDYPTATAIMTGGQPTRNAFR
ASSRIAERWDDPAAVADTVRGMDSALLIVGALGGAIDRRTPTDTAYPHRGAPTSVEV
FGTVGDLPPEQVTREVDQAQSALAGLIGTGTYINYLDPEQQDWARTAYRHNLRRLR
AVARRYDPDRVLRFPQAVR (SEQ ID NO: 110).
BBE52
MEKTKLTGRIVTRDDPGYNDARTNINLSLQRYPRLIVFCQNKHDAVN
ALKWARENNVPFRIRGGRHSYENFSLLNNGLVIDLSEMNRIKVDHNKQLATIEAGAE
LGEVYRTLWRYGLTLPAGNIANVGLTGLTLGGGIGYLTRSAGLTCDHLVKLEMITAE
GKEEAELITVSRSEHPDLFWASQGGGGGNFGIVTSMTFKAVPISRVSIFSITWGWKDF
EEVENTWQRWAPFTDERLTSSIEFWPEEVNRIEALGQFVGTKTELKELLKPLLKAGKP
TSGIVKTVPFIEAAAFFNSPGGNRPQNLKRSGSFIEKPLSNHAISTIKHFLQQAPNQNAS
VWQQSLGGAAGHIAPDETAFFYRDAIIAQEYLTNWTSPEEKSQNVRWIEGLRTALSK
ETMGDYVNWPDLAIRNWQKTYYGENFERLREVKTKYDPDNVFRFEQSIPPLRRSIFF
(SEQ ID NO: 111).
BBE53
MPAARILRRITRSDREFEALCRGWNRRFHADPLEIVFPASTDEVAAAL
LETQERGIPFRIRSGGHSVDGFSGIADGIVIDLRDLNTVDISADRTHVRVGAGAHLHD
VYEVLGQIDRVIPGGVGRSVGMGGLITGGGLGGLTRWLGALAHNVVSFDLVDAQGE
IHRVTPDSHPDLYWACLGAGGGNFGIITAFTLRMTPLSAAVWYRLSWPWSQFEDVY
GAWQQWAPTADSRLTPLLVMWPTTSANRVDVAGIFPGTPEQLEDLLEPLLESVPPPS
ERQITPTTFVAAMAALLEKIGGESFGVGTRAVTASPFFDRPLDRNLMRILRDWHTGAP
GNSFVWCLPGGAELATAAARGMQSFAHGQMQQLMLIRSDWTDVREDDACIAWVS
GLYELMQPYACGAYLNWTNTDIESRPHRLYRDSYARLVQIKHRYDPNSIFSFTHAIPA
SISRVQARQLQLPDSTLAELSHRGVLVD (SEQ ID NO: 112).
BBE54
MRTRTLARLESLRDAVRGSVLLPGDAGYDQERTGFQSGDRHGPDVL
VAARTPEDVRAAVDHARRRGMPLAVQATGHGLRAAVREGVLVGTRRMTGIRIDAA
ARTAWVEPGVRAGDLVAAAARHGLAPVNGSAPGVGVVGYTLGGGVGLLGREFGY
AADLVRRLDVVTADARLRRVTAERCPDLFWGLRGGGGGLGAVTGLELGLVPVERFF
GGRLVFDGGQVAEEVLRTWQEWTRSVPDTLTSAVTLLPMPDLPAVPAALRGRYLAQ
VHLAFTGPREEGERLVRPLREIGALLVDEVRERPYTECATVFSEPETPHAYRSSNVLL
GAAGLDDGARRAVPAEAGPSAAAMCVLGVRHLGGAFSRPPEVPNAVDHRDARYLV
YVLSPVPEPDRDGAVRSLHARVLAPVTGPAPARSGNFLHGPQEPATLRSVHGDETAA
RLERLRSAYDPQGLFRGIGRFAGPG (SEQ ID NO: 113).
BBE55
MNDMSLTNLQAGKTTISAAAIEALAGRLRGRVLDARDPAYDEARTI
WNAMVDRRPGLIVQAAGASDVINAVRFAAENQLLVAVRGGGHNIAGNAVCDGGL
MIDLSPMKSVRVDQTTKRAWVEPGATLADVDKETQAFGLALPTGINSTTGIAGLTLG
GGFGWTTRKLGLTIDNLLSADVVTANGELVRASQTEHRDLFWGLRGGGGNFGVVT
AFEFQLHELGPEVLAGLVVHPFADAENVLRQYRQALENAPDELTCWAVMRQAPPLP
FLPEEWHGREILALAMCYCGDLEAGQKATAGLRGIGKPIADVVGPAPFAAWQQAFD
PLLAPGARNYWKSHDFMELSDQTIGILTDAVRQLPGPECEIFVGHVGGAAGRVAAEE
TAFPQRSSHFVMNVHARWREPQMDQACIEWARRLFEAAKPHAVGTAYINFMPEDEG
DRVEAAYAGNYRRLLEVKGRYDPQNLFRMNQNVRPAGLRGAA (SEQ ID NO: 114).
BBE56
MATSARFAGTVLAPGDPGFEDASRTWNACFASRPREVMVCHSTAEV
ADAVREVRERGVSFRVRSGGHSMAGLSSVEDGVVIDLGGLDAISLSADRSLVTVGGG
TRLGDIYRELWRSGVTVPAGTCPRIGIGGHVLGGGMGVLSRSRGALVDHLAALEFVD
AEGRAYRVDENEHPDLFWACRGGGGGSYGIATSYTLRTSPVDEVTLFTLSWNWERF
APAVRAWQSWLASTDDRVNTFLSLFPRQQDLVAAFGVYDGPAAEFEELLRPLVAAV
RPQDQVVEPMPFIQAVDTVELLQGEAAAADQVRAQGSSAIIGGPLDGAAIATLQQFL
TDPPSHRSEVAVYGMGGAIARPARTDTAFVHRTGLMAFEYRTDWDRSEDDAKNLA
WIKALRHAMAPHTTGAAYVNTIDLALENWLWAYYEENLPRLIEVKRRYDPGDVFH
HPHSIPLTVTEADIARYGIPEAVAARLRAAGLTDRQ (SEQ ID NO: 115).
BBE57
MATSARFAGTVLAPGDPGFEDASRTWNACFASRPREVMVCHSTAEV
ADAVREVRERGVSFRVRSGGHSMAGLSSVEDGVVIDLGGLDAISLSADRSLVTVGGG
TRLGDIYRELWRSGVTVPAGTCPRIGIGGHVLGGGMGVLSRSRGALVDHLAALEFVD
AEGRAYRVDENEHPDLFWACRGGGGGSYGIATSYTLRTSPVDEVTLFTLSWNWERF
APAVRAWQSWLASTDDRVNTFLSLFPRQQDLVAAFGVYDGPAAEFEELLRPLVAAV
RPQDQVVEPMPFIQAVDTVELLQGEAAAADQVRAQGSSAIIGGPLDGAAIATLQQFL
TDPPSHRSEVAVYGMGGAIARPARTDTAFVHRTGLMAFNYRTDWDRSEDDAKNLA
WIKALRHAMAPHTTGAAYVNTIDLALENWLWAYYEENLPRLIEVKRRYDPGDVFH
HPHSIPLTVTEADIARYGIPEAVAARLRAAGLTDRQ (SEQ ID NO: 116).
BBE58
MSTAPVFGGRIVTRLDPSYEEARQVWNAVPDHRPLEIRYCRDAQDV
AAALRDALARGLPFAARSGGHSAAALSVVPDGVVIDVSPLNSISLDQDASQVTFGSG
ATLGAIYRTLWESGVTVPAGTCPNIGVAGHVLGGGLGILSRSRGPLVDHLIGVTMVD
ARGELVTADEENHSDLLWACRGGGGGNYGIATSFTLRTQPIADVTLFNITWRFDDLG
PAVKAWQSWLADADHRINPFMMLYPREQDMVATVGVMEGTPDAFEPLVKPLLDAV
EPSEVVIEPMSFIQAVDTVEALQGEAAVAKSVRALGSSAIIDRPLDDRALETLRRHLID
PPSHRSEVAIYGMGGKIAAKGREETAFVHRTELMAFEYRTDWDEPADDEKNVAWV
RSVRQEMAEHTTGGTYTNTVDLGVENWLWGYYEENLPRLMGVKHRYDPDGVFQH
AHSVPLSLTEAEASRFGIPEQIRRTLRAAGRLT (SEQ ID NO: 117).
BBE1.6
SEQ ID NO: 118
NPRENFLKCFSQYIPNNATNLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVCAGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHRH
BBE1.21
SEQ ID NO: 119
NPRENFLKCFSQYIPNNATNLKLVYTQNNPLYMSVLNSTIHNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVGVGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHRH
BBE1.22
SEQ ID NO: 120
NPRENFLKCFSQYIPNNATNLKLVYTQNNPLYMSVLNSTIQNLRFTSD
TTPKPLVIVTPSHVSHIQGTILCSKKVGLQIRTRSGGHDSEGMSYISQVPFVIVDLRNM
RSIKIDVHSQTAWVEAGATLGEVYYWVNEKNENLSLAAGYCPTVGVGGHFGGGGY
GPLMRNYGLAADNIIDAHLVNVHGKVLDRKSMGEDLFWALRGGGAESFGIIVAWKI
RLVAVPKSTMFSVKKIMEIHELVKLVNKWQNIAYKYDKDLLLMTHFITRNITDNQGK
NKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYG
GIMDEISESAIPFPHRAGILYELWYICSWEKQEDNEKHLNWIRNIYNFMTPYVSKNPR
LAYLNYRDLDIGINDPKNPNNYTQARIWGEKYFGKNFDRLVKVKTLVDPNNFFRNE
QSIPPLPRHRH
BBE2.16
SEQ ID NO: 121
NPRENFLKCFSKHIPNNATNPKLVYTQHDQLYMSILNSTIQNLRFISDT
TPKPLVIVTPSQNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFMTPYVSQN
PRLAYLNYRDLDLGKTNHASPNNYTQARIWGEKYFGKNFNRLVKVKTKVDPNNFFR
NEQSIPPLPPHHH
BBE2.18
SEQ ID NO: 122
NPRENFLKCFSKHIPNNATNPKLVYTQHDQLYMSILNSTIQNLRFISDT
TPKPLVIVTPSQNSHIQATILCSKKVGLQIRTRSGGHDAEGMSYISQVPFVVVDLRNM
HSIKIDVHSQTAWVEAGATLGEVYYWINEKNEQLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFMTPYVSQN
PRLAYLNYRDLDLGKTNHASPNNYTQARIWGEKYFGKNFNRLVKVKTKVDPNNFFR
NEQSIPPLPPHHH
BBE2.19
SEQ ID NO: 123
NPRENFLKCFSKHIPNNATNPKLVYTQHDQLYMSILNSTIQNLRFISDT
TPKPLVIVTPSQNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKQITDNHGK
NKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFMTPYVSQN
PRLAYLNYRDLDLGKTNHASPNNYTQARIWGEKYFGKNFNRLVKVKTKVDPNNFFR
NEQSIPPLPPHHHHHH
BBE2.20
SEQ ID NO: 124
NPRENFLKCFSKHIPNNATNPKLVYTQHDQLYMSILNSTIQNLRFISDT
TPKPLVIVTPSQNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
QKTTVHGYFSSIFHGGVDSLVDLMNKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFMTPYVSQN
PRLAYLNYRDLDLGKTNHASPNNYTQARIWGEKYFGKNFNRLVKVKTKVDPNNFFR
NEQSIPPLPPHHH
BBE2.21
SEQ ID NO: 125
NPRENFLKCFSKHIPNNATNPKLVYTQHDQLYMSILNSTIQNLRFISDT
TPKPLVIVTPSQNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMQKSFPELGIKKTDCKEFSWIDTTIFYSGVVNFNT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFMTPYVSQN
PRLAYLNYRDLDLGKTNHASPNNYTQARIWGEKYFGKNFNRLVKVKTKVDPNNFFR
NEQSIPPLPPHHH
BBE2.22
SEQ ID NO: 126
NPRENFLKCFSKHIPNNATNPKLVYTQHDQLYMSILNSTIQNLRFISDT
TPKPLVIVTPSQNSHIQATILCSKKVGLQIRTRSGGHDAEGMSYISQVPFVVVDLRNM
HSIKIDVHSQTAWVEAGATLGEVYYWINEKNENLSFPGGYCPTVGVGGHFSGGGYG
ALMRNYGLAADNIIDAHLVNVDGKVLDRKSMGEDLFWAIRGGGGENFGIIAAWKIK
LVAVPSKSTIFSVKKNMEIHGLVKLFNKWQNIAYKYDKDLVLMTHFITKNITDNHGK
NKTTVHGYFSSIFHGGVDSLVDLMNKSFPELGIKKTDCKEFSWIDTTIFYSGVVNENT
ANFKKEILLDRSAGKKTAFSIKLDYVKKPIPETAMVKILEKLYEEDVGAGMYVLYPY
GGIMEEISESAIPFPHRAGIMYELWYTASWEKQEDNEKHINWVRSVYNFMTPYVSQN
PRLAYLNYRDLDLGKTNHASPNQYTQARIWGEKYFGKNFNRLVKVKTKVDPNNFFR
NEQSIPPLPPHHH

1. A cell expressing an exogenous terminal cannabinoid synthase and overexpressing one or more chaperones, wherein the exogenous terminal cannabinoid synthase has at least one amino acid modification as compared to a wild type exogenous terminal cannabinoid synthase.

2. The cell of claim 1, wherein the exogenous terminal cannabinoid synthase is selected from i) a berberine bridge enzyme (BBE)-like family enzyme selected from the group consisting of BBE1.20, BBE1.21, BBE1.22, BBE2.6, BBE2.7, BBE2.8, BBE2.16, BBE2.18, BBE2.19, BBE2.20, BBE2.21, BBE2.22, and BBE2.14, having an amino acid sequence corresponding to SEQ ID NO: 19, 119, 120, 25-27, 121-126, and 33, respectively, ii) a berberine bridge enzyme-like enzyme comprising at least one amino acid modification compared to one of the amino acid sequences of BBE1.6 (SEQ ID NO: 118), BBE2.1 (SEQ ID NO: 20), BBE3.1 (SEQ ID NO: 34), BBE25.1 (SEQ ID NO: 59), BBE25.4 (SEQ ID NO: 62), and BBE25.5 (SEQ ID NO: 63), and iii) a functional fragment or derivative of any of the above having at least 70% sequence identity thereto.

3. (canceled)

4. The cell of claim 1, wherein the exogenous terminal cannabinoid synthase has improved solubility, stability, turnover, selectivity, Kₘ, or K_{cat} as compared to a wild type terminal cannabinoid synthase

5. The cell of claim 1, wherein the exogenous terminal cannabinoid synthase is preferentially expressed in a location selected from the cytoplasm, ER, golgi, liposome, vacuole, plasma or outer cell membrane, peroxisome, oleosome, and the extracellular environment.

6. The cell of claim 5, wherein preferential expression involves a synthetic, heterologous or native signal peptide, retention sequence, leader peptide, or sorting sequence.

7. The cell of claim 6, wherein the exogenous terminal cannabinoid synthase is expressed with a signal peptide selected from the group consisting of SP3, SP4, SP7, SP8, and SP11.

8. The cell of claim 1, wherein the exogenous terminal cannabinoid synthase is fused to a secreted protein or a membrane localization sequence.

9. The cell of claim 8 where the cannabinoid synthase is fused to lipase 2 (Lip2) having the amino acid sequence of SEQ ID NO: 100, cell wall protein 1 (CWP1) having the amino acid sequence of SEQ ID NO: 103, a 1,3-beta glucanosyltransferase selected from the group consisting of Uniprot Q6C8C9 and Q6CFU7), or a functional fragment of any of the foregoing having membrane localization or secretion activity.

10. The cell of claim 1, wherein the cell also over-expresses one or more chaperones selected from HAC1, HAC1s FADS1, FADS1a, KAR2, FMN1, CNE1, ERO1, PDI1, IRE, YAP1, HYR1, CsCHAP1, CsCHAP2, CsCHAP3, CsDNAJ, ClpB1, HSP90, or a functional fragment or derivative thereof.

11. The cell of claim 10, wherein the over-expressed one or more chaperones is HAC1, CNE1 or a functional fragment or derivative thereof.

12. (canceled)

13. The cell of claim 10, wherein the chaperones are overexpressed with a signal protein selected from SP3, SP7, SP8, and SP12, optionally with an HDEL motif.

14. The cell of claim 1, wherein the cell overexpresses one or more chaperones or homologs thereof involved in covalent attachment of FAD to terminal cannabinoid synthases and/or enzymes involved in FAD biosynthesis.

15. The cell of claim 14, wherein the cell expresses an exogenous FAD synthetase or a FMN synthetase, or over-expresses a native FAD synthetase or FMN synthetase.

16. The cell of claim 15 wherein the exogenous FAD synthetase is Uniprot ID Q6C7T3, FADS1, or FADS1a, and/or the FMN synthetase is Uniprot ID Q6CG11.

17. (canceled)

18. The cell of claim 1, wherein the expression of one or more proteases is inhibited or inactivated, wherein the one or more proteases inhibited or inactivated is selected from the group consisting of AXP1, XPR2, YALI0E33363p, YALI0E28875p, PEP4, PRB1A, PRB1B, YALI0E34331p, YALI0E29403p, YALI0E28875p, YALI0E26851p, YALI0E21868p, YALI0E13552p, YALI0E13233p, YALI0E05423p, YALI0E04829p, YALI0E02024p, YALI0F26411p, YALI0F21615p, YALI0F20592p, YALI0F19734p, YALI0F17974p, YALI0F16005p, YALI0F13585p, YALI0F11033p, YALI0F10769p, YALI0F07359p, YALI0F05940p, YALI0F01859p, YALI0F01540p, YALI0F00396p, YALI0F00176p, YALI0B20834p, YALI0B19228p, YALI0B17072p, YALI0B14641p, YALI0B13310p, YALI0B¹¹⁵⁹⁴p, YALI0B10934p, YALI0B05522p, YALI0C10648p, YALI0C10494p, YALI0C09438p, YALI0C08283p, YALI0C05280p, YALI0C02519p, YALI0C00165p, YALI0D04807p, YALI0D07920p, YALI0D10967p, YALI0D13046p, YALI0D15642p, YALI0D16335p, YALI0D18832p, YALI0D19910p, YALI0D22957p, YALI0D23309p, YALI0C21604p, YALI0B04158p, YALI0B02574p, YALI0B01386p, YALI0A13277p, YALI0A10615p, YALI0E14388p2, YALI0B03718p, YALI0B16500p, YALI0D10835p, YALI0F09163p, YALI0E22374p, YALI0C00803g, YALI0D02024p, YALI0F11803g, YALI0C20273p, YALI0B14641g, YALI0F11803g, YALI0C20273g and YALI0C10923p, and homologs and orthologs thereof.

19. The cell of claim 18, wherein the expression of YALI0F09163p, and/or homologs and/or orthologs thereof are inhibited or inactivated in the cell.

20. The cell of claim 1, wherein the cell has been modified to inactivate ROT2 glucosidase.

21. (canceled)

22. (canceled)

23. (canceled)

24. The cell of claim 1, wherein the cell has been engineered to enhance expression of the exogenous terminal cannabinoid synthase, wherein the engineering comprises one or more of:
a. improved import of the exogenous terminal cannabinoid synthase into a secretory pathway,
b. a modulated unfolded protein response,
c. a modulated disulfide bond formation activity,
d. a modulated FAD biosynthesis activity,
e. a modulated level of FAD covalent attachment to enzymes,
f. modulated or modified N-linked glycosylation, vesicle transport, protein degradation, lipid degradation, carbohydrate degradation, or heat shock proteins,
g. modulated reactive oxygen species pathway activity, and
h. modulated cellular protein sorting.

25. The cell of claim 1, wherein the cell also expresses a prenyl transferase and produces CBGA or CBGVA by prenylating OA or DVA with GPP.

26. A method of producing CBDA, THCA, CBCA, CBDVA, THCVA, CBCVA, or analogs thereof, comprising contacting the cell of claim 1 with a carbon source and, optionally, hexanoic or butyric acid and suitable conditions to produce CBDA, THCA, CBCA, CBDVA, THCVA, CBCVA or analogs thereof.


SEQUENCE LISTING (126 sequences)

SEQ ID NO: 1 - protein, 516 aa, synthetic construct
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 2 - protein, 516 aa, synthetic construct
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHHH

SEQ ID NO: 3 - protein, 516 aa, synthetic construct
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGVGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 4 - protein, 516 aa, synthetic construct
NPRENFLKCFSQYIPNNATNLKLVYTQNNPLYMSVLNSTIQNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVGVGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 5 - protein, 516 aa, synthetic construct
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMRSIKIDVHSQTAWVEAGATLGEVYYWINEKNENLSFPGGYCPTVGVGGHFSGGGYGALMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFNRLVKVKTKVDPNNFFRNEQSIPPLPPHRH

SEQ ID NO: 6 - protein, 516 aa, synthetic construct
NPRENFLKCFSQYIPNNVANLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 7 - protein, 516 aa, synthetic construct
NPRENFLKCFSQYIPNNATNLKLVYTQNNPLYMSVLNSTIHNLRFTSDTTPKPLVIVTPSNVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 8 - protein, 516 aa, synthetic construct
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFTTPYVSKNPRLAYLNYRDLDIGINDPKNPNNYTQARIWGEKYFGKNFDRLVKVKTLVDPNNFFRNEQSIPPLPRHRH

SEQ ID NO: 9 - protein, 516 aa, synthetic construct
NPRENFLKCFSQYIPNNVANLKLVYTQNNPLYMSVLNSTIHNLRFTSDTTPKPLVIVTPSNVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 10 - protein, 516 aa, synthetic construct
NPRENFLKCFSQYIPNNVANLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFTTPYVSKNPRLAYLNYRDLDIGINDPKNPNNYTQARIWGEKYFGKNFDRLVKVKTLVDPNNFFRNEQSIPPLPRHRH

SEQ ID NO: 11 - protein, 516 aa, synthetic construct
NPRENFLKCFSQYIPNNATNLKLVYTQNNPLYMSVLNSTIHNLRFTSDTTPKPLVIVTPSNVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFTTPYVSKNPRLAYLNYRDLDIGINDPKNPNNYTQARIWGEKYFGKNFDRLVKVKTLVDPNNFFRNEQSIPPLPRHRH

SEQ ID NO: 12 - protein, 516 aa, synthetic construct
NPRENFLKCFSQYIPNNVANLKLVYTQNNPLYMSVLNSTIHNLRFTSDTTPKPLVIVTPSNVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFTTPYVSKNPRLAYLNYRDLDIGINDPKNPNNYTQARIWGEKYFGKNFDRLVKVKTLVDPNNFFRNEQSIPPLPRHRH

SEQ ID NO: 13 - protein, 516 aa, synthetic construct
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDCKNPNNYTQARIWGEKYFGKNFDRLVKVKTLVDPNNFFCNEQSIPPLPRHRH

SEQ ID NO: 14 - protein, 516 aa, synthetic construct
NPRENFLKCFSQYIPNNATNLKLVYTQNNPLYMSVLNSTIHNLRFTSDTTPKPLVIVTPSHVSHIQGTILCSKKVGLQIRTRSGGHDAEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 15 - protein, 516 aa, synthetic construct
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYVLYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 16 - protein, 516 aa, synthetic construct
NPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 17 - protein, 516 aa, synthetic construct
NPRENFLKCFSQYIPNNATNLKLVYTQNNPLYMSVLNSTIHNLRFSSDTTPKPLVIVTPSHVSHIQGTILCSKKVGLQIRTRSGGHDSEGMSYISQVPFVIVDLRNMRSIKIDVHSQTAWVEAGATLGEVYYWVNEKNESLSLAAGYCPTVCAGGHFGGGGYGPLMRSYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSARQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSQNPRLAYLNYRDLDIGINDPKNPNNYTQARIWGEKYFGKNFDRLVKVKTLVDPNNFFRNEQSIPPLPRHRH

SEQ ID NO: 18 - protein, 516 aa, synthetic construct
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNSRLAYLNYRDLDIGINDPKNPNNYTQARIWGEKYFGKNFDRLVKVKTLVDPNNFFRNEQSIPPLPRHRH

SEQ ID NO: 19 - protein, 516 aa, synthetic construct
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITKNITDNQGKNKTAIHTYFSSVFLGGVDSLVDLMNKSFPELGIKKTDCRQLSWIDTIIFYSGVVNFDTDNFNKEILLDRSAGQNGAFKIKLDYVKKPIPESVFVQILEKLYEEDIGAGMYALYPYGGIMEEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPPHHH

SEQ ID NO: 20 - protein, 517 aa, synthetic construct
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 21 - protein, 517 aa, synthetic construct
NPRENFLKCFSKHIPNNATNPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 22 - protein, 517 aa, synthetic construct
NPRENFLKCFSKHIPNNVANPKLVYTQHDQLYMSILNSTIQNLRFISDTTPKPLVIVTPSQNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 23 - protein, 517 aa, synthetic construct
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFMTPYVSQNPRLAYLNYRDLDLGKTNHASPNNYTQARIWGEKYFGKNFNRLVKVKTKVDPNNFFRNEQSIPPLPPHHH

SEQ ID NO: 24 - protein, 517 aa, synthetic construct
NPRENFLKCFSKHIPNNATNPKLVYTQHDQLYMSILNSTIQNLRFISDTTPKPLVIVTPSQNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 25 - protein, 517 aa, synthetic construct
NPRENFLKCFSKHIPNNATNPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFMTPYVSQNPRLAYLNYRDLDLGKTNHASPNNYTQARIWGEKYFGKNFNRLVKVKTKVDPNNFFRNEQSIPPLPPHHH

SEQ ID NO: 26 - protein, 517 aa, synthetic construct
NPRENFLKCFSKHIPNNVANPKLVYTQHDQLYMSILNSTIQNLRFISDTTPKPLVIVTPSQNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFMTPYVSQNPRLAYLNYRDLDLGKTNHASPNNYTQARIWGEKYFGKNFNRLVKVKTKVDPNNFFRNEQSIPPLPPHHH

SEQ ID NO: 27 - protein, 517 aa, synthetic construct
NPRENFLKCFSKHIPNNATNPKLVYTQHDQLYMSILNSTIQNLRFISDTTPKPLVIVTPSQNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFMTPYVSQNPRLAYLNYRDLDLGKTNHASPNNYTQARIWGEKYFGKNFNRLVKVKTKVDPNNFFRNEQSIPPLPPHHH

SEQ ID NO: 28 - protein, 517 aa, synthetic construct
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNQYTQARIWGEKYFGKNFNRLVKVKTKVDPNNFFRNEQSIPPLPPHHH

SEQ ID NO: 29 - protein, 517 aa, synthetic construct
NPRENFLKCFSKHIPNNVANPKLVYTQHDQLYMSILNSTIQNLRFISDTTPKPLVIVTPSQNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNQYTQARIWGEKYFGKNFNRLVKVKTKVDPNNFFRNEQSIPPLPPHHH

SEQ ID NO: 30 - protein, 517 aa, synthetic construct
NPRENFLKCFSKHIPNNVANPKLVYTQHDQLYMSILNSTIHNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 31 - protein, 517 aa, synthetic construct
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQLPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVEAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 32 - protein, 517 aa, synthetic construct
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGQNGAFKIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 33 - protein, 517 aa, synthetic construct
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLTTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTATWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 34 - protein, 517 aa, synthetic construct
NPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAACKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 35 - protein, 517 aa, synthetic construct
NPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVVVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 36 - protein, 517 aa, synthetic construct
NPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDSKELSWIDTTIFYSGVVNYNTANFKKEILLDGSAGKKTAFSIKLDYVKKLIPETAMVKILEKLYEEEVGVGMYVLYPYGGIMDEISESAIPFPHRVGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 37 - protein, 517 aa, synthetic construct
NPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYDGIMDEISESAIPFPHRAGIMYELWYTATWEKQEDNEKHINWVRSVYNFTTPCVSQNPRLAYLNYRDLDLGKSNPESPNNYTQARIWGEKYFGKNFNRLVKVKTKADPNNFFRNEQSIPPLPPRHH

SEQ ID NO: 38 - protein, 506 aa, synthetic construct
HENFLQCLFLHFPNSNSISKVIYTPYNSSYESIWLSEIRMDRFKPPAPITKPLAIVKPLEDSHIQTSIYCSKRHDVQIRIRSGGHDYEGLSYISYEAPFIIVDLENFQSITIDVENSTAWVQAGATIGQLYYTIAQKSPTLGFSAGICPTVGVGGHFSGGGYGMMSRNYGISADNIIDARLIDVNGRILDRKSMGEDLFWAIRGGGSASFGVIIAWHIKLLQVPETVTVFTVNRTLEQNATKIVHRWQSVSAKIDEKLALRLYLRRANSSQDGKETGQASFVTLYLGGVDTLLPLMEQSFPELGLARSDCIEMSWIDSILYFASLSGEPLDVLLNRTPSPYGLYYKGKLDYVTEPISETGLESIWKRYLEYPEEMNELELSPYGGRLSEISKSATPFPNRAGTLYSIHYAVYWAEAGKSALNRHISGIRRFYSFMTPYVTKNPRSTYVNCRDLDLGVNNKGNTTSYEQASIWGLKYYGINFKRLVHVKTKVDPGNFFRNEQSIPPL

SEQ ID NO: 39 - protein, 505 aa, synthetic construct
NLASHNFLKCFHSQTNLSVDSNSSSSSNVIFTTNGSSYTSVLQSSAQNLRFATASTPKPQLIVRPLHESHIQAAVICSRKNSINIRIRSGGHDYEGLSYVSNIPFVIIDLANLRSIKIDVENKTAWVESGATLGELYHAIAEESNTYGFPAGSCPTMGVGGHLGGGGFGTIFRKYGLAADNVIDARIVDVNGRIFDRKSTGEELFWAIRGGGGASFGIIFAWKINLVPVPSIVTVFDIGKTLEPAGATKLLQKWQLVADKLSDTLFLDVVIAGNSNNTIAASFRALFLGVSEELLSTMEESFPELGVERKDCTEMSWIQSVLYFAGFSTRESYDVLLNRTNPYKSSFKAKSDYVKEPISEKGLQGLWKILGEEESGYMIWTPYGGRMSEISEFEIPFPHRSGNLYLIQYMVTWEAEEETEEHVNWITRLYEYITPFVSKNPRSAYLNYRDLDLGINMNENESYEQASVWGLKYFNSNFKRLAQIKAEVDPSDFFWYEQSIPLFLS

SEQ ID NO: 40 - protein, 505 aa, synthetic construct
DSSYQTFLQCLKTKSPPQSPEISSILYDPTNSSFQTVLQSYVRNRRFNRSTTPKPLLIVTPKTETHVSYAVLCAQAINIQLKIRSGGHDYDGISYVSDQPFIILDMFNLRSISIDLPTQTAWVQAGAQLGELYYQISTKSKTLGFPAGVCPTVGVGGHLSGGGYGNMLRKYGLSVDHVTDARIVDVKGRILDRKAMGEDLFWAIRGGGGASFGVVLAYKIGLVPVPETVTVFRVERTLAQNATEVVYRWQYVADKIDNGLFIRLLLQPVTVNKTRTIRASFIGEFLGDCVKLMEVVGNGFPELGFKKEDCMEMSWIGSVLYWANFDNDTKPEALLSRTPDSVNFGKRKSDYVQNPISKDGLEWIWKKMIEVGKTGFVFNPYGGRMSEIPAGETPFPHRAGNIFKMQYSVNWNEEGVDADKDYINQIRRLYSYMTPFVSKSPRGAYLNYRDLDIGTSANGKNSYEEGKVYGEKYFMGNYARLVKVKTMVDPGNFFRNEQSIPPFLS

SEQ ID NO: 41 - protein, 521 aa, synthetic construct
RIHDYQYPKTSFLQCLSHHSSSNSSNNDLTKVVYTTTNSSYFSVLNFTIINPRFSSPSTPKPLFIITPLYESQVQAAVICARKHGVQIRVRSGGHDYEGLSYVSEIPFIVIDMINFRSITVDVERRTGWVQTGATLGELYYEINKKSKTLAFPGGACPTIGVGGHITGGGYGAIFRKYGLAADNVIDAQLVDVEGRVLDRETMGEELFWAIRGGGGASFGVILAWKVLLVRVPETVTVFSINRNLEHNLTKLVHRWQYIAHKLPKKLLLAVRFQTVNSTKKGIEMKKLQATFISVFLGRVDGLLYLMEKRFPELGLVREDCTQMSWIQSALFVAGLPSEQSPEILLDRTPQSRLSFKAKSDYVKEPIPEKGLEGIWERLYEEEVGSGIVIMSPYGGKMSEIPESELPFGHRGGNMYKIQYLIYWEEEENSGIVVEKHISWIRRLYNYMTPFVSKNPRSAYINYRDLDIGRNSNKGTATYAQASIWGVKYFGQNNFNRLVHVKTMVDPTNFFRNEQSIPPLP

SEQ ID NO: 42 - protein, 513 aa, synthetic construct
NPHNNFLQCFSKHISNNNTSLAKLIHTPNDFSYISLLNSTIQNLRFTSPTPKPLVIITPSNTSHVQACVLCSKKYGLQIRTRSGGHDFEGVSYVSKVPFVILDMRNLRSITIDVDNKTAWVESGATLGELYYRIAEKNENLSFPGGYCHSVGVGGHFSGGGYGALMRKYGLAADNVIDAHLVNADGEFVDRKSMGEDLFWAIRGGGGASFGIVLAWKIRLVPVPSKVTILSVSKNLPINETVKIYNKWQNIAHKFDQDLLMVVRFLTVNSTDEHGKNMTTIQATFFSIFLGRVDKFLSLMQTNFPELGVVRKDCFETSWIEMIFFFNEFSSEDKLEVLLDPTNVVKGYFKGKLDYVRKPISEIVMVKLLEKLYEEDVGLAYIQMYPYGGKMSEIPESAIPFPHRAGVMYKILYWSQWEKEEESERHMNWVRSVYNYMTPYVSKNPRASYINYRDLDLGTNNEKGPISYEQASIWGKKYFNKNFKKLVQVKTKVDPTNFFRNEQSIPPSFPRLL

SEQ ID NO: 43 - protein, 515 aa, synthetic construct
NPHDNFLQCFSKHINNNNNKSIVKLIHTPNDPSYISVLNSTIQNLRFASPSTPKPLVIITPSNTSHVQACVLCSKKYGLQIRTRSGGHDFEGASYVSKVPFVILDMRNLRSITVDVDTKTAWVEAGATIGELYYRIAEKNGNLSFPAGYCRTVGVGGHFSGGGYGALLRKYGLAADNIIDAHLVNADGEFLDRKSMGEDLFWAIRGGGGASFGIILAWKIRLVAVPSKVTMFSVSKNLEMNETVKIYNKWQNTAYKFDKDLLLFVSFMTINSTDSQGKYKTTIQASFSSIFLGRVESLLILMQKKFPELGIERKDCLEKSWIETVVYFDGFSSGDTPESLLNTTFQQNVFFKVKLDYVKKPVPEVVMVKLLEKLYEEDVGVGFLMMYPYGGKMDEISESAIPFPHRAGFMYKILYLSAWEKEGESEKHMNWVRSAYNFMSPYVSQNPRATYLNYRDLDLGTNNEKGPISYSQASVWGKKYFGMNFKRLVNVKTKVDPSNFFRNEQSIPPLLSRRL

SEQ ID NO: 44 - protein, 506 aa, synthetic construct
SCLCSTAAQACSILASTLPAEAFVAPNSTGYQKVQTSYWSETAGDLRPACITRPGSTEQVASIVKVLSTCGSDVRFAIKSGGHGSWPGWSSTEGGVLISLDLIDGVQNVADKGYAVVGSGARWVDVYKSLEPQGVTVIGGRFASIGVGGLIVGGGISYFTGLHGMACDNVLNYEVVLADGTIANVNRTSKPDLFRALKGGGNQFGIVTRFSLKTYKQTTQVWAGILTFSIDKASQVFNATQAFIDRYDPLSHMYLSMGGGSNPQLSFINAYVFYDASSPPAGEANPFAAFYAAKPIADTTSTQNYSALIASNNNGNTPQQRWLIGGHTFPNLVAPYGADLYLKQWQAFRDMANATAAGDQALLFSMALVPVPARAMRAGENVTGVKNVLGLEPDAGDAVWIDYTLAWLEAKDDGKMFEFVERISKAGDKLATSFAPGVASTNAMQGAVTAKEAGFYRTNPRYMNYALLDQPVQSSYGKENEQFLQRVQKRYDPDGLWRKNGGFLWG

SEQ ID NO: 45 - protein, 565 aa, synthetic construct
KPISQNGANDGLRALTKRTVSPDGSCGGSNTYTCPTGQCCSQYGYCGTTTAYCGTGCQSGFGTCSGSTPPTSTSTSTPSSATPTSTTLQDCLGAKNVPVSFTNSAGFSALAQPYNLRLAYTPAVIVLPTTTQHIIDAVLCAGKSNVKVQAKGGGHSYASFSSGGQNGAMVVDLESFQQITVGAGGVAQIGGGVRLGDMALGIFNQSQRALPHGTCPGVGVGGHASHGGFGLDSRLWGLTLDTIVGLDVVLANGSFIHATSTAYPDIYYALRGAADSFGIITTFYLQTLPAPASVVNWQYSIPGMFASAATSTAVFAHIQTFAQNASVVDRKLGLGIYLDGSGFSVSGTYIGDLATFNSKIAPELLRGLPTPSSSSVKAVDWIQSLTLLANGQPLQQPTSRTSYTPHDDFFAKSLVVPSSAPLTTAALTSYFTYIIQNGVNAPNPWYSIINLYGGPDSQINVPSPSSSAYSDRSALWVLQHYGYTGNTGSPYPPASLTFVNGLNTAITSAMPSTTFGGYLNYVDPSLTAAQAHTLYYGTTTYAKLLSIKNVVDPGKVFSNPQSIGN

SEQ ID NO: 46 - protein, 485 aa, synthetic construct
SKINPINPFFENDIGCEKVKYPSEILAPIPYLKECLTTIDGLVVYPDDPYYYHSIKVMNCRNSYFPVVVIYVTTISDIQKSIDCANTLNISVTARSGGHSYEEYGNGGKSGAMVIDVQELNQIDINFETKTAVIGTGNRLGTIYYKLYKDGFLLPAGYCLNVGIGGHATGGGYGMVGRKYGMASDNIVSAEMVTANGTFLSPINSKHHSDLFFAIRGAGNAGYGIITSLTFKIYPIWPIVTTINIAYFSDQIELAFNILAKVAKNLSENLNAYIFLIPKINNNSNTLLINATYLGSEEEARNAAKELIELSNPAYIKFKEMPWWKAVKAQSPPFNRQPFKVTSYTIPPQGFSREAIRFLVNFINNMECMSGVLFDLYASDALSRISVNSSAFIHRNILFVMELETMLDGHSKNVQNKCVKKHLQFSQEFQAKYTSYFSYQDYIDKDLPDWLTRYYGSHVPRLIETKKKYDPDNLFNWKQSIPTEL

SEQ ID NO: 47 - protein, 474 aa, synthetic construct
SPFYGPPDALTSCLATASVPYAAKNSSQWIQDTKPYNLRLAYVPEAVAIPTTIKHIQDAVKCARQHGVRVSAKSGGHSYGSFGFGGEDGHLVVVMDAMDKVTLNKDMSCTIEAGARLGHVANELFNNAYRRALPHGTCPGVGITGHALHGGYGMSSRTYGLTLDRLVAATVVMADGSIKHSSVWDTPNFHWALRGAGSSFGIVAELDFMTFAAPEVLTSFSIDLDWTEEEAVEGILAFQEFGVNAPKELNMQIFMGPRGQTIQGLYHGSLEGLNTALRPLLGEINAQVSKTKTMNWIESVGHFSDGQSIVQRRPYDRHSTFYTTSLMTHALTRHQVESLANALFTNARDPSARKSWYLLLDLFSGPNSAISEKAPSDTAFPHRDKLLLYQFSDGGTNGVYPPEGFDLLRRFRESVTSSMVDGEWGMYANYLDTQLDGDTAARLYYGGNLERLRVLKGEFDPDDVFWNPQGIRPA

SEQ ID NO: 48 - protein, 483 aa, synthetic construct
YVSLTEAPPTSLIGCLQSIKGNKKSYLSIPSSPTYNNDRLGYNRNFVHKPSAIFHPASEADAVAAVVCAAAHNVSIAPRSGGHSYEGYSGGGKTASLVIDLNLFLQFAIDHDTGIATVGAGNRLGTLCTKLWKAGEYLIPAGSCPGVGIGGHALGGGLGIAGRKYGMLSDNIVGLTMVAANGNILEVNARSRPDLFWALRGAGGGSFGLVTEFRIQAYKAPARVTTMEVKYPWSVHQNVINAFGIWSKSVVDDFTGMLYIGQGKVRVLGTFLGSQEEAQTALKSLFTLTGLPQQTTFNESTWYQAAARWAEMEGANLEDPYTEHALAFRFRSLLYRQPISDSELAIISKHINNPPNNPDGQVTISVSLEIWGGKIDYPSSASAFDNHRGILYSIRYGVEWNVSSPDSDSICTECMEWNDHFAKELQAAYSSGQVLEAYQNYIERDIPNGLQAYYGDNLSRLIEIKKSVDPGNIFVFPQAIPLS

SEQ ID NO: 49 - protein, 485 aa, synthetic construct
QYTSLREQLASNGIRAVFADDVEYGNATRGYNMRYTPSPIAVTFPTNTSQVSLIVKAGAKEGMRVVAKSGGHSYIANSLGSDSDPPALVVDLGAFKNITYDGETQTAVIEPGNRLGEVALALNEYGRAIPHGRCAWVGFGGHSGFGGWGFSSRMWGLTLDNVLSATVVLANGTVVTASEDENTELFWAIRGSSSSFGIVTALTMRTHPVPPSGTFFEYIFDLDIATASKAFSDFQKYALSPSLPNTFGGELFVLKGEEKGRVYIVFLGSFYGDTERYAQVVTPFLDTLPPWRNDSIVLNGTWIDVLAAAAVGNLTVQGEMPRDTFYVKSLMTPQDVLLTEEAMHGMMRYLGEEGFDSDAFWHVEIEIYGGVQSAINSVPLNTTAFGHRDTLFTFQPYVSTADFLPPWDDAIFGFVDGIMSSITDNMPSDWEYGAYANYIDPRLPDWQHRYYGANYERLQKLKGEVDGDDVFKFITSIEVPEEGKR

SEQ ID NO: 50 - protein, 509 aa, synthetic construct
RNDVTETDIPAFAAATITADDPRYPSLVLGTNHRFPGRPDYVRVVTTTAQVAAAVTEAVAAGRRISVRSGGHGFEDLATNGVEVLLDMSEMTEVRYDEERRAFAIEAGATLGHVYRVLFKGWGVTIPAGECPEVGVGGHIVGGGYGPLSRSLGAVVDYLYAVEVVVVGEDGVARAVVATREDGDPHRDLWWAHTGGGGGTFGVVTRYWMRAPGADSGSDDPAELLPKAPAAWRSGMAVWSWETMTEESFAALQANFGAWFERNSDPDSPYAALAGFFHGTHRSGFGLTVGAHMDDATADAERLMTSFFEEITAGVDARPLFVDQQVRPWLYMSSYPGWGDPGDPFTRRIKIKAAYLRKGYSATQTAALYRHLTAGEASPVQLILIGYGGRVNAVAPDATAAAQRDSVLKAAYMAVWGAEADDETNIRRVREMYREVYAETGGVPVPNDANDGSYVNYADTDLADPEWNTSGVPWHTLYFKDNYPRLQRVKKRYDPRNVFRHALSVGLPG

SEQ ID NO: 51 - protein, 504 aa, synthetic construct
ADEASCPPAPGPAMVGRGDPRYRSLASRGYNRRFVGGPEHVWVVGTTAHVVRAVQQAVDSGRRITVRSGGHGFENFVADPAVEVVVDMSAMTAVSYDPDRRAFAVEAGALLGEVYRRLYLGWGVTIPAGWCADVGIGGHVLGGGFGPLSRLMGLSVDHLYAVEVVVVDRAGKARRVVATREPSDPNRDLWWAHTGGGGGSFGIVTRYWFRTPGADGDDPSALLPAPPSGVLSFSVTWDWQKLDRTSFARLVRNHCAWAERHSAPHSPYLALYSELALTRRPSGTVLMIGQVAADSGAERMLDEHLAAINRGVPVQPVRTVRNHSWLAAALAGSPGDPGPVYRLKVKSGYLRRRFTDRQIDTLHHHLTRSDYDHPGGSVSLYTHGGKVNTVAGDATATSHRDASIKMFYVNGWEDPRDDARHIAWLRELYRDLYADTGGVPAAADGAFINYPDTDLADPAWNTGAPWHTLYFGENYRRLQRIKAKWDPRDVFHHALSVRPALRRG

SEQ ID NO: 52 - protein, 482 aa, synthetic construct
RPPYATGDSPVPAAEVLPGDPRYPALVTGFNQRFTGTPAAVVVVTTQDQCVAAVRAAVAAGRRVTVRAGGHCYEGFVTDNPGGVVLDVSGMRAVTRTPDGGIRLESGCTNWDVYELLYKSHGVTLPAGSCYSVGLGGHVVGGGYGLMSREHGLTVDHLSALDVVVVDADRSVRVVTARRGDPLTGDLFWAHTGGGGGTFGVVVAYTFRDLPAPPPTVQLATTTWQWSALDAASFATLLRNYGGWLAANSGPDSPGRRLFGLLKLSHVSAGTVTLLTQASGEDPALLDGFLGALDDGMPAAALPTTTRRTMPWLQATQTLNGSGPNQRGKYKSAYMRRPFPDRHVAALHAALTDASYRNPQALVQVDTYGGRINAVASGDTAVSQRSSIMKLQYQTYWTDPAEDELHLGWIRRCYASVYADTGGEPARDDVTDGCYVNYPDVDLRDWPTLYFGPNYRRLQEVKGRWDPTDQFHHAQSVRPPGR

SEQ ID NO: 53 - protein, 526 aa, synthetic construct
HSHEDFLQCLSHHFSNSTTFAKLIYTPNDPSYISVLNSTIRNPRFSSLSTPKPLLIITPSNSSHVQASVYCSKKHGLQIRTRSGGHDYEGLSYVSEVPFVIIDLRNLNSVIVDVHEKTAWVEAGATIGEVYYRIAEKSGNLSFPAGVCHTVGVGGHFSGGGYGFLMRKYGLAADHIIDAQLVNAEGKFLDRESMGEDLFWAIRGGGAASFGIVLAWKIRLVPVPSTVTISIVDRNLEQNETLKLVNRWQSVADKFDEDLLITVGFQTVNSTDREGNNKTIIQATFVSLFLGKVDGLLSLMEKSFPELGLKRGDCIELSWIEVVHYFAGFPLRGELEDLLNRTPQEGLSPFKTKLDYVTKPISEKVLETVLQRLYEEEVGKGLIQFFPYGGKMSEIPESEIPFPHRAGNLYKIFYRIEWDEGERDVKAVERHLDWVRSVYNYMTPYVSKNPRGAYLNYRDLDLGTNNDKGPTSYAQASIWGKKYFGSNFKRLVHVKTKVDPKNFFRNEQSIPPVPSPGVNKNNNYTP

SEQ ID NO: 54 - protein, 526 aa, synthetic construct
HSHEDFLQCLSHNFSNSTTLAKLIYTPNNPSYISILNSTIQNPRITSPSSLKPLLIITPSNASHVQASVYCSRKHGLQIRTRSGGHDLEGLSYVSEVPFVIIDLRNLISINVDVHEKTAWVEAGATIGEVYYRIAEKSGNLGFPAGVCHTVGVGGHFSGGGYGNLMRKYGLAADNIIDAQLVNADGELLDRESMGEDLFWAIRGGGAASFGIVLAWKIHLVPVPSTVTMFTVDRNLEQNETLKIVNRWQYIADKFDEDLLIFVRFLTVNSTDQEGNNKTTIQASFFSLFLGGVDNLLSLMAKRFPELGLKREDCTEMSWIESVHYSAGLPSGKLEGLLNRTPLEVPILPFKVKLDYVKKPIPEKVLETMLDRLYEEEVGKGLIQLFPYGGKMSEIPESEIPFSHRAGNLYKILYYVKWDEGENDSKAVERHLNWVRSVYNYMTPYVSKNPRGAYLNYRDLDLGTNNDKGPISYAQASIWGKKYFGSNFKRLVHVKTKVDPKNFFRNEQSIPPLPLPGAKKNNNNGP

SEQ ID NO: 55 - protein, 524 aa, synthetic construct
LSDTTPEKFYQCICKNSDFSDPFSKAFFTPNTDSFNTVLNSTAQNLRCLMPTVPKPVLIFTPMAESHVQAAVICAKQLNLQLRVRSGGHDYEGLSYISVMDSPFVIVDLSKLREIDVNIEENSAWAQAGATVGEVYYRISEKSKTHGFPAGLCTSLGIGGHITGGAYGSMMRKFGLGADNVIDARIVNANGTILDRLSMGEDLFWAIRGGGGASFGIILSWKLKLVTVPSVVTVFTVPKTLEDGATKILYKWQQVADKIDDDLFMRVVINVVDKKDKKGERTVQTAYNSLFLGNVDRLLQIMNESFPELGLTQKDCTEMSWIESILYIAGFPTKTPPEILLQGKPTFKNYFKAKSDFIKEPIPETGLDGIWKRLLEEDSPLMIWNPYGGMMANISESETPFPHRKGVIFKIQYLTLWTEPDQELATKHVDWIRKLYSYMTSYASMFPREAYVNYRDLDLGMNKNGNSNSSFVQATVWGNKYFNNNFNRLVEIKTKVDPENFFKFEQSIPTLPLTKKRKGKKINH

SEQ ID NO: 56 - protein, 507 aa, synthetic construct
AVTKPGIGEFLGCLRSWPSPESPITDDIFTADNTTTFLSSYLAYTKNTRFESPNYQTLLAIVTAKNLSHIQATVVCAKSNGVQIRIRSGGHDYEGLSYISSVPFVILDLFNLRSITVDVPSKQAWVEAGATLGELYTKISDASETLAFPAGVCSTVGAGGHISGGGYGNLMRKYGLTVDNVVDALIVDVNGKLLNRSTMGEDLFWAIRGGGGGSFGVILSWKINLVDVPEILTVFRVNKTLEQGGIDVLYKWQLVSSKLLDGLFLRALSQVVNRTITVEFYAQFLGRADELVAIVNQSLPELGLKRQDCLEMSWLNTTLFWEDLPVGTPTSVLLDRPSKPEKFFKSKSDYVKKPIPKEGIKKLWEALLEFNNNNIVYMQWNPYGGVMDTVPAAATPFPHRKGNLFEVQYYTSWLDANATMGSLDMMKKLYEVAEPYVSSNPREAFLNYRDIDIGSNPSNETNVEEAEIYGSKYFVGNLKRLMEVKAKYDPENFFKNEQSIPPARVKQ

SEQ ID NO: 57 - protein, 503 aa, synthetic construct
AVESFLQCFTSDIKNNSSKIIITKSSSAYFSAVQSSIQNLRFVTSATSRPEAIITPLHDSHVQAAVICSKKKRILIRIRNGGHDYEGQSYVARAPFVIIDLFNLRSIDVDIENESAWVESGATLGELYYRIAEKGKAYGFPAGSCPTVGVGGHISGGGFGPLFRKYGLAADNVLDAKIVDVNGRILDRKSMGEELFWAIRGGGGSSFGVILAWKLRLVPVPPSVTVFKISRTTEQGATKLLSKWQNIADKLHEDLFLHSVIGVGNKAGTNGDKTIIIEFSSLFLGPVEKLLPLMQDNFPELSVDRSDCTEMSWIESVLYFASISRNESEALLSRTQQSKSFFKAKSDYVNEPISEAGLEGLWQTLIEVKAYLILTPYGGKMSEISDSETPFPHRSGNLFKILYMVTWDDYRENEIGESLMSKLYDYMAPYVSKSPRAAYLNYKDLDLGRNNDVHTSYAQASIWGLKYFKNNFRRLVHVKTLVDPGNFFRDEQSIPSVRSFRKK

SEQ ID NO: 58 - protein, 527 aa, synthetic construct
EHCLPGDGTGCWPSQAEVKALVDSLSSEAEVYLPTGPNADMYMSYATDTPGNTLKPSSPGLIVFPMNEGDVRKVVTFAAKFRIRLVGKCSGHDQLQRSNGYGVIQLVTTKGFRNISYNETDHTITFGAGNAHKDVYAFLADRKRFAVGGTWAYVCPAGCLSNGCYGFFTKEFGSGADNVVGLRFMLFNGTTLNLDAAGPHRDLFWAILGGGASSYGVLLGITAKTYEAPDQFIIMKVFLSIWPTGLTKDQAPSVFTRFLTNLTWYQALPPHISWQLDNNPAEQRVLLQISYTGRTTVDVATEELSGLVAEWTEAGYFQEHGVEGYSDVADFAVKNSGAGPPIRRVSQSIYVDANQDALDKVAALWETLYSDAYMSPVGSDPFDGAFNFWAQYRYGPGSYAGKAPHVARGMRTAMMEIAHENVWMRQADDSFRAAISNDVKELMEPLGNTSYQNHCSTWAEYNVYVDYKTRFYQNWEGLTAVKKKYDPCNLYVVQYGPGWDLPKATCGLSTTSTKRPPPKRPPPRRHA

SEQ ID NO: 59 - protein, 517 aa, synthetic construct
NPQENFLKCFSQYIPTNVTNAKLVYTQHDQFYMSILNSTIQNLRFTSDTTPKPLVIITPLNVSHIQGTILCSKKVGLQIRTRSGGHDAEGMSYISQVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKTDCKQLSWIDTIIFYSGVVNYNTTYFKKEILLDRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLPLRHH

SEQ ID NO: 60 - protein, 517 aa, synthetic construct
NPQENFLKCLSQYIPTNVTNAKLVYTQHDQFYMSILNSTVQNLRFTSDTTPKPLVITTPLNVSHIQGTILCSKKVGLQIRTRSGGHDAEGMSYISQVPFVIVDLRNMHSVKIDVHSQTAWVESGATLGEVYYWINENNENLSFPAGYCPTVGTGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKTDCKQLSWIDTIIFYSGVVNYNTTNFKKEILLDRSGGRKAAFSIKLDYVKKPIPETAMVTILEKLYEEDVGVGMFVFYPYGGIMDEISESAIPFPHRAGITYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLPLRHH

SEQ ID NO: 61 - protein, 514 aa, synthetic construct
NPQENFLKCLSQYIPTNVTNAKLVYTQHDQFYMSILNSTVQNLRFTSDTTPKPLVITTPLNVSHIQGTILCSKKVGLQIRTRSGGHDAEGMSYISQVPFVIVDLRNMHSVKIDVHSQTAWVESGATLGEVYYWINENNENLSFPAGYCPTVGTGGHFSGGGYGALMRNYGLAADNDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKTDCKQLSWIDTIIFYSGWNYNTTNFKKEILLDRSGGRKAAFSIKLDYVKKPIPETAMVTILEKLYEEDVGVGMFVFYPYGGIMDEISESAIPFPHRAGITYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLPLRHH

SEQ ID NO: 62 - protein, 517 aa, synthetic construct
NPQENFLKCFSQYIPTNVTNAKLVYTQHDQFYMSILNSTIQNLRFTSETTPKPLVIITPLNVSHIQGTILCSKKVGLQIRTRSGGHDAEGMSYISEVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFAELGIKKTDCKQLSWIDTIIFYSGVVNYNTTNFKKEILLDRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLPLRHH

SEQ ID NO: 63 - protein, 517 aa, synthetic construct
NPQENFLKCFSQYIPTNVTNAKLVYTQHDQFYMSILNSTIQNLRFTSETTPKPLVIITPLNVSHIQGTILCSKKVGLQIRTRSGGHDAEGMSYISQVPFVIVDLRNMHSVKIDVHSQTAWVEAGATLGEVYYWINENNENLSFPAGYCPTVGAGGHFSGGGYGALMRNYGLAADNIIDAHLVNVDGKVLDRKSMGEDLFWAIRGGGGENFGIIAAWKIRLVAVPSMSTIFSVKKNMEIHELVKLVNKWQNIAYMYEKELLLFTHFITRNITDNQGKNKTTIHSYFSSIFHGGVDSLVDLMNKSFPELGIKKTDCKQLSWIDTIIFYSGVVNYNTTNFKKEILLDRSGGRKAAFSIKLDYVKKPIPETAMVTILEKLYEEDVGVGMFVFYPYGGIMDEISESAIPFPHRAGIMYEIWYIASWEKQEDNEKHINWIRNVYNFTTPYVSQNPRMAYLNYRDLDLGKTNFESPNNYTQARIWGEKYFGKNFNRLVKVKTKVDPDNFFRNEQSIPPLPLRHH

SEQ ID NO: 64 - protein, 472 aa, synthetic construct
GFPAANLSSVLSSPLLGLSPATEIFYPSDADWTNETQRWTLHDSPTYLAAVKPATERDAQKLVAFAAKSNISFLATGGAHGFTTSYGTLQNGIMLDLSNFRNVSVDAAANTMRIGAAVRFGDVYEPLYNAGKEITTGTSSCVGMLGATLGGGLGRYNGIHGMILDALISVRLIIATGEIITASTTENADLFWAVRGAGFNYGIVIEATYRITDLTSQYVINADFAFPMNASTALLTYLKSFETDMPEKLSLITYVTYVAAYGGLSFTFNAVYAGPKDELDQLLSPLLRDATPIRENITVIPWKDLTYTAFFAAEPSMAACTKGLYRNVYGGSVKAYDISSFNTFFTEMQDFYLEYPDAQSSVFFIEHFPKAKALSVPDNATAYPYRDITAHLLWNFGYPAGDLEKPVNAFGVKARADLQASSGFLPQQIYVNYAHGDEAPEILYSARNLPRLRNLKEVWDPENVFRFNEPVV

SEQ ID NO: 65 - protein, 482 aa, synthetic construct
LSPSFEHVVDRGSNIFSELDAQLSPNASIILPADPLFQTAGARWQAYSRPTYSAVVEVAVEEDVQKTVRFANKYSIPFLAVDGAHGFISTLGRMQNGIAISFSKMKGITVSRNGKFADLNPGLTNGDIVRTLWAQNKYTVTGNCMCTGIMGIMLGGGHGYLQGLYGLAADQILEARVVLADGSCVTASPESNPDLFWALRGAGHNFGIVTGLKYKVYESLSLWTEIQMIFKGDKVEEFFEVANNLTQEENHPAKLIHWSNHIRRPDIDPDNAILNTYFIYQGTPTELEKYVAPFRALGPVGETRTTAISYPDLFPLNRANETNLETCGKGLYRHLFPVGLKRYNPAAQRKIYNLFNNLTATYPSIAFTSTFVMEGYSTQAVTSVPDESTAFPFREDNILQSPVWGYTDPSLSAEIIAVSKEMRRILVDASGSNKLHAYVNYAFGDETLPEMYGHEPWRVKKLKQLKKKYDPNGRFNFYAPII

SEQ ID NO: 66 - protein, 500 aa, synthetic construct
LHPDPGNIRSPAAVDNDGLPISLQSCLGATGAEVVYPTDAEYANLTLPDNSNYHPHPQAIVIPKSTQEVAAIVRCVSAEQGRVTLSPRGGGHGYAAYGFSGQVVIDSSQMTDIVLNEPAQEVTVQMGQTIGILALTIGRAGYALPHGTCPGVGIAGHSLGGGWGYTSRQWGWLVDHLVSLELVDVAGRIHTLGPTTNTSDSDPDLWWALRGAGSNNFGIVTSFTYRMERAPTAITNYNIDFATHSDCVQALLTLQDLGSQPDGLPTSLGGELVITGGYTPPNAYCSFTGQYFGDPSSYNTTIQRFLAPLAQQSIQPQSTTSASFTNWVDALTDLMGPLDDPSIPQPYYAQSLLDPGTPNYTPTTASSILTAVQPAGPAAHISFDLNGPSSATNQPPQNDVGPMSFFHRKSLFLAQIYAADFPGFDDPGRKEALDTIWAISDAVRSADPEGKWHAYQNYIDPDLDNWGEMYYGANLERLKGVKERVDPNHVFDFPQGLGRA

SEQ ID NO: 67 - protein, 535 aa, synthetic construct
TRTPLNSCSPTDKRSNTSTETHASFRDCLLSELGRLNSFSSKTVLPGDISYPLSTLTYNLAIQHNSSAIVYVSTAKDVSKAIQCASNFRVPVVSRSGGHSYASFSSSPNGLIIDVTNLKDFSFETNGVGKVGELVTFGAGLRLGDLDMALQPHERAIPHGVCPYVGVAGHATCGGFGTASRMWGLFSDHVVEMEVVIANGTILKASEKINPDLFFVRIPPSYVHAMRGAGPSFGIITSLTLRTRQAPRNVILFEINYRFPNPDVAAATLNHFQSWGLKSAPSQLNIRWSVKLNHGPNSPKGVDLDWKLQGTFLGHMARFNRTINKLTKDFRVKGELVKLERLDWVESIWALGGDRTLSSEGINPSTYNASFYAKSFIVRDVSPLNYYEWKTFTEKLYGIASGIQKSKRLDWFLEIDLIGGRYQGQETGVRGLHSTTTSFGPRDALLLFQMGGNGPKGAMLYHGGLMESIRVNSEEIYSSLGGGQKELTGFNCYVDSEFSADRAHREYFGQKNTAILKNLKKVWDPSRVFNHPHSF

SEQ ID NO: 68 - protein, 521 aa, synthetic construct
QTGKTSPNVFEPDNFDINAALYNLGVDVSAIPALSSLQSRSTESACSAACSALGFLYGPSRAFAQNTTAYANATGSYWSVQQEEVRPYCIFKPSVNTDVSILVLLSRYTGCPFAIRSGGHAAFAGASSIQGGITVSLKDMSAITLNEDRSVVSVGPGNVWVQVYAALEPYGLAAIGGRVSSIGVGGLTTGGGISYYSNLYGWACDNVESFEVITASGAIVTASAHSYPDLYWALRGGGNNFGIVTKFNLYTIPSPEMRGGTRAFAEDQFSKVISAFISVVNDAPNDGNAQHWVAFIHTQGQNLAAVELTYVKNVTEPAIFAPYRAIPAIEDTTASKTLVEYCDTVQEVNPDGLREMYWTLTVRLDEAFANWVTGYFYSVLPQVWSIPGINPGLVNQGITIPMLKNMTRNGGNALGLDASEGPLHLLLMSIWWENADDDETIVAWAKNFWDTVSAKAKSDGVFHEYVYMNYASQYQDVVAGYGAANKAKLQSIAAKYDPKRVYQRLQPGYFKLNGAPASDAS

SEQ ID NO: 69 - protein, 513 aa, synthetic construct
APGSNFKALQQCVNGALAGGGNVEKRVQSPANATWDDAYIGAIIKPETPAMIAFAESFTEVGALMGCANTTGYQAVPRSGRHHFEGWSALNGSLVIDVSSLDYIKVSSDLSNATIGAGANLGQIYTELSVVNKTFLGGICPTVALGGYLGVGGYSLQHRALGIAVDQVLSFKAILASGELVTVSPTSYPDLWFAARGGGQYAFIVEATVKILTLPRSAMVVGFYNNSDTRYEVARKWLDWAPTAPKELTTQLNVYSNRTHLIGWYLGGSPEQLRTALNSSGLLDVADGVVSIDGNCSTANSRMYWQDPSTRCTDDASAYKAFLKVYNTESINLTPIQPAFRLDNTPALPSEPVAEPWPRFGVISKTYFTQKSRPITNDTLKELINSSASLGEEAGFWGEWTSFGIANPTTTSAFPWLKEAQALLRIEMNSPKNVTQYNQNRAWLLNFEKFFRPRVGNASYAGYVDADISINPLTAFYGNNTCRLISIKQKYDPNNFFRNPFSVPTQVPKGISC

SEQ ID NO: 70 - protein, 519 aa, synthetic construct
QACNNHQLTDAILQEFSQTLSGDLVLPTDGLYQWARLIHHTNFDGIYPVGIVFCETPEDVSKAILFARQFGLHVTARGGGHSYEGYSVTGGLLIDVSRMNSVSVNPAAMTAVVGAGAVLIDVYHGLYYPHKLSIPGGSCPSVGIAGYLLGGGVGLQSRTYGVGCDRVLEIGVVLASGEYVVASPTNHSDLYWAYRGGGGGNFGVVTHFKMQCHPVDRLSYAIITWDWEAAAPAFNAWQNWLKGLGEDYRNFSIFKFLVNAGGDGGLGPKVNLIVQFDSQSNTGTGELETLMAPLLNTAHEHIVSQTIMNGDFFEVTMEIMAGCAWPKTDLDTEFLRCHTVGNPAFPMASLPRDTYKAKSTFFADVISEKGIETCIKAIEDRFNSNLPDNSSQFTCALQFDSEGGIMGDVPKDATAFMHRDCLMHCQYLAYWPAEGDAWFDDNPDYSYCDVSNGSMQWISDAFNVLWPFGNGHAYQNYIDKEQPNWLYAYYGENVERLRTVKAKYDPDNIWKFEQSIPPA

SEQ ID NO: 71 - protein, 460 aa, synthetic construct
CSGTAPPAAAPQAPPSGPPGPSVPSGPPDWAALRSRLSGELLLPDQEGFTYQGFNPVWNQRTPAAVARCASVDDVRACVEVARSRVPIAARGGGHSYAGYSTPENGLQVDLRRLATVEVLPGDQVRVGAGAQLGEVAQVLAASGRCLPTGTCPTVGVAGLTLGGGIGVLARKYGLTCDHLVSATAVTADGRVVTASAEQEPDLFWALRGGGGGNFAIVTELVFATVPAPQVTVFSLQYNAGVADVLGAWQDWIRQAPNELWSNVVVRGGPEPTCRVGGCFVGPVGGLTSLLSALPAPASRFVQPQSYPEATQYFAGTGIQHESFVASSRIVRSPLTDPARAVALMNGQSAESYLIFDGLGGAVSDVAANATAFPHRGALACAQIYFKTPPAGATETAVAMGEVRDRLGELVGDSSYVNYIDPQLPRWAEAYYGDNLARLRDVARKYDPDRVFAFAQSIPR

SEQ ID NO: 72 - protein, 468 aa, synthetic construct
CSPVLPNEIPAGVRWEDLRRSLSGALVVRGDAAMDESGRAFNPLFDVNHPGAVAFCASDQDVARCVEFATSARLPIAARGGGHSYAAYCIPNDGLVVDLARMAAVSVTGTRAVVGAGARLIDVYAGIAGAGRMLAGGSCPTVGIAGLTLGGGVGVLTRRFGLTCDQLISARVVTADGKIRVVSADTESDLFWAIRGVGGGNFCIATELAFETAASTDLTVFTLDYAAGEMAPIVHRWLTFMAGAPDELWTTLHAIGGAIPQCRIVGCVAQGVNSQDVIESLRGGIGVRAADSFIAEMTFLDAMKFMGGCTTLTVAQCHPSWTGPGSGQLKREAFVASSRMVPHPDVDTARIETLLAGKPGLTFIFDSLGGAVRRISPDATAFPHRQAAACIQIYHGVGADPAVAHERVSQARDGLGDICGPAAYVNYIDPGMPDWATAYYGDNLPRLRGIAAAYDPKGIFRFAQAVRP

SEQ ID NO: 73 - protein, 494 aa, synthetic construct
GGSAGSKIASSSVVEVPPAPTVVSKLSAEQWQGLSEQLNGELILPTNVVDYSQERLVFHTRFDHIYPQAVAKCRTTDDVIAVLSFVKQHDLHIVPRSGRHGYAGYSTTEGVVLDVSQLNSISIDDGYATIGAGARLVNIYDQLTNQSVCIPAGTCLSVGIAGLTLGGGFSVIGRAHGLTCDVLESAQIVTAQGELLTCNNNENSDLFWALRGGGGGNFGIVTSFVFRTHKTQDINVFEASFSFNDFEQLMSKWQVLAQSWPDEMWAQCLPNWINGSVRVTVRAFCLNQADEARSYWEQFIAAVHTSPLSNNESTESYRDIMLGNCVNNIPSCHLSVQSAGGRMQRSAFGASSDFFEGIIPPSGIKTLKGFIEKSINEGKYGMIIFNLLGGAIKHTEVTETAYPHRRANISAEYYAPINISSTNEQIDETQKWQNQFREIMAPWSTGGAYVNYMDPLILDWQQAYYAGNYYQLQKIKSKYDPEEMFNFTQSVELA

SEQ ID NO: 74 - protein, 584 aa, synthetic construct
CATSRPAPQPEAAPLCRCAPGQPCWPTRDDWQKFGASLRGKLEQPQSPFAPCRSDAAGEACATAIRNSTNPFYLQDQPGGTESTGWLGAWTAASSAYAVVAKDASDVVAAVKFARRSRLRLVIKGTGHDYLGRSNAPDSLLVWTHQMRKVDVRDAFIPQGCGAAEPAPAVTLEAGTRWLEAYQEVTVKHGRYVQGGGCTTVGAAGGFMQGGGFGSWSKKYGIAAASMLEAEVVTADGRVLVANGCQNQDLFWALRGGGGGTFGVVTRVTLRTHPLPDYFGAIDGSIVAKTDAAFKDLLEHFLLFYREELSNEHWGEQVKVRRSNSLDLSLVFEGMSAKQAGEVWRPFREWVEANRDTLTMKAELVEMPGNRMWDAAYLKNIPGAIETDPRPDQPPDRFWWWAGDGGQVSAYWYAYQSRWVPLDLFEPANASGFAATLFDASRHWSVALHFNKGQAGASAEARERGREISMNPAVFKAAALIIIAAAANAYPGVPGHEPDMTEGESAKARVGAAMKVIRAATPGAGSYLNETDYFESDWQREFWGENYARLSEIKRKYDPDGLFFCHHCVGSEGWSEDGSCRSTR

SEQ ID NO: 75 - protein, 573 aa, synthetic construct
VSDKNLKPNCRCLPSEPCWPDQQQWDTLAKSLKGKLIKPRLPLSVCEKNSDSKECKLVLQNIKNPFYMQSDSGRNESQGWYGAWHNQSSSYAVEAEDTQDIVKAANFARKHNLRLVIKGAGHDYLGRSSSPDALLIWTHNMRDIEYNKHFHPQNCPKNKEYSAVTVGAGTRWLEAYNVVTNHHHQYVQGGGCTTVGAAGGFPQGGGFGSWSKQYGTGAGGIIQAEVVTADGKVVIANECQNQVLFWAIKGGGGGTYGVVTNLTLRTHKLPSHFGLISGEITTDSDKAYKKLIKEFLLFYSSKLNNEHWGEQFAFRPNDKMTIAMVTQGLAEKEALDIWQPFKKWLESQPDLHYKLKYIDIPPTQIWNYDFWHKNYPDMVIKNIGPDARDGEFWWASNTAEVSAYWYTYQSWYLPEKLFDSKNIDKTVDTFYKVSQLAPVSIQINKGLAGASKEAIELTKQTSMHPGVYDAGALAIMSYSNDKPEFDKPKMTQEIEQKVDNIYKAMNMIMALAPDAGTYANEADYFQKNWQQVFWGSNYSKLLKIKNKYDPNGLFYCHHCVGSEYWQQDGMCRK

SEQ ID NO: 76 - protein, 474 aa, synthetic construct
ATSQWNILRSRLQGTLVLPTDSGFAAAKQLQLGQYDAVNPQAVAYCQNADDVRTCVLFSQYTGTPLRVRSGGHNLAGWSTGDNALIVDLSRINQATVTGSTVQLGPGTQSIDALSQLKPYNKQVIAGTCPTVCAGGFLSGGGLGWQTRKFGIGADHVVSARVATSDGRITYCSPFVEPDLYWAVRGGGGGNFGIVVNFEVTPIDAPVLTAYETVWSFDNAATTIAAWEHWTANAPRELGSSLIVVPPNGYYGYPSSNPLVRIYGGYHGVQADADAQLDQLALAVGAKPLSRTSQQFGFADGMQYLYGCGDITELACHRAGSNPDAVLPRTPNQRQSYRLIDRQLTTADATDVLNAWSAHPELPYRFLQSFALGGAANDVPSGATAYVHRSAQSLLGYQVGFQDSTDVPAADDWAAAGAAALAPRAGGSYINFPSSVMNGDWAADNYGDNYARLVQVKYAYDPTDLFQHPQSVGS

SEQ ID NO: 77 - protein, 598 aa, synthetic construct
RTTFRRRRPSDASWPSPSAWKQLNDAVDGNLSPVSSPLSVLKADPAGAAKRVWQNLKNPYYIGDEPGLTQTLGLVDAWTTAPSVYAVAARNAHDIAAAVNFARDNDLRLVVKGGGHSYQGTSNAPDSLLVWTRHMREAAMHAAFVPQGCEGTVPAQPAVTFGAGTMWGHAYDAVTTKGGKYVQGGGCLTVGVPGLIQSGGFGSFSKHYGLAASSLLEAEVVTADGRIRIANARTEPDLFWALKGGGGGSYGVVSKVTLRLHDLPEFFGAATFTVKADSDEAYRRLIREFVGFYHEHLFDDHWGETVHVKPDNSLVVGMSSLGLDTGQARRIWQPFLDWVANSGRACSVEGRAVIASLPARRFWDLQWWREHWPELGFPPNGRRVIAWFDAVLAYVLRQPLFEIDPRPGAAPNNAWWTANAGEVAAFLWAYESLWLPASLLEKDAQQPLADALFAASRYSAIELHFNKGLAGAPSAALSGAADTATNPAVLSAFALAIAARGQGPAYPGIAGHEPSVAEARKAAERIGRCMSQLRALVPTPAAYVSESNYFEQGWQRSYWGSNHPRLAAIKRRYDPDGLFFVHNGVGSEQWSADGFTKL

SEQ ID NO: 78 - protein, 468 aa, synthetic construct
FGFELPAITRTGAETTITHAEVKELAKSLTLALLTPDSHRYHENRKIWNGMWDDKHPALIVQCMDVSDVINAVNFARTHDLLLAVRGGGHSISGKSVCHGGMVIDLSYMRSVQIDPYRKTARVGGGALLSDLDRAAQRFGLVTPSGVVSHTGVAGLTLGGGIGRLMRKYGLTIDNLLSVEIVTPDGRLQRASDQENADLFWAVRGGGGNFGVVTAFEFRLHSMGTEILSCGLAYPLDQAKDVFKFYFDFSREMPDELHFGLSAAIQENGDSVGLFFGLGYSGSLKEAERIIEPIRKFGKPSFEAIGMRNFVELQSMGDAHNEHGRTYYIKGRHMNDFDPDMVDPFIEQFQHAPSRFTVMRIVRFGGAIERVAKDATAWPHRDAKWDFEVGGSWPNPDLSEKYVQWGRDYWESLDSYMADSFYVNELMDEGQSHVNANYQGNYKRLSKLKQKYDPKNLFRLNANIEPSG

SEQ ID NO: 79 - protein, 504 aa, synthetic construct
LPLAADPTGPKDCAGILPPLSALAGVPAAVAEPNWAQRAGTINDASCLNRTPVAGVVAVRNEADVARALAYARAHKLTVTAAGMKHSMGGQAFRAGGVILDMRGLNAIRLDANTRTVSVGSGATWHAIQNAIHPQFAVKAMQSTDIFTVGGSISVNAHGMDHQSGAVMGSIRSLRLMLADGRIVTASRTENADLFAHVVGGYGLFGVLLSAELEVVPNDVYRSERSLISYRDFPETFAAIEADPAIGLTYTHLSTAPGSLLQEALVYAYRKVPADQSLPRAPLGEVSSTKLRRLTVNLSKRNDFFKAAKWWAEKNVEHRLESCTIPRAQAMGDGEACLVSRNDPMHDSVAYLRDALPRETDILHEYFVPRDRLVPFIDGMRALFRQQDVNLVNASIRAVGAEVNALSYAPKPAFSVVLYLNQPADAGGTAAMARLTSDLIDLCDREGGRFFLPYQLHYTTAQLVRSYPELPAFLAEKRRWDPDGLFGNTWYARYAPAVTGLQTG

SEQ ID NO: 80 - protein, 456 aa, synthetic construct
DHRIAPWDELASKLQGALVRPGAASFSELSTPWALQYASTIPQAIAQCRTEADVQTCIQWAKANSMPIVARSGGHSYGGYSLTTGLMINVSALNTFTYEPTTGLATVGSGARNRTIFTQCKPLDRAIPHGRCYEVGVAGLVLGGGIGFDMRLHGLTCDALRETRIVLADGRMLTCNEKENADLFWACRGAGGGNFGIHTSFTFDTFPVGTITTFNLVWTDRLADTFAAIQTMTLSAPDTLGIKVSITAQKQAGASVLSLSILGHFVGSEADLRSLIAPILTVQKPAEQTIQIAPYWDGQQLITEEGRPEYSHERSRFVKGQLSDEAVRQIILNLKNWPGTSKAATWKYFLLGGQIDARSPTDMAFVHRGYSMLSSIELEWTPADDAALLAKNEQWLTGFHNQMEAYTSSYCYQNFIDPSQEGYLQAYYGTNLPRLQAIKRKYDPENLFNYPQSIPV

SEQ ID NO: 81 - protein, 532 aa, synthetic construct
MKWKSKKRKDAELRVAGWDALPSVDLLFSDKDAHTLRALRAQKPIPLDQRFSVDDVKDLQKAIHGTVVLPTDLTYHRDRQMTLLAVQEFPQLIVFCEIFEDARRCLEFAQKYDLRVTIRSGGHSTAGFSVNSGMLIDTSRIKHVVVDPKEKRAIVGAGATIGDLNAALKMYNLHVPSGGCGDVCIAGYMQGGGYGFTARKFGMNCDNVHEIVVMLADRTIVVANSEKNADLFWAVRGGTGGNFGILLQVTYNLHELGDVWGFGLRWSIDSRDGIKNAAAALAEMQANYMQTGAPPELGFMTFLAWQDETPCVVMRGMYLGPQRDGEFAIDSLRRLGPVYTDFGVGRYADLDLKLIESPPGLPQVPDLAREEKQCGYIDKKLDAEDWAHIIGVFLTSPNRSSLVCIEPYGGRINEIPATSNAFIHRKVDMNLFLDVFWMSEDQQRKDAIAFLDRFMAMMAPYFNDHSYQNYPRRTQQDFRRRYWGSSFERLLAVKLKYDKNSFFQYAQSVSRETPETHYDLPQLPEGIETEPY

SEQ ID NO: 82 - protein, 489 aa, synthetic construct
MEHPLRVDEFLPSDAPVPAADAEIAAPPAEGVAPLPPLPNLPVTDVEFLRPQDAHYADYLPAASKRKQLAPALRAVCKTEHAVAVMVDWVRSNGLSFAVRCGGHSYEGLSQSSGVAIDVRGLKQIVVDKASNLVTAGSGVSLYELYSALAAQGLALQAGSCPTVGISGHLTGGGHGLLARSHGLTCDGLLQANVVDTQARVLQANATSEPDLWWACRGGGGGSFGIATEFRIEVFPLKTTRVFGVSWKLSQAHAAQLFAAWQDWAPNAPSNITSIMKVGPAGHGLITMRCIGQSVGGESELRSELRRLTAVRPPSSALSVQSLAFLDAVKHFAGPLAYESVLMKAKSDYVLTPLASDGIEAMMAAVAPIVSGGIVLLCDSYGGRIADVAADATAFPRRAGTQYCIQYFSSWSRSADTAAHLADVARVYAAMRPFMPGASYVNYCDLDLDDYASAYWGDNLARLVAVKQQYDPDDLFHHAQSVPLSVPTA

SEQ ID NO: 83 - protein, 500 aa, synthetic construct
MSGIRRRPETDESCRTAPAATEDLPMTATPASSPPENTERLTVRTRTSVLAQLRDRIAGDVYGPGDAGYVDARSGFSRLDVHRPAVIAVPANHFDVVEAVRFAVEEDLRVAVQATGHGTGRPADGGLLINTSRLASVSVDPVTRTARVGAGATWRTVVDAATPYGLAPLMGSASGVGAIGYTLGGGFGWLGRRFGLASDHVLSFDLVTPDGTPIRVTDTSFPHIFWALKGAGAGGLGVVTSMEISLVEVSTVYAGNLFYPAADAEEIIRRFRDWAPAQSDALTSAVAILNLPPTDDVPEPLRGRSFAVVRGCWSGDLDAGRAVVDEWRDWRTPVVDMWDVLPFAAIDAVSMDPTEPMPVMVTTEWFDDLPDVAVDIVAARVRPAPASAPLIVIGEIRHAGGAIARGAEHAPNDRARTGRFLLELVAAVPDPQVALAVESTLRVTRTELAPYVTGAAYLNFTSGTERVARADDAYSSDHRRRLDEIKAALDPSNRFCHGDI

SEQ ID NO: 84 - protein, 508 aa, synthetic construct
MLALQNGNQFKKHPMEKITANDPRYPDLAGKRFNKRFSGKPEYICLPTTTVQVVEAVQEAVDNNKRLVVRSGGHCLEGFVTDPAIQVVLDLSLMTNIAYDTDKSAFMVEAGATVGEMYRRLFLGWGVVVPAGEHPGIGVGGHVVGGAFGFICREYGLAADYLYGVEVVTVNTSGKAQRAIATRNTNDPNRELWWAHTGGGGGNFGIVTRYWFRTPLNTGSDPFTALPPAPAAVTTFRVAWDWKNIDKASFTHLIRYFGEWCQQNSGPDSPYRQLFSLLFLNHRNIGTLEMKGGATGPAAKQLIEEHLAAIADPLGLSYTPQIEELPWLRFALNPFPELFQPGFDDAKAKIKDALLRRPLDDSQIATAYKYLTMEGNMGVLGMATYGGKVNAIAPAITASAQRDCILDIACNTGWGDPAEEASAVAWVRHFYRELFAHTGGVPVPNEQAGGAMINHPDTDLADPEWNKSGVPWHALYYKENYPRLQKVKNQWDPLNIFHHALSIKAVDQ

SEQ ID NO: 85 - protein, 195 aa, synthetic construct
MGSLRRALVSADRLLQSSAAIHRRVSIAAANSLVRAPESLCRPRSSVAYSGVYRFYSNNYTNLQSLDIDLSNEENKKRLYNRLVYRSKQRGFLELDLVLGKWVEEHISSMDENGIIALVHVLDLENPDLWKWITGQEQPPEALQINPVFNALRDKVMNNLNTHSSPETRAKPGQPWVRGWDDIKRARDSPITGNQ

SEQ ID NO: 86 - protein, 241 aa, synthetic construct
MEKMIPLRSLYRSLWTRSYHMAALNHNGLRHYYAARCLPNFSSPSTSVTSRQALVSSDCRSPLSLSMGSTRSYSEDVTHMPVVTDPDLNRAFKDLLAASWDDLPDSLVHEVKAALSKNTDDNAGKEALTNVFRAAVASEEFGEIIMNLKMEFDDTVGMSGENIKPLSKEHANALQTIFSRYNTYLEAFGPDETYLRKKVETELGSKMIYLKMRCGGLSSDWGKVSVLGTSGLAGSYVEKRA

SEQ ID NO: 87 - protein, 191 aa, synthetic construct
MGSLRRALVSIDRLLQSSAAIHRTISIAAANSLVRAPESFCRPRSSVAYSGGYRFYSNNYTNLQSLDIDLSNEENKKRLYNRSKQRGFLELDLVLGKWVEEHISSMDENGIKALVHVLDLENPDLWKWITGQEQPPEALQINPVFNALRDKVMNNLNTHSSPETRAKPGQPWVRGWDDIKRARDSPITGNQ

SEQ ID NO: 88 - protein, 510 aa, synthetic construct
MEIDKAIRESDDRWLKTKYNKAIYVIQRALALYSIEEVAFSFNGGKDSTVLLHLLRAGYFLHKGGQNCANGGPSDFPIRTIYFESSSAFPEINSFTYDAAANYGLQMDIIRTDFKSGLEALLKANPIRAIFLGVRIGDPTAVGQEQFSPSSVGWPPFMRVNPILDWSYRDVWAFLLACKVQYCSLYDQGYTSIGSIHDTVPNALLSVSNSLSSEEKFRPAYLLSDGRLERAGRVNKFSSGSGHSPSFSNGLISADLHKNSLLTASAIAVGDEILLGIVEDQLGPLLCKKLHSIGWSMSQTVVVRNDVDSVAEEVERKSSTNDLVFIYGGIGPLHSDLTLAGVAKAFGVRLAPDEEFEEYLRSVIGDYCPSDRNEMALLPEGITELLHHEKLAVPLIKCKNIIVLTATNFMELETQWDCLIELTRSSDILAVMEPYVSRLLKTDLSDVATAKPLSKLCLEFPDLHISCYRQTRYGPLIVNIRGRDEARIEAAVEALSKKLHPGAFSEINSS

SEQ ID NO: 89 - protein, 367 aa, synthetic construct
MDGNKEDALKCLRIGKEAMEAGDRNRARKFFTKAQRLDPTLSVDDLLSAVEKESGGERSPSDADASSNGPSDSTSSKPSDQPSLRQRVPSTGSSSSPSSATGAHTLEQIAIVREIKKKKDYYEILGVEKSCSVEDVRKAYRKLSLKVHPDKNKAPGAEEAFKSVSKAFQCLSSEESRRKYDLVGSDEPVYERRTTRHAAHGFNGFYDADVDAEEIFRNFFFGGMAPATQFRGFSFGTGMGHRQQQPDHNGSGGFNVRALIQILPVLLILVLNFLPSSEPIYSLSRSYPYEYKFTTQKGVNYYVKSTKFEQDYPLDSRERSSFEDQVEREYFGILNQNCRLEIQRRQWGFIRETPHCDMLQRFQTQAN

SEQ ID NO: 90 - protein, 700 aa, synthetic construct
MNPDKFTHKTNEAIAAAHELAMSGSHAQFTPLHLAVSLINDPSGIFSQAVANASGSEDAPKSVERVFSQALKKLPSQSPAPDDIPASTSLIKVIRRAQAAQKSRGDTHLAVDQLILGLLEDSQIGDLLKEAGVATARVKSEVEKLRGKDGKKVESASGDTNFQALKTYGRDLVEQAGKLDPVIGRDEEIRRVVRILSRRTKNNPVLIGEPGVGKTAVVEGLAQRIVRGDVPSNLADVRLIALDMGALVAGAKYRGEFEERLKSVLKEVEEAEGKVILFIDEIHLVLGAGRTEGSMDAANLFKPMLARGQLRCIGATTLEEYRKYVEKDAAFERRFQQVYVAEPSVPDTISILRGLKERYEGHHGVRIQDRALVIAAQLSSRYITGRHLPDKAIDLVDEACANVRVQLDSQPEEIDNLERKRMQLEVELHALEKEKDKASKARLVEVRKELDDLRDKLQPLMMKYRKEKERIDEIRRLKQKREELLIALQEAERRYDLARAADLRYGAIQEVESAIAQLEGTTDENLMLTETVGPDQIAEVVSRWTGIPVTRLGQDDKDRLIGLADRLHKRVVGQDQAVAAVAEAVLRSRAGLGRPQQPTGSFLFLGPTGVGKTELAKALAEQLFDDENLLVRIDMSEYMEQHSVSRLIGAPPGYIGHEEGGQLTEAVRRRPYSVVLFDEVEKAHVSVFNTLLQVLDDGRL

SEQ ID NO: 91 - protein, 704 aa, synthetic construct
MMNVQMGDVETFAFQAEINQLLSLIINAFYSNKEIILRELISNSSDALDKIRFEGLTDKSKLDAQPELFIRLVPDKANKTLSIIDSGIGMTKADLVNNLGTIARSGTIEFMEALQAGADVSMIGQFGVGFYSAYLVAEKVIVTTKHNDDEQYIWESQAGGSFTVTRDVDGEQLGRGTKITLFLKEDQLEYLEERRIKDLVKKHSEFISYPIYLWTEKTTEKEISDDEDEGTKKEEEGDIEEVDEEKEKDSKKKKKVKEVSHEWQLINKQKPIWLRKPEEITKDEYASFYKSITNDWEDHLAVKHFSVEGQLEFKAILFVPKRAPFDLFDTRKKMNNIKLYVRRVFIMDNCEELIPEYLGFIKGVVDSDDLPLNISREMLQQNKILKVIRKNLVKKCIEMFNEIAENKEDYAKFYDAFSKNLKLGIHEDSQNRAKLADLLRYHSTKSGDEMTSLKDYVTRMKEGQKDIFYITGESKKAVENSPFLERLKKRGYEVLYMVDAIDEYAVGQLKEYDGKKLVSATKEGLKLDDETEEEKKKREEKKKSFENLCKTIKDILGDKVEKVVVSDRIVDSPCCLVTGEYGWTANMERIMKAQALRDSSMSSYMSSKKTMEINPDNGIMEELRKRAEADKNDKSVKDLVLLLFETALLTSGFSLDDPNTFAARIHRMLKLGLSIDEEEVEGDEVEMPPLEEDGNEESKMEEVD

SEQ ID NO: 92 - protein, 15 aa, synthetic construct
MKSLLLSLLAVPATA

SEQ ID NO: 93 - protein, 18 aa, synthetic construct
MKFSAVSIAAALASLVAA

SEQ ID NO: 94 - protein, 19 aa, synthetic construct
MKLSTILFTACATLALALA

SEQ ID NO: 95 - protein, 17 aa, synthetic construct
MKVLALLVTVCFSVASA

SEQ ID NO: 96 - protein, 24 aa, synthetic construct
MIFDGTTMSIAIGLLSTLGIGAEA

SEQ ID NO: 97 - protein, 30 aa, synthetic construct
MKFSMPSWGVVFYALLVCLLPFLSKAGVQA

SEQ ID NO: 98 - protein, 874 aa, synthetic construct
MKLSTILFTACATLAAALPSPITPSEAAVLQKRVYTSTETSHIDQESYNFFEKYARLANIGYCVGPGTKIFKPFNCGLQCAHFPNVELIEEFHDPRLIFDVSGYLAVDHASKQIYLVIRGTHSLEDVITDIRIMQAPLTNFDLAANISSTATCDDCLVHNGFIQSYNNTYNQIGPKLDSVIEQYPDYQIAVTGHSLGGAAALLFGINLKVNGHDPLVVTLGQPIVGNAGFANWVDKLFFGQENPDVSKVSKDRKLYRITHRGDIVPQVPFWDGYQHCSGEVFIDWPLIHPPLSNVVMCQGQSNKQCSAGNTLLQQVNVIGNHLQYFVTEGVCGIGHHHHHHMDSPDLENLYFQSNPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHHHHH

SEQ ID NO: 99 - protein, 744 aa, synthetic construct
MNPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHHDDDDKMKFTAATLLLAAAASALDVVTDGSKPFGIMSLRSASAIHLSSVGVSGDALTIGSSGAKFTIKDGVLYADDKAIDFSSGEAKVASDGKGTSGVTLEKGYVTVPGFNWAGCPEGNGYAVDDNSKCEDDGIPFGAYAVADTSAESSAAPASSAAAAESSAAPSSAAEAKPTAGGNTGAVVTQIGDGQIQAPPSAPPAAPEQANGAVSVGVSAAALGVAAAALLI

SEQ ID NO: 100 - protein, 334 aa, synthetic construct
MKLSTILFTACATLAAALPSPITPSEAAVLQKRVYTSTETSHIDQESYNFFEKYARLANIGYCVGPGTKIFKPFNCGLQCAHFPNVELIEEFHDPRLIFDVSGYLAVDHASKQIYLVIRGTHSLEDVITDIRIMQAPLTNFDLAANISSTATCDDCLVHNGFIQSYNNTYNQIGPKLDSVIEQYPDYQIAVTGHSLGGAAALLFGINLKVNGHDPLVVTLGQPIVGNAGFANWVDKLFFGQENPDVSKVSKDRKLYRITHRGDIVPQVPFWDGYQHCSGEVFIDWPLIHPPLSNVVMCQGQSNKQCSAGNTLLQQVNVIGNHLQYFVTEGVCGI

SEQ ID NO: 101 - protein, 20 aa, synthetic construct
GHHHHHHMDSPDLENLYFQS

SEQ ID NO: 102 - protein, 5 aa, synthetic construct
DDDDK

SEQ ID NO: 103 - protein, 221 aa, synthetic construct
MKFTAATLLLAAAASALDVVTDGSKPFGIMSLRSASAIHLSSVGVSGDALTIGSSGAKFTIKDGVLYADDKAIDFSSGEAKVASDGKGTSGVTLEKGYVTVPGFNWAGCPEGNGYAVDDNSKCEDDGIPFGAYAVADTSAESSAAPASSAAAAESSAAPSSAAEAKPTAGGNTGAVVTQIGDGQIQAPPSAPPAAPEQANGAVSVGVSAAALGVAAAALLI

SEQ ID NO: 104 - protein, 260 aa, synthetic construct
MIVGPKTKFHSLYNHMFSQTCHKFSGVVDSFLASNGGALRKATQDHVRESLRVLHEALERYDLNQISLSYNGGKDCQVMVILLLAALWRRFGEDPAVLNNLGAFKSVYVASEKAFEEVDTFVDNSCQEYGLQQIRLSEPMKAAFEHFLSENPTVKAIIVGIRRSDPYGQQLKPFDPTDSGWPDFMRVHPVLEWKYVNIWDFLRGTDSAYCCLYDKGYTSLGGTDSTVPNPKLLKKGTCAEFLPAYALVKDEEERLGRFRK

SEQ ID NO: 105 - protein, 305 aa, synthetic construct
MSIKREESFTPTPEDLGSPLTADSPGSPESGDKRKKDLTLPLPAGALPPRKRAKTENEKEQRRIERIMRNRQAAHASREKKRRHLEDLEKKCSELSSENNDLHHQVTESKKTNMHLMEQHYSLVAKLQQLSSLVNMAKSSGALAGVDVPDMSDVSMAPKLEMPTAAPSQPMGLASAPTLFNHDNETVVPDSPIVKTEEVDSTNFLLHTESSSPPELAESTGSGSPSSTLSCDETDYLVDRARHPAASAQDFIFIKDEPVDDELGLHGLSDDFTLFEDNKQPAQHDFIADLAHYESSVSNLFGGLE

SEQ ID NO: 106 - protein, 495 aa, synthetic construct
MNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGTCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 107 - protein, 477 aa, synthetic construct
MATPSAFSGSVLTPGDDGFEAAQVTWNACYSSRPREVMVCHDAASVAEAVRSVRERGLPFRVRSGGHSMCGLSNLDDGVIIDLGGLGGVELTPDRQTVRIGGGARLADVYNTLWDHRLTVPAGTCPRIGVGGHVLGGGMGVLSRSRGALVDHLTALEMVDAEGRLLRVSEDENPDLFWACRGGGGGNFGIVTAYELRPTPIDDVTIFTVSWTWSQLPDAVRAWQRWLGSAESRINSFLSLFPQQQDMVVAFGVFDGPAADFRPLLAPLTAEVAPEAEVVEEVPFIQAVDTVEALQGEAAAAEQVRAQGSSAIIANPLNDEALATLQEFLTDPPSHRAEVAVYGMGGVIGERERGDTAFVHRTGLMAFNYRTDWDSPEDDRLNLDWVTRLRHAMAEHTTGAAYVNTIDLALENWLWAYYEENLPRLMAVKRRYDPENVFHHPHSIPGSLTAEAARAHGVPEATLKRLHDDGLLDGPLD

SEQ ID NO: 108 - protein, 476 aa, synthetic construct
ATSARFAGTVLTPGDPGFEDASRTWNACFASRPREVMVCHSTAAVADALREVRERGVPFRVRSGGHSMGGLSSVEDGVVIDLGGLDSISLSEDRKLVTVGGGTRLGDIYRELWRFGVTVPAGTCPRIGIGGHVLGGGMGILSRSRGALVDHLAALEFVDAEGRTYTVDENEHPDLFWACRGGGGGSYGIATSYTLRTSPVDEVTLFTFSWSWDQFAPAVRAWQSWLASTDDRVNTFLSLFPREQDLVAAFGVYDGPAAEFEELMRPLVSAVPPQDQVVEPMPFIQAVDTVEALQGEAVAADQVRAQGSSAIVAEPADDAAIATLQTFLTDPPSHRAEVAVYGMGGAIGRRARTDMAFVHRTGLMAFEYRTDWDRPEDDGKNLAWVEALRHAMAPHTTGAAYVNTIDMALENWLWAYYEENLPRLMEVKRRYDPGDVFHHAQSIPLSLTEADIARYGIPAPVAARLRAAGLTSDQEH

SEQ ID NO: 109 - protein, 467 aa, synthetic construct
MVTRSAHVAATVTAADPGYDEARRVWNERFDCRPRAVLYPADVAEVTVAIRAARDEGVPFRIRSGGHNVEGFSVLDDGYVIDLSRLTGVRVSDDRARGTVVAGTLLGDVYSGLWAAGVTIPAGVCPDIRIGGHVLGGGVGMLVRSRGMLIDSLVGLTMVDADGEVLEADDDSHPDLMWASRGGGGGNFGVATSYTFHTRPIGDVTLVTATWDSMNAITALDRWQHWMVDADKRVNARFSLFSPATGGGLMAALFEGTRAEWESLARPLLAQCPPTDYGIRETAYINSVGTFSQLVPSIRAKFVPALAAGPLDAEALKILERRHRDAPDGVKTGFYGLGGAVLGAGLAEKSAFAHRSSLFCVEYLGHWKGAENDAEHLGWLAGIRDELRPFMTGGAYVNSPDRDLSDWLHAYYGASLPRLMDVKRRYDPEDVFRFEQSIPVSLSPSAARAGGLTAPVIADLSARGLLT

SEQ ID NO: 110 - protein, 455 aa, synthetic construct
MATGAAVVAGDAVADARPARWARLRAHLAGELLLPGDDGYDTARLPFNSLFDHHRPAGIARCVRPEDVQRCLEEARRSGIRVAARSGGHGYAAYSTPHDGLVVDLAPMAGVEVRPDGTAVVGAGARLGDVYAALAAAGRCLPLGNCPSVGIAGFTLGGGLGWLQRTYGLACDWLRAARVITVDSRVVTASEDREPDLFWALRGGGGGNFGVVTSFGFATVPVPEVLTVFRVRFPAGSAADVLGGWQPWSVALPEEVSSSCVAMPDGAVDVFGCAVGREGMVLGELAHLLDAVGVDAPIEHAEVDYPTATAIMTGGQPTRNAFRASSRIAERWDDPAAVADTVRGMDSALLIVGALGGAIDRRTPTDTAYPHRGAPTSVEVFGTVGDLPPEQVTREVDQAQSALAGLIGTGTYINYLDPEQQDWARTAYRHNLRRLRAVARRYDPDRVLRFPQAVR

SEQ ID NO: 111 - protein, 451 aa, synthetic construct
MEKTKLTGRIVTRDDPGYNDARTNINLSLQRYPRLIVFCQNKHDAVNALKWARENNVPFRIRGGRHSYENFSLLNNGLVIDLSEMNRIKVDHNKQLATIEAGAELGEVYRTLWRYGLTLPAGNIANVGLTGLTLGGGIGYLTRSAGLTCDHLVKLEMITAEGKEEAELITVSRSEHPDLFWASQGGGGGNFGIVTSMTFKAVPISRVSIFSITWGWKDFEEVFNTWQRWAPFTDERLTSSIEFWPEEVNRIEALGQFVGTKTELKELLKPLLKAGKPTSGIVKTVPFIEAAAFFNSPGGNRPQNLKRSGSFIEKPLSNHAISTIKHFLQQAPNQNASVWQQSLGGAAGHIAPDETAFFYRDAIIAQEYLTNWTSPEEKSQNVRWIEGLRTALSKETMGDYVNWPDLAIRNWQKTYYGENFERLREVKTKYDPDNVFRFEQSIPPLRRSIFF

SEQ ID NO: 112 - protein, 473 aa, synthetic construct
MPAARILRRITRSDREFEALCRGWNRRFHADPLEIVFPASTDEVAAALLETQERGIPFRIRSGGHSVDGFSGIADGIVIDLRDLNTVDISADRTHVRVGAGAHLHDVYEVLGQIDRVIPGGVGRSVGMGGLITGGGLGGLTRWLGALAHNVVSFDLVDAQGEIHRVTPDSHPDLYWACLGAGGGNFGIITAFTLRMTPLSAAVWYRLSWPWSQFEDVYGAWQQWAPTADSRLTPLLVMWPTTSANRVDVAGIFPGTPEQLEDLLEPLLESVPPPSERQITPTTFVAAMAALLEKIGGESFGVGTRAVTASPFFDRPLDRNLMRILRDWHTGAPGNSFVWCLPGGAELATAAARGMQSFAHGQMQQLMLIRSDWTDVREDDACIAWVSGLYELMQPYACGAYLNWTNTDIESRPHRLYRDSYARLVQIKHRYDPNSIFSFTHAIPASISRVQARQLQLPDSTLAELSHRGVLVD

SEQ ID NO: 113 - protein, 462 aa, synthetic construct
MRTRTLARLESLRDAVRGSVLLPGDAGYDQERTGFQSGDRHGPDVLVAARTPEDVRAAVDHARRRGMPLAVQATGHGLRAAVREGVLVGTRRMTGIRIDAAARTAWVEPGVRAGDLVAAAARHGLAPVNGSAPGVGVVGYTLGGGVGLLGREFGYAADLVRRLDVVTADARLRRVTAERCPDLFWGLRGGGGGLGAVTGLELGLVPVERFFGGRLVFDGGQVAEEVLRTWQEWTRSVPDTLTSAVTLLPMPDLPAVPAALRGRYLAQVHLAFTGPREEGERLVRPLREIGALLVDEVRERPYTECATVFSEPETPHAYRSSNVLLGAAGLDDGARRAVPAEAGPSAAAMCVLGVRHLGGAFSRPPEVPNAVDHRDARYLVYVLSPVPEPDRDGAVRSLHARVLAPVTGPAPARSGNFLHGPQEPATLRSVHGDETAARLERLRSAYDPQGLFRGIGRFAGPG

SEQ ID NO: 114 - protein, 479 aa, synthetic construct
MNDMSLTNLQAGKTTISAAAIEALAGRLRGRVLDARDPAYDEARTIWNAMVDRRPGLIVQAAGASDVINAVRFAAENQLLVAVRGGGHNIAGNAVCDGGLMIDLSPMKSVRVDQTTKRAWVEPGATLADVDKETQAFGLALPTGINSTTGIAGLTLGGGFGWTTRKLGLTIDNLLSADVVTANGELVRASQTEHRDLFWGLRGGGGNFGVVTAFEFQLHELGPEVLAGLVVHPFADAENVLRQYRQALENAPDELTCWAVMRQAPPLPFLPEEWHGREILALAMCYCGDLEAGQKATAGLRGIGKPIADVVGPAPFAAWQQAFDPLLAPGARNYWKSHDFMELSDQTIGILTDAVRQLPGPECEIFVGHVGGAAGRVAAEETAFPQRSSHFVMNVHARWREPQMDQACIEWARRLFEAAKPHAVGTAYINFMPEDEGDRVEAAYAGNYRRLLEVKGRYDPQNLFRMNQNVRPAGLRGAA

SEQ ID NO: 115 - protein, 475 aa, synthetic construct
MATSARFAGTVLAPGDPGFEDASRTWNACFASRPREVMVCHSTAEVADAVREVRERGVSFRVRSGGHSMAGLSSVEDGVVIDLGGLDAISLSADRSLVTVGGGTRLGDIYRELWRSGVTVPAGTCPRIGIGGHVLGGGMGVLSRSRGALVDHLAALEFVDAEGRAYRVDENEHPDLFWACRGGGGGSYGIATSYTLRTSPVDEVTLFTLSWNWERFAPAVRAWQSWLASTDDRVNTFLSLFPRQQDLVAAFGVYDGPAAEFEELLRPLVAAVRPQDQVVEPMPFIQAVDTVELLQGEAAAADQVRAQGSSAIIGGPLDGAAIATLQQFLTDPPSHRSEVAVYGMGGAIARPARTDTAFVHRTGLMAFEYRTDWDRSEDDAKNLAWIKALRHAMAPHTTGAAYVNTIDLALENWLWAYYEENLPRLIEVKRRYDPGDVFHHPHSIPLTVTEADIARYGIPEAVAARLRAAGLTDRQ

SEQ ID NO: 116 - protein, 475 aa, synthetic construct
MATSARFAGTVLAPGDPGFEDASRTWNACFASRPREVMVCHSTAEVADAVREVRERGVSFRVRSGGHSMAGLSSVEDGVVIDLGGLDAISLSADRSLVTVGGGTRLGDIYRELWRSGVTVPAGTCPRIGIGGHVLGGGMGVLSRSRGALVDHLAALEFVDAEGRAYRVDENEHPDLFWACRGGGGGSYGIATSYTLRTSPVDEVTLFTLSWNWERFAPAVRAWQSWLASTDDRVNTFLSLFPRQQDLVAAFGVYDGPAAEFEELLRPLVAAVRPQDQVVEPMPFIQAVDTVELLQGEAAAADQVRAQGSSAIIGGPLDGAAIATLQQFLTDPPSHRSEVAVYGMGGAIARPARTDTAFVHRTGLMAFNYRTDWDRSEDDAKNLAWIKALRHAMAPHTTGAAYVNTIDLALENWLWAYYEENLPRLIEVKRRYDPGDVFHHPHSIPLTVTEADIARYGIPEAVAARLRAAGLTDRQ

SEQ ID NO: 117 - protein, 473 aa, synthetic construct
MSTAPVFGGRIVTRLDPSYEEARQVWNAVPDHRPLEIRYCRDAQDVAAALRDALARGLPFAARSGGHSAAALSVVPDGVVIDVSPLNSISLDQDASQVTFGSGATLGAIYRTLWESGVTVPAGTCPNIGVAGHVLGGGLGILSRSRGPLVDHLIGVTMVDARGELVTADEENHSDLLWACRGGGGGNYGIATSFTLRTQPIADVTLFNITWRFDDLGPAVKAWQSWLADADHRINPFMMLYPREQDMVATVGVMEGTPDAFEPLVKPLLDAVEPSEVVIEPMSFIQAVDTVEALQGEAAVAKSVRALGSSAIIDRPLDDRALETLRRHLIDPPSHRSEVAIYGMGGKIAAKGREETAFVHRTELMAFEYRTDWDEPADDEKNVAWVRSVRQEMAEHTTGGTYTNTVDLGVENWLWGYYEENLPRLMGVKHRYDPDGVFQHAHSVPLSLTEAEASRFGIPEQIRRTLRAAGRLT

SEQ ID NO: 118 - protein, 516 aa, synthetic construct; identical to SEQ ID NO: 1

SEQ ID NO: 119 - protein, 516 aa, synthetic construct; identical to SEQ ID NO: 3

SEQ ID NO: 120 - protein, 516 aa, synthetic construct; identical to SEQ ID NO: 4

SEQ ID NO: 121 - protein, 517 aa, synthetic construct; identical to SEQ ID NO: 27

SEQ ID NO: 122 - protein, 517 aa, synthetic construct
NPRENFLKCFSKHIPNNATNPKLVYTQHDQLYMSILNSTIQNLRFISDTTPKPLVIVTPSQNSHIQATILCSKKVGLQIRTRSGGHDAEGMSYISQVPFVVVDLRNMHSIKIDVHSQTAWVEAGATLGEVYYWINEKNEQLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFMTPYVSQNPRLAYLNYRDLDLGKTNHASPNNYTQARIWGEKYFGKNFNRLVKVKTKVDPNNFFRNEQSIPPLPPHHH

SEQ ID NO: 123 - protein, 520 aa, synthetic construct
NPRENFLKCFSKHIPNNATNPKLVYTQHDQLYMSILNSTIQNLRFISDTTPKPLVIVTPSQNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKQITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFMTPYVSQNPRLAYLNYRDLDLGKTNHASPNNYTQARIWGEKYFGKNFNRLVKVKTKVDPNNFFRNEQSIPPLPPHHHHHH

SEQ ID NO: 124 - protein, 517 aa, synthetic construct
NPRENFLKCFSKHIPNNATNPKLVYTQHDQLYMSILNSTIQNLRFISDTTPKPLVIVTPSQNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKQKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFMTPYVSQNPRLAYLNYRDLDLGKTNHASPNNYTQARIWGEKYFGKNFNRLVKVKTKVDPNNFFRNEQSIPPLPPHHH

SEQ ID NO: 125 - protein, 517 aa, synthetic construct
NPRENFLKCFSKHIPNNATNPKLVYTQHDQLYMSILNSTIQNLRFISDTTPKPLVIVTPSQNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMQKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFMTPYVSQNPRLAYLNYRDLDLGKTNHASPNNYTQARIWGEKYFGKNFNRLVKVKTKVDPNNFFRNEQSIPPLPPHHH

SEQ ID NO: 126 - protein, 517 aa, synthetic construct
NPRENFLKCFSKHIPNNATNPKLVYTQHDQLYMSILNSTIQNLRFISDTTPKPLVIVTPSQNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFMTPYVSQNPRLAYLNYRDLDLGKTNHASPNQYTQARIWGEKYFGKNFNRLVKVKTKVDPNNFFRNEQSIPPLPPHHH